(19) United States
(12) Patent Application Publication
(10) Pub. No.: US 2024/0018571 A1
(43) Pub. Date: Jan. 18, 2024

(54) METHODS, COMPOSITIONS, AND KITS FOR NUCLEIC ACID DETECTION

(71) Applicant: MESO SCALE TECHNOLOGIES, LLC., Rockville, MD (US)

(72) Inventors: John KENTEN, Boyds, MD (US); Jacob N. WOHLSTADTER, Palm Beach Gardens, FL (US)

(21) Appl. No.: 17/999,076

(22) PCT Filed: May 18, 2021
(86) PCT No.: PCT/US2021/032886

§ 371 Date: Nov. 17, 2022

Related U.S. Application Data
(60) Provisional application No. 63/175,930, filed on Apr. 16, 2021.
(60) Provisional application No. 63/039,890, filed on Jun. 16, 2020.
(60) Provisional application No. 63/032,031, filed on May 29, 2020.
(60) Provisional application No. 63/027,234, filed on May 19, 2020.

(51) Int. Cl. C12Q 1/6818

(52) U.S. Cl. CPC C12Q 1/6818

(57) ABSTRACT

The invention relates to methods, oligonucleotide reagents, compositions, and kits for nucleic acid detection.

### FIELD OF THE INVENTION

The invention relates to methods, compositions, and kits for nucleic acid detection.

### BACKGROUND

Highly sensitive and specific detection of nucleic acids has the potential to revolutionize diagnosis and monitoring of diseases, provide valuable epidemiological information, and serve as a generalizable scientific tool. Nucleic acid-based assay systems typically require high sensitivity, e.g., with the detection limit to be as low as tens of molecules per sample, in combination with single base-pair level of specificity. Current approaches, e.g., qPCR, are analytically sensitive but require a lengthy, complex, and expensive processing procedure.

### SUMMARY OF THE INVENTION

In embodiments, the invention provides a method for detecting a nucleic acid of interest in a sample, comprising: (a) contacting the sample with an oligonucleotide binding reagent, wherein the oligonucleotide binding reagent comprises: (i) a targeting agent complement; (ii) an amplification primer; (iii) a hybridization region comprising a complementary sequence to the nucleic acid of interest; and (iv) an amplification blocker; (b) forming a binding complex comprising the nucleic acid of interest and the oligonucleotide binding reagent; (c) contacting the binding complex with a site-specific nuclease that cleaves the oligonucleotide binding reagent to remove the amplification blocker therefrom, thereby generating a first cleaved oligonucleotide comprising the targeting agent complement and the amplification primer, wherein the first cleaved oligonucleotide is not bound to the nucleic acid of interest; (d) immobilizing the first cleaved oligonucleotide to a detection surface comprising a targeting agent, wherein the targeting agent is a binding partner of the targeting agent complement; (e) extending the first cleaved oligonucleotide on the detection surface to form an extended oligonucleotide; and (f) detecting the extended oligonucleotide, thereby detecting the nucleic acid of interest in the sample.

In embodiments, the invention provides a method for detecting a nucleic acid of interest in a sample, comprising: (a) contacting the sample with a site-specific nuclease comprising collateral cleavage activity and an oligonucleotide detection reagent, wherein the oligonucleotide detection reagent comprises: (i) a targeting agent complement; (ii) an amplification primer; and (iii) an amplification blocker, wherein the site-specific nuclease binds to the nucleic acid of interest and collaterally cleaves the oligonucleotide detection reagent to remove the amplification blocker therefrom, thereby generating a first cleaved oligonucleotide comprising the targeting agent complement and the amplification primer; (b) immobilizing the first cleaved oligonucleotide to a detection surface comprising a targeting agent, wherein the targeting agent is a binding partner of the targeting agent complement; (c) extending the first cleaved oligonucleotide to form an extended oligonucleotide; and (d) detecting the extended oligonucleotide, thereby detecting the nucleic acid of interest in the sample.

In embodiments, the invention provides a method for detecting a nucleic acid of interest in a sample, comprising: (a) contacting the sample with a site-specific nuclease comprising collateral cleavage activity and an oligonucleotide detection reagent, wherein the oligonucleotide detection reagent comprises: (i) a primary targeting agent complement; (ii) a secondary targeting agent complement; and (iii) a detectable label; wherein the site-specific nuclease binds to the nucleic acid of interest and collaterally cleaves the oligonucleotide detection reagent, thereby generating (i) a cleaved secondary targeting agent complement and (ii) a first cleaved oligonucleotide comprising the primary targeting agent complement and the detectable label; (b) binding the cleaved secondary targeting agent complement, uncleaved oligonucleotide detection reagent, or both, to a binding surface comprising a secondary targeting agent that is a binding partner of the secondary targeting agent complement; (c) immobilizing the first cleaved oligonucleotide to a detection surface comprising a primary targeting agent, wherein the primary targeting agent is a binding partner of the primary targeting agent complement; and (d) detecting the first cleaved oligonucleotide bound to the detection surface, wherein the secondary targeting agent complement and the uncleaved oligonucleotide detection reagent on the binding surface are substantially undetected, thereby detecting the nucleic acid of interest in the sample.

In embodiments, the invention provides a method for detecting a nucleic acid of interest in a sample, comprising: (a) contacting the sample with a site-specific nuclease comprising collateral cleavage activity, and an oligonucleotide detection reagent, wherein the oligonucleotide detection reagent comprises: (i) a targeting agent complement; (ii) a targeting agent blocker that is complementary to at least a portion of the targeting agent complement; (iii) a nuclease cleavage site; and (iv) a detectable label; wherein the targeting agent complement and the targeting agent blocker are hybridized, wherein the site-specific nuclease binds to the nucleic acid of interest and collaterally cleaves the oligonucleotide detection reagent at the nuclease cleavage sequence, thereby (i) destabilizing hybridization of the targeting agent complement and the targeting agent blocker and (ii) generating an unblocked oligonucleotide comprising the targeting agent complement and the detectable label; (b) immobilizing the unblocked oligonucleotide to a detection surface comprising a targeting agent, wherein the targeting agent is a binding partner of the targeting agent complement, wherein uncleaved oligonucleotide detection reagent does not substantially bind to the detection surface; and (c) detecting the unblocked oligonucleotide bound to the detection surface, thereby detecting the nucleic acid of interest in the sample.

In embodiments, the invention provides an oligonucleotide binding reagent comprising: (i) a targeting agent complement (TAC); (ii) an amplification primer; and (iii) an amplification blocker. In embodiments, the invention provides a composition comprising: the oligonucleotide binding reagent and one or both of: a site-specific nuclease and a nucleic acid of interest.

In embodiments, the invention provides an oligonucleotide detection reagent comprising: (i) a targeting agent complement (TAC); (ii) an amplification primer; and (iii) an amplification blocker. In embodiments, the invention provides a composition comprising: the oligonucleotide detection reagent, a site-specific nuclease, and a nucleic acid of interest.

In embodiments, the invention provides an oligonucleotide detection reagent comprising: (i) a primary targeting agent complement (primary TAC); (ii) a secondary targeting agent complement (secondary TAC); and (iii) a detectable label. In embodiments, the invention provides a composition comprising the oligonucleotide detection reagent, a site-specific nuclease, and a nucleic acid of interest.

In embodiments, the invention provides an oligonucleotide detection reagent comprising: (i) a targeting agent complement (TAC); (ii) a targeting agent blocker that is complementary to at least a portion of the TAC; (iii) a nuclease cleavage site; and (iv) a detectable label. In embodiments, the invention provides a composition comprising the oligonucleotide detection reagent, a site-specific nuclease, and a nucleic acid of interest.

### BRIEF DESCRIPTION OF THE DRAWINGS

The following drawings form part of the present specification and are included to further demonstrate exemplary embodiments of certain aspects of the present invention.

FIG. 1 illustrates an embodiment of a method described herein. An oligonucleotide binding reagent comprises, in 5′ to 3′ order, a targeting agent complement (TAC), an amplification primer, a target hybridization region, an amplification blocker, and a secondary targeting agent complement (TAC). The oligonucleotide binding reagent hybridizes with a nucleic acid of interest to form a binding complex. The binding complex is contacted with a Cas nickase, which nicks the oligonucleotide binding reagent to form: (i) a first cleaved oligonucleotide that comprises the TAC and the amplification primer, wherein the first cleaved oligonucleotide is activated for amplification and dissociates from the nucleic acid of interest and the Cas nickase; and (ii) a second cleaved oligonucleotide that comprises the amplification blocker and the secondary TAC. Following the cleavage, the Cas nickase binds to a further binding complex comprising a further copy of the oligonucleotide binding reagent and the nucleic acid of interest and cleaves the further copy of the oligonucleotide binding reagent to generate a further first cleaved oligonucleotide and a further second cleaved oligonucleotide. After one or more cycles of Cas nickase binding and cleavage, the reaction mixture comprising the one or more first and second cleaved oligonucleotides is incubated on a binding surface comprising a secondary targeting agent, which removes any uncleaved oligonucleotide binding reagent and second cleaved oligonucleotides. Following the incubation on the binding surface, the reaction mixture is incubated on a detection surface comprising a targeting agent to immobilize the one or more first cleaved oligonucleotides onto the detection surface. In embodiments, the immobilized first cleaved oligonucleotide(s) are subjected to extension and detection as described herein.

FIG. 2 illustrates an embodiment of a method described herein. An oligonucleotide detection reagent comprises, in 5′ to 3′ order, a targeting agent complement (TAC), an amplification primer, a nuclease cleavage site, and an amplification blocker. A Cas13 complex binds a nucleic acid of interest, thereby activating collateral cleavage activity of the Cas13. The Cas13 collaterally cleaves one or more copies of the oligonucleotide detection reagent, thereby generating one or more first cleaved oligonucleotides, each comprising the TAC and the amplification primer. The reaction mixture sample is incubated on a detection surface comprising a targeting agent to immobilize the one or more first cleaved oligonucleotides onto the detection surface. In embodiments, the immobilized first cleaved oligonucleotide(s) are subjected to extension and detection as described herein.

FIG. 3 illustrates an embodiment of a method described herein. An oligonucleotide detection reagent comprises, in 5′ to 3′ order, a secondary targeting agent complement (TAC), a nuclease cleavage site, a primary TAC, and a detectable label. A Cas13 complex binds a nucleic acid of interest, thereby activating collateral cleavage activity of the Cas13. The Cas13 collaterally cleaves one or more copies of the oligonucleotide detection reagent, thereby generating one or more of: (i) first cleaved oligonucleotides, each comprising the primary TAC and detectable label; and (ii) second cleaved oligonucleotides, each comprising the secondary TAC. The reaction mixture sample is incubated on a binding surface comprising a secondary targeting agent to bind the one or more second cleaved oligonucleotides, thereby separating the second cleaved oligonucleotide(s) from the first cleaved oligonucleotide(s). The resulting reaction mixture sample is then incubated on a detection surface comprising a primary targeting agent to immobilize the one or more first cleaved oligonucleotides onto the detection surface. In embodiments, the detectable label(s) of the immobilized first cleaved oligonucleotide(s) are detected as described herein.

FIG. 4A illustrates an embodiment of an oligonucleotide detection reagent described herein. An oligonucleotide detection reagent comprises, in 5′ to 3′ order, a targeting agent blocker, a nuclease cleavage site, a targeting agent complement, and a detectable label. The targeting agent blocker is hybridized to the targeting agent complement. In embodiments, the nuclease cleavage site comprises a hairpin loop structure. In embodiments, the nuclease cleavage site is capable of being cleaved by a site-specific nuclease, as described herein.

FIG. 4B illustrates an embodiment of an oligonucleotide detection reagent described herein. An oligonucleotide detection reagent comprises first and second strands, wherein a targeting agent complement is on the first strand, and a targeting agent blocker and a nuclease cleavage site are on the second strand. The targeting agent blocker comprises a first region and a second region, wherein the nuclease cleavage site is positioned between the first region and the second region of the targeting agent blocker. The first and second regions of the targeting agent blocker hybridize to first and second regions of the targeting agent complement. In embodiments, the nuclease cleavage site comprises a hairpin loop structure. In embodiments, the nuclease cleavage site is capable of being cleaved by a site-specific nuclease, as described herein.

### DETAILED DESCRIPTION OF THE INVENTION

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

As used herein, “complementary” in reference to an oligonucleotide means that the oligonucleotide or one or more regions thereof is capable of hydrogen bonding with a second oligonucleotide or one or more regions thereof. Complementary oligonucleotides and/or nucleic acids need not have complementarity at each nucleotide and may include one or more nucleotide mismatches, i.e., points at which hydrogen bonding does not occur. For example, complementary oligonucleotides can have at least 80%, at least 85%, at least 90%, at least 95%, at least 96%, at least 97%, at least 98%, or at least 99% of nucleotides hydrogen bond. By contrast, “fully complementary” or “100% complementary” in reference to oligonucleotides means that each nucleotide hydrogen bonds without any nucleotide mismatches.

The present invention provides highly sensitive and specific methods and kits for the detection of nucleic acids of interest. For example, the present invention provides an isothermal assay format that combines the specificity of the bacterial CRISPR/Cas system for targeted and non-specific cleavage of nucleic acids, with the sensitivity of the amplification and detection methods described herein. The present methods advantageously utilize the non-specific cleavage activity of Cas nucleases to further amplify the assay signal, thereby further increasing the sensitivity. Moreover, the present methods can be performed in a multiplexed format that can simultaneously detect multiple nucleic acids of interest, thereby reducing the sample volume requirement as well as time and resources otherwise required for performing multiple individual assays.

### Assay Embodiment I. Nickase Cleavage, Extension and Detection

In embodiments, the invention provides a method for detecting a nucleic acid of interest in a sample, comprising: (a) contacting the sample with an oligonucleotide binding reagent, wherein the oligonucleotide binding reagent comprises: (i) a targeting agent complement (TAC); (ii) an amplification primer; (iii) a hybridization region comprising a complementary sequence to the nucleic acid of interest; and (iv) an amplification blocker; (b) forming a binding complex comprising the nucleic acid of interest and the oligonucleotide binding reagent; (c) contacting the binding complex with a site-specific nuclease that cleaves the oligonucleotide binding reagent to remove the amplification blocker therefrom, thereby generating a first cleaved oligonucleotide comprising the TAC and the amplification primer, wherein the first cleaved oligonucleotide is not bound to the nucleic acid of interest; (d) immobilizing the first cleaved oligonucleotide to a detection surface comprising a targeting agent, wherein the targeting agent is a binding partner of the TAC; (e) extending the first cleaved oligonucleotide on the detection surface to form an extended oligonucleotide; and (0 detecting the extended oligonucleotide, thereby detecting the nucleic acid of interest in the sample.

In embodiments, the nucleic acid of interest is a double-stranded oligonucleotide. In embodiments, the nucleic acid of interest is a single-stranded oligonucleotide. In embodiments, the nucleic acid of interest is DNA, e.g., single-stranded DNA (ssDNA) or double-stranded DNA (dsDNA). In embodiments, the nucleic acid of interest is RNA, e.g., single-stranded RNA (ssRNA) or double-stranded RNA (dsRNA). Unless specified otherwise, it will be understood in the present disclosure that “DNA” refers to dsDNA, and “RNA” refers to ssRNA. Exemplary nucleic acids of interest and samples are provided herein.

In embodiments, the sample comprising the nucleic acid of interest is contacted with the oligonucleotide binding reagent. In embodiments, the oligonucleotide binding reagent binds to the nucleic acid of interest. In embodiments, the oligonucleotide binding reagent comprises a single-stranded oligonucleotide. In embodiments, the oligonucleotide binding reagent comprises a TAC, an amplification primer, a hybridization region, and an amplification blocker. In embodiments, the oligonucleotide binding reagent comprises, in 5′ to 3′ order, the TAC, the amplification primer, the hybridization region, and the amplification blocker.

In embodiments, the oligonucleotide binding reagent is about 20 to about 300, about 25 to about 280, about 30 to about 250, about 35 to about 220, about 40 to about 200, about 45 to about 180, about 50 to about 150, about 55 to about 120, about 60 to about 100, or about 65 to about 80 nucleotides in length.

In embodiments, the amplification primer comprises a primer for polymerase chain reaction (PCR), ligase chain reaction (LCR), strand displacement amplification (SDA), self-sustained synthetic reaction (3SR), or an isothermal amplification method. In embodiments, the amplification primer comprises a primer for an isothermal amplification method. In embodiments, the isothermal amplification method is helicase-dependent amplification. In embodiments, the isothermal amplification method is rolling circle amplification (RCA). Amplification methods are further described, e.g., in Carrino et al., J Microbiol Method 23(1):3-20 (1995); Fakruddin et al., J Pharm Bioallied Sci 5(4):245-252 (2013); and Nolte et al., “Chapter 1: Nucleic Acid Amplification Methods Overview” in Molecular Microbiology: Diagnostic Principles and Practice, 3^{rd} Ed. (2016), ASM Press. RCA is further described, e.g., in Barter et al., Nucleic Acids Res, 26:5073-5078 (1998); Lizardi et al., Nature Genetics 19:226 (1998); Schweitzer et al., Proc Natl Acad Sci USA 97:10113-10119 (2000); Faruqi et al., BMC Genomics 2:4 (2000); Nallur et al., Nucleic Acids Res 29:e118 (2001); Dean et al. Genome Res 11:1095-1099 (2001); Schweitzer et al., Nature Biotechnol 20:359-365 (2002); and U.S. Pat. Nos. 6,054,274; 6,291,187; 6,323,009; 6,344,329; and 6,368,801. In embodiments, the amplification primer is about 1 to about 50, about 5 to about 45, about 10 to about 40, about 12 to about 35, about 15 to about 30, about 18 to about 40, about 20 to about 35, about 25 to about 30, or about 30 to about 35 nucleotides in length. In embodiments, the amplification primer is about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In embodiments, the amplification primer comprises the sequence GACAGAACTAGACAC (SEQ ID NO:64).

In embodiments, the amplification blocker blocks amplification of the amplification primer. In embodiments, the amplification blocker comprises an oligonucleotide that blocks amplification of the amplification primer by preventing polymerase binding, inhibiting polymerase activity, and/or promoting polymer dissociation from the amplification primer. In embodiments, the amplification blocker comprises a nucleotide modification. Non-limiting examples of nucleotide modifications that block amplification include 3′-spacer C3, 3′-phosphate, 3′-dideoxy cytidine (3′-ddC), and 3′-inverted end. In embodiments, the amplification blocker comprises a peptide nucleic acid (PNA) and/or a locked nucleic acid (LNA). In embodiments, the amplification blocker comprises a 2′-O-methyl uridine, a 3′-inverted dT, a digoxigenin, a biotin, or a combination thereof. In embodiments, the amplification blocker comprises a secondary structure, e.g., a stem loop or a pseudoknot.

In embodiments, the hybridization region of the oligonucleotide binding reagent comprises a complementary sequence to the nucleic acid of interest. In embodiments, the hybridization region binds the nucleic acid of interest, thereby forming a binding complex comprising the oligonucleotide binding reagent and the nucleic acid of interest. In embodiments where the nucleic acid of interest is single-stranded, the binding complex comprises a double-stranded duplex formed by the nucleic acid of interest and the oligonucleotide binding reagent. In embodiments where the nucleic acid of interest is double-stranded and one strand of the nucleic acid of interest is complementary to the hybridization region, the method further comprises separating the strands of the double-stranded nucleic acid of interest and forming a double-stranded duplex between one strand of the nucleic acid of interest and the oligonucleotide binding reagent. In embodiments, the nucleic acid of interest is a polynucleotide in a sample, wherein the entire polynucleotide hybridizes to the oligonucleotide binding reagent in the binding complex. In embodiments, the nucleic acid of interest is a portion or region of another compound, e.g., a longer polynucleotide, wherein a portion of the longer polynucleotide does not hybridize to the oligonucleotide binding reagent in the binding complex.

In embodiments, the hybridization region is about 10 to about 50, about 11 to about 45, about 12 to about 40, about 13 to about 35, about 14 to about 30, about 15 to about 25, about 16 to about 24, about 17 to about 23, or about 18 to about 22 nucleotides in length. In embodiments, the hybridization region is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In embodiments, the oligonucleotide binding reagent further comprises a nuclease binding site. In embodiments, the nuclease binding site is positioned between the hybridization region and the amplification blocker. In embodiments, the nuclease binding site comprises at least a portion of the hybridization region, at least a portion of the amplification blocker, or both.

In embodiments, the binding complex is contacted with a site-specific nuclease. In embodiments, the site-specific nuclease is a nickase that is capable of cleaving one strand of a double-stranded duplex, e.g., the double-stranded duplex formed by the nucleic acid of interest and the oligonucleotide binding reagent. In embodiments, the site-specific nuclease is an RNA-guide nickase. In embodiments, the RNA-guided nickase is a Cas9 nickase or a Cas12a (also known as Cpf1) nickase. Cas9 and Cas12a nickases are described, e.g., in Mali et al., Nat Biotechnol 31:833-838 (2013); Ran et al., Cell 155(2):479-480 (2013); Trevino et al., Methods Enzymol 546:161-174 (2014); Fu et al., Nat Microbiol 4:888-897 (2019); and Standage-Beier et al., ACS Synth Biol 4:1217-1225 (2015). Exemplary Cas9 and Cas12a nucleases are provided in Tables 1 and 2, respectively.

TABLE 1
Exemplary Cas9 Proteins

| Bacterial Species | Protein Name |
|---|---|
| Streptococcus pyogenes | SpCas9 |
| Staphylococcus aureus | SaCas9 |
| Streptococcus thermophilus | StCas9 |
| Neisseria meningitidis | NmCas9 |
| Francisella novicida | FnCas9 |
| Campylobacter jejuni | CjCas9 |
| Streptococcus canis | ScCas9 |

TABLE 2
Exemplary Cas12 Proteins

| Bacterial Species | Cas12 subclass | Protein Name |
|---|---|---|
| Lachnospiraceae bacterium ND2006 | Cas12a | LbaCas12a |
| Acidaminococcus sp. | Cas12a | AsCas12a |
| Francisella novicida | Cas12a | FnCas12a |
| Alicyclobacillus acidoterrestris | Cas12b | AaCas12b |

In embodiments, the site-specific nuclease, e.g., Cas9 nickase or Cas12a nickase, forms a complex with a guide polynucleotide, e.g., a guide RNA. In general, “guide RNA” refers to a nucleic acid comprising a tracrRNA, which binds the Cas enzyme (e.g., any of the Cas proteins described herein, including Cas9 nickase, Cas12a nickase, and Cas13) and a crRNA, which is complementary to a target sequence (e.g., nucleic acid of interest). In embodiments, the guide RNA is a single guide RNA (sgRNA) comprising both the tracrRNA and the crRNA. In embodiments, the guide RNA comprises the tracrRNA and the crRNA on separate polynucleotides. Methods of designing and making guide RNA to form a complex with a Cas9 nickase or Cas12a nickase are known in the field and described, e.g., in Doench et al., Nat Rev Genet 19:67-80 (2017); Hanna et al., Nat Biotechnol 38:813-823 (2020); and Gootenberg et al., Science 360:439-444 (2018).

In embodiments, the guide polynucleotide comprises a complementary sequence to the nuclease binding site of the oligonucleotide binding reagent. In embodiments, the guide polynucleotide comprises a complementary sequence to the nucleic acid of interest. In embodiments, the site-specific nuclease, e.g., Cas9 nickase or Cas12a nickase, binds to the binding complex comprising the nucleic acid of interest and the oligonucleotide binding reagent via complementarity between the guide polynucleotide and the nuclease binding site, or via complementarity between the guide polynucleotide and the nucleic acid of interest. In embodiments, the site-specific nuclease, e.g., Cas9 nickase or Cas12a nickase, generates a single-stranded break in the oligonucleotide binding reagent of the binding complex. In embodiments, the site-specific nuclease does not generate a break in the nucleic acid of interest of the binding complex. In embodiments, the single-stranded break in the oligonucleotide binding reagent removes the amplification blocker from the oligonucleotide binding reagent to form a first cleaved oligonucleotide comprising the TAC and the amplification primer. In embodiments, the single-stranded break destabilizes the double-stranded duplex comprising the oligonucleotide binding reagent and the nucleic acid of interest. In embodiments, the first cleaved oligonucleotide dissociates from the nucleic acid of interest.

In embodiments, the TAC is a binding partner of a targeting agent on a detection surface. In embodiments, the TAC and the targeting agent comprise a binding pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the targeting agent and TAC are cross-reactive moieties, e.g., thiol and maleimide or iodoacetamide; aldehyde and hydrazide; or azide and alkyne or cycloalkyne. In embodiments, the TAC and the targeting agent comprise complementary oligonucleotides. In embodiments, the TAC and the targeting agent are at least 90%, at least 95%, at least 98%, at least 99%, or 100% complementary. In embodiments, the TAC is an oligonucleotide of about 5 to about 100, about 6 to about 90, about 7 to about 80, about 8 to about 70, about 9 to about 60 nucleotides, about 10 to about 50, about 15 to about 45, about 20 to about 40, about 20 to about 30, about 20 to about 35, or about 30 to about 35 nucleotides in length. In embodiments, the TAC comprises any of SEQ ID NOs:68-71. In embodiments, the method comprises immobilizing the first cleaved oligonucleotide to the detection surface via binding of the TAC to the targeting agent. Detection surfaces are further described herein.

In embodiments, the oligonucleotide binding reagent further comprises a secondary targeting agent complement (secondary TAC). In embodiments, the secondary TAC is a binding partner of a secondary targeting agent on a binding surface. In embodiments, the secondary TAC and the secondary targeting agent comprise a binding pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the secondary targeting agent and secondary TAC are cross-reactive moieties, e.g., thiol and maleimide or iodoacetamide; aldehyde and hydrazide; or azide and alkyne or cycloalkyne. In embodiments, the TAC and the targeting agent are substantially unreactive with the secondary TAC and the secondary targeting agent. As used herein, the term “substantially unreactive” means that less than 10%, less than 5%, less than 2%, or less than 1% of either of the TAC or targeting agent reacts with either of the secondary TAC or secondary targeting agent. In embodiments, the TAC and the targeting agent comprise complementary oligonucleotides, and the secondary TAC and the secondary targeting agent comprise a non-oligonucleotide binding pair. In embodiments, the TAC and the targeting agent comprise complementary oligonucleotides, and the secondary TAC and the secondary targeting agent comprise complementary oligonucleotides that are substantially non-hybridizable to the TAC or targeting agent. As used herein, “substantially non-hybridizable” means that under standard nucleic acid hybridization conditions, less than 10%, less than 5%, less than 2%, or less than 1% of either of the TAC or targeting agent hybridizes with either of the secondary TAC or secondary targeting agent. Standard nucleic acid hybridization conditions are described, e.g., in Herzer and Englert, “Chapter 14. Nucleic Acid Hybridization,” in Molecular Biology Problem Solver: A Laboratory Guide, (2001) ed. Alan S. Gersterin; Wiley-Liss, Inc. In embodiments, the amplification blocker is a binding partner of the secondary targeting agent on the binding surface. In embodiments, the secondary TAC and/or the amplification blocker comprises biotin, and the secondary targeting agent comprises avidin or streptavidin. In embodiments, the secondary TAC and/or the amplification blocker comprises digoxigenin, and the secondary targeting agent comprises an anti-digoxigenin antibody. In embodiments, the secondary TAC is an oligonucleotide of about 5 to about 100, about 6 to about 90, about 7 to about 80, about 8 to about 70, about 9 to about 60 nucleotides, about 10 to about 50, about 15 to about 45, about 20 to about 40, about 20 to about 30, or about 30 to about 35 nucleotides in length.

In embodiments, the TAC and the secondary TAC are on separate ends of the oligonucleotide binding reagent. In embodiments, the TAC is at a 5′ end of the oligonucleotide binding reagent, and the secondary TAC is at a 3′ end of the oligonucleotide binding reagent. In embodiments, the TAC is at a 3′ end of the oligonucleotide binding reagent, and the secondary TAC is at a 5′ end of the oligonucleotide binding reagent. In embodiments, the oligonucleotide binding reagent comprises, in 5′ to 3′ order: the TAC, the amplification primer, the hybridization region, the amplification blocker, and the secondary TAC. In embodiments, the secondary TAC is positioned adjacent to the amplification blocker on the oligonucleotide binding reagent, such that cleavage of the oligonucleotide binding reagent by the site-specific nuclease forms (i) the first cleaved oligonucleotide described herein and (ii) a second cleaved oligonucleotide comprising the amplification blocker and the secondary TAC.

In embodiments, a reaction mixture comprising the first cleaved oligonucleotide, second cleaved oligonucleotide, and uncleaved oligonucleotide binding reagent is formed following contacting the binding complex with the site-specific nuclease. In embodiments, the presence of uncleaved oligonucleotide binding reagent and/or second cleaved oligonucleotide interferes with one or more of the downstream steps of the method, e.g., immobilization, extension, and/or detection of the first cleaved oligonucleotide. In embodiments, the method further comprises, prior to the extending of the first cleaved oligonucleotide, removing the second cleaved oligonucleotide, uncleaved oligonucleotide binding reagent, or both. In embodiments, the removing comprises contacting the reaction mixture (comprising the first cleaved oligonucleotide, second cleaved oligonucleotide, and uncleaved oligonucleotide binding reagent) with the binding surface comprising the secondary targeting agent described herein, wherein the second cleaved oligonucleotide and the uncleaved oligonucleotide binding reagent bind to the binding surface. In embodiments, the removing further comprises separating the binding surface comprising the second cleaved oligonucleotide and the uncleaved oligonucleotide binding reagent from the reaction mixture. In embodiments, the first cleaved oligonucleotide does not comprise the secondary TAC and therefore does not bind to the binding surface. In embodiments, the binding of the second cleaved oligonucleotide and the uncleaved oligonucleotide binding reagent to the binding surface reduces or eliminates interference with the immobilization, extension, and/or detection of the first cleaved oligonucleotide. In embodiments, the method has increased specificity as compared to a method that does not remove the second cleaved oligonucleotide and/or the uncleaved oligonucleotide binding reagent from the reaction mixture.

In embodiments, the method comprises, following removal of the second cleaved oligonucleotide and/or the oligonucleotide binding reagent, immobilizing the first cleaved oligonucleotide to the detection surface via binding of the TAC to the targeting agent. Binding of the TAC to the targeting agent is further described herein.

In embodiments, the method comprises extending the immobilized first cleaved oligonucleotide on the detection surface to form an extended oligonucleotide. In embodiments, the extending comprises binding the amplification primer of the first cleaved oligonucleotide to a template oligonucleotide, and extending the amplification primer to form an extended oligonucleotide. In embodiments, the extending comprises polymerase chain reaction (PCR), ligase chain reaction (LCR), strand displacement amplification (SDA), self-sustained synthetic reaction (3SR), or an isothermal amplification method. In embodiments, the amplification primer comprises a primer for an isothermal amplification method. In embodiments, the isothermal amplification method is helicase-dependent amplification. In embodiments, the isothermal amplification method is rolling circle amplification (RCA). In embodiments, the template oligonucleotide for RCA comprises the sequence

(SEQ ID NO: 65)
GTTCTGTCATATTTCAGTGAATGCGAGTCCGTCTAAGAGAGTAGTACAG
CAAGAGTGTCTA.

In embodiments, the extended oligonucleotide comprises an anchoring region. In embodiments, the detection surface further comprises an anchoring reagent immobilized thereon. In embodiments, the anchoring region of the extended oligonucleotide binds to the anchoring reagent. In embodiments, the anchoring reagent comprises an oligonucleotide, aptamer, aptamer ligand, antibody, antigen, ligand, receptor, hapten, epitope, or mimotope. In embodiments, the anchoring reagent comprises a single-stranded oligonucleotide. In embodiments, the anchoring reagent comprises a double-stranded oligonucleotide. In embodiments, the anchoring reagent and the anchoring region comprise complementary oligonucleotides. In embodiments, the anchoring reagent comprises the sequence

(SEQ ID NO: 66)
AAGAGAGTAGTACAGCAGCCGTCAA.

In embodiments, binding the anchoring region to the anchoring reagent comprises forming a triple helix between the anchoring reagent and the extended oligonucleotide. In embodiments, binding the extended oligonucleotide to the anchoring reagent comprises: denaturing the anchoring region to expose a single-stranded region prior to the binding; exposing the anchoring region to helicase activity prior to the binding; and/or exposing the anchoring region to nuclease treatment prior to the binding, wherein the anchoring region comprises one or more hapten-modified bases and the anchoring reagent comprises one or more antibodies specific for the hapten; and/or the anchoring region comprises one or more ligand-modified bases and the anchoring reagent comprises one or more receptors specific for the ligand.

In embodiments, the method comprises detecting the extended oligonucleotide. In embodiments, the detecting comprises measuring the amount of extended oligonucleotide bound to the detection surface. In embodiments, the nucleic acid of interest is detected and/or quantified by measuring the amount of extended oligonucleotide bound to the detection surface. In embodiments, the detecting comprises: contacting the extended oligonucleotide with a labeled probe comprising a detectable label, wherein the labeled probe binds to the extended oligonucleotide; and measuring the amount of labeled probe bound to the extended oligonucleotide. In embodiments, the labeled probe and the extended oligonucleotide comprise complementary oligonucleotides. In embodiments, the labeled probe comprises the sequence

(SEQ ID NO: 67)
CAGTGAATGCGAGTCCGTCTAAG.

In embodiments, the extended oligonucleotide comprises a modified base, and measuring the amount of extended oligonucleotide comprises contacting the extended oligonucleotide with a detectable moiety that binds to the modified base. In embodiments, the modified base comprises an aptamer, aptamer ligand, antibody, antigen, ligand, receptor, hapten, epitope, or mimotope, and the detectable moiety comprises a binding partner of the modified base and a detectable label. In embodiments, the modified base comprises streptavidin or avidin, and the detectable moiety comprises (i) biotin and (ii) a detectable label. In embodiments, the modified base comprises biotin, and the detectable moiety comprises (i) streptavidin or avidin and (ii) a detectable label. Methods of detecting extended oligonucleotides are further described, e.g., in WO 2014/165061; WO 2014/160192; and WO 2015/175856.

In embodiments, the detectable label is detectable by light scattering, optical absorbance, fluorescence, chemiluminescence, electrochemiluminescence (ECL), bioluminescence, phosphorescence, radioactivity, magnetic field, or combinations thereof. In embodiments, the detectable label comprises phycoerythrin (PE). In embodiments, the detectable label comprises a β-galactosidase (β-gal) enzyme that can be detected by fluorescence detection when the β-gal enzyme cleaves a substrate such as resorufin-β-D-galactopyranoside to yield a fluorescent signal. In embodiments, the detectable label comprises an ECL label. In embodiments, the ECL label comprises an electrochemiluminescent organometallic complex. In embodiments, the electrochemiluminescent organometallic complex comprises ruthenium, osmium, iridium, rhenium, and/or a lanthanide metal. In embodiments, the ECL label comprises ruthenium. In embodiments, the electrochemiluminescent organometallic complex comprises a substituted or unsubstituted bipyridine or a substituted or unsubstituted phenanthroline. In embodiments, the ECL label comprises a substituted bipyridine. In embodiments, the ECL label comprises ruthenium (II) tris-bipyridine-(4-methylsulfone) or ruthenium (II) tris(bipyridine). In embodiments, the ECL label comprises ruthenium (II) tris-bipyridine-(4-methylsulfone). In embodiments, the ECL label comprises ruthenium (II) tris-bipyridine. Exemplary ECL labels are described, e.g., in U.S. Pat. Nos. 5,714,089, 6,316,607, 6,808,939, 9,499,573, 6,468,741, 6,479,233, and 6,136,268. ECL assays and instrumentation for conducting ECL assays are further described, e.g., in U.S. Pat. Nos. 5,093,268; 5,147,806; 5,240,863; 5,308,754; 5,324,457; 5,589,136; 5,591,581; 5,597,910; 5,641,623; 5,643,713; 5,679,519; 5,705,402; 5,731,147; 5,776,672; 5,786,141; 5,846,485; 5,866,434; 6,066,448; 6,136,268; 6,207,369; and 6,214,552; and PCT Publication Nos. WO 97/36931; WO 98/12539; WO 98/57154; WO 99/14599; WO 99/32662; WO 99/58962; WO 99/63347; and WO 00/03233.

In embodiments, the first cleaved oligonucleotide is not bound to the nucleic acid of interest (e.g., due to the single-stranded break in the oligonucleotide binding reagent destabilizing the double-stranded duplex formed by the oligonucleotide binding reagent and the nucleic acid of interest, as described herein), thereby allowing an additional copy of the oligonucleotide binding reagent to bind to the nucleic acid of interest. In embodiments, the method further comprises repeating one or more of the steps described herein, e.g.: contacting the sample comprising the nucleic acid of interest with an additional copy of the oligonucleotide binding reagent; forming a binding complex comprising the nucleic acid of interest and the additional copy of the oligonucleotide binding reagent; and contacting the binding complex with a site-specific nuclease to generate an additional copy of the first cleaved oligonucleotide; thereby generating a plurality of first cleaved oligonucleotides. In embodiments, the method comprises generating a plurality of first cleaved oligonucleotides from a single copy of the nucleic acid of interest. In embodiments, forming a plurality of first cleaved oligonucleotides amplifies the assay signal. In embodiments, the method has increased sensitivity for detecting the nucleic acid of interest as compared to a method that does not amplify the assay signal as described herein. In embodiments, the method is capable of detecting a lower amount of nucleic acid of interest in a sample as compared with a method that does not form the plurality of first cleaved oligonucleotides as described herein.

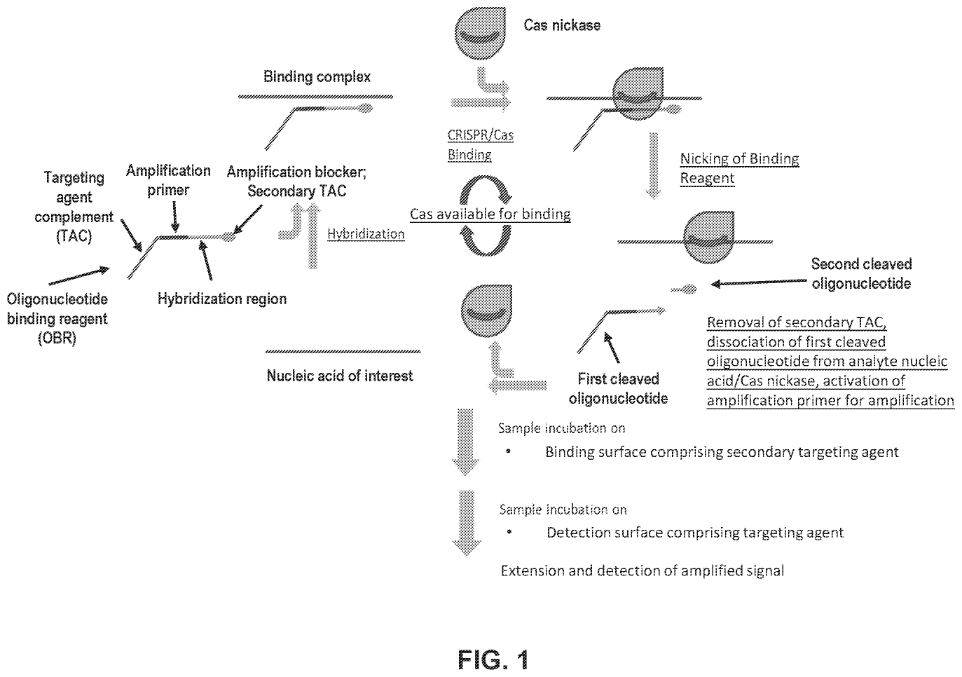

An embodiment of the method is illustrated in FIG. 1. In FIG. 1, an oligonucleotide binding reagent comprises, in 5′ to 3′ order, a TAC, an amplification primer, a target hybridization region, an amplification blocker, and a secondary TAC. The oligonucleotide binding reagent hybridizes with a nucleic acid of interest to form a binding complex. The binding complex is contacted with a Cas nickase, which nicks the oligonucleotide binding reagent to form: (i) a first cleaved oligonucleotide that comprises the TAC and the amplification primer, wherein the first cleaved oligonucleotide is activated for amplification and dissociates from the nucleic acid of interest and the Cas nickase; and (ii) a second cleaved oligonucleotide that comprises the amplification blocker and the secondary TAC. Following the cleavage, the Cas nickase binds to a further binding complex comprising a further copy of the oligonucleotide binding reagent and the nucleic acid of interest and cleaves the further copy of the oligonucleotide binding reagent to generate a further first cleaved oligonucleotide and a further second cleaved oligonucleotide. After one or more cycles of Cas nickase binding and cleavage, the reaction mixture comprising the one or more first and second cleaved oligonucleotides is incubated on a binding surface comprising a secondary targeting agent, which removes any uncleaved oligonucleotide binding reagent and second cleaved oligonucleotides. Following the incubation on the binding surface, the reaction mixture is incubated on a detection surface comprising a targeting agent to immobilize the one or more first cleaved oligonucleotides onto the detection surface. In embodiments, the immobilized first cleaved oligonucleotide(s) are subjected to extension and detection as described herein. Thus, as depicted in FIG. 1, the nucleic acid of interest is detected via detection of the immobilized first cleaved oligonucleotide(s).

An exemplary protocol for performing the method comprises:

1. Preparing the sample comprising the nucleic acid of interest. In embodiments, the preparing comprises extracting a nucleic acid (e.g., genomic DNA or RNA) from an organism of interest (e.g., a virus) that contains the nucleic acid of interest. In embodiments, the preparing further comprises producing cDNA from a genomic RNA, e.g., using reverse transcriptase.

2A. Incubating a sample reaction mixture, comprising the Cas enzyme (e.g., about 10 to about 100 nM, or about 20 to about 80 nM, or about 30 to about 60 nM, about 40 to 50 nM, or about 45 nM of purified Cas9 nickase or Cas12a nickase), guide RNA targeting the nucleic acid of interest (e.g., about 5 to about 50 nM, about 10 to about 40 nM, about 20 to about 30 nM, about 22 to about 25 nM, or about 22.5 nM), the oligonucleotide binding reagent (e.g., about 0.05 to about 100 nM, about 0.1 to about 80 nM, about 0.2 nM to about 60 nM, about 0.3 nM to about 50 nM, about 0.4 nM to about 40 nM, about 0.1 to about 20 nM, or about 0.1 to about 10 nM), and the sample that comprises the nucleic acid of interest. In embodiments, the TAC of the oligonucleotide binding reagent is biotin. In embodiments, the sample reaction mixture is in an assay buffer of pH about 6 to about 8, about 6.5 to about 7.5, or about 6.7 to about 7. In embodiments, the sample reaction mixture comprises a reaction volume of about 10 μL to about 1 mL, about 20 μL to about 700 μL, about 50 μL to about 500 μL, about 70 μL to about 200 μL, about 90 to about 150 μL, or about 100 μL. In embodiments, the sample reaction mixture is incubated for about 10 minutes to about 6 hours, about 30 minutes to about 4 hours, or about 1 hour to about 3 hours. In embodiments, the sample reaction mixture is incubated at about 20° C. to about 50° C., about 25° C. to about 45° C., about 30° C. to about 40° C., or about 37° C. In embodiments, the sample reaction mixture is incubated for about 1 hour to about 3 hours at about 37° C.

2B. Preparing an assay plate. In embodiments, the preparing comprises coating an assay plate with a targeting agent and an anchoring reagent. In embodiments, the targeting agent is streptavidin. In embodiments, the assay plate is a 96-well plate. In embodiments, the assay plate is coated with about 100 to about 500 ng of streptavidin, about 150 to about 400 ng of streptavidin, about 200 to about 350 ng of streptavidin, about 250 to about 300 ng of streptavidin, or about 275 ng of streptavidin. In embodiments, the assay plate is coated with about 100 to about 900 nM anchoring reagent, about 200 to about 700 nM anchoring reagent, about 300 to about 500 nM anchoring reagent, or about 400 nM anchoring reagent. In embodiments, the assay plate is washed, e.g., with PBS, following the coating. In embodiments, the assay plate is blocked with a blocking solution following the coating and washing. In embodiments, the blocking solution reduces and/or eliminates non-specific binding to the streptavidin and/or anchoring reagent on the assay plate.

In embodiments, steps 1 and 2 are performed simultaneously or substantially simultaneously. In embodiments, the producing of cDNA from a genomic RNA of step 1 and the incubating of step 2 are performed in the same reaction mixture.

3A. Incubating the sample reaction on the assay plate. In embodiments, about 10 to about 100 μL, about 20 to about 80 μL, about 30 to about 70 μL, about 40 to about 60 μL, or about 50 μL of the sample reaction is added to a well of the assay plate. In embodiments, the sample reaction is incubated for about 10 minutes to about 4 hours, about 30 minutes to about 2 hours, or about 1 hour. In embodiments, the sample reaction is incubated at about 15° C. to about 40° C., about 20° C. to about 37° C., about 25° C. to about 30° C., or about 27° C. In embodiments, the sample reaction is incubated for about 1 hour at about 27° C. In embodiments, the assay plate is washed, e.g., with PBS, following the incubating.

3B. Removing second cleaved oligonucleotide and/or uncleaved oligonucleotide binding reagent. In embodiments, the removing comprises contacting the sample reaction with magnetic beads comprising a secondary targeting agent. In embodiments, the secondary targeting agent is a binding partner of a secondary TAC and/or an amplification blocker on the oligonucleotide binding reagent. In embodiments, the magnetic beads are incubated with the sample reaction for about 10 minutes to about 4 hours, about 30 minutes to about 2 hours, or about 1 hour. In embodiments, the magnetic beads are incubated with the sample reaction at about 20° C. to about 50° C., about 25° C. to about 45° C., about 30° C. to about 40° C., or about 37° C. In embodiments, the magnetic beads are incubated with the sample reaction for about 1 hour at about 37° C. In embodiments, following the incubation, the beads are removed or separated (e.g., concentrated on a side of the sample reaction container such that the beads are no longer in contact with the sample reaction) from the sample reaction by contacting the sample reaction container with a magnet.

4. Performing an RCA reaction. RCA reactions are described, e.g., in U.S. Pat. No. 10,114,015. In embodiments, the RCA reaction comprises adding a ligation mix comprising ligase (e.g., T4 DNA ligase), ATP, template oligonucleotide, and ligation buffer to the sample reaction in the assay plate well. In embodiments, the sample reaction is incubated with the ligation mix for about 10 minutes to about 2 hours, about 20 minutes to about 1 hour, or about 30 minutes. In embodiments, the sample reaction is incubated with the ligation mix at about 15° C. to about 40° C., about 20° C. to about 37° C., about 25° C. to about 30° C., or about 22° C. to about 28° C. embodiments, the sample reaction is incubated for about 30 minutes at room temperature, e.g., about 22° C. to about 28° C. In embodiments, following incubation of the sample reaction and the ligation mix, a polymerase mix, comprising dNTPs (about 100 to about 500 μM, about 200 to about 400 μM, or about 250 μM of each of dATP, dGTP, dCTP), DNA polymerase (e.g., Phi29 DNA polymerase), and a labeled probe (e.g., about 1 to about 10 nM, about 2 to about 9 nM, about 4 to about 8 nM, about 6 to about 7 nM, about 5 nM, about 6 nM, or about 7 nM) as described herein, is added to the assay plate well to perform the RCA reaction. RCA reaction conditions are known in the art. In embodiments, the assay plate is washed, e.g., with PBS buffer, following the RCA reaction.

5. Reading the plate. In embodiments, about 50 to about 500 μL, about 100 to about 300 μL, or about 150 μL of a read buffer is added to the assay plate well. In embodiments, the assay plate is read on a plate reader immediately or substantially immediately following addition of the read buffer.

Virus Detection

In embodiments, the methods provided herein are used to detect a virus in a sample. In embodiments, the method detects a viral nucleic acid. In embodiments, the viral nucleic acid is viral DNA or viral RNA. In embodiments, the method is used to diagnose a viral infection in a subject. In embodiments, the virus is a respiratory virus, e.g., influenza A (FluA), influenza B (FluB), respiratory syncytial virus (RSV), a coronavirus, or a combination thereof. In embodiments, the virus is a coronavirus. In embodiments, the coronavirus is SARS-CoV-2.

In embodiments, the invention provides a method for detecting a coronavirus in a biological sample, comprising: a) contacting the biological sample with a binding reagent that specifically binds a nucleic acid of the coronavirus; b) forming a binding complex comprising the binding reagent and the coronavirus nucleic acid; and c) detecting the binding complex, thereby detecting the coronavirus in the biological sample. In embodiments, the binding reagent comprises an oligonucleotide comprising a sequence complementary to the coronavirus nucleic acid sequence. In embodiments, the coronavirus nucleic acid is RNA. In embodiments, the binding reagent comprises a single stranded oligonucleotide. In embodiments, the detecting comprises directly detecting the binding complex. In embodiments, the detecting comprises detecting one or more components of the binding complex, e.g., the binding reagent. In embodiments, the binding reagent is an oligonucleotide binding reagent described herein.

In embodiments, the binding reagent comprises one or more of: a TAC, an amplification primer, a target hybridization region, an amplification blocker, and a secondary TAC. In embodiments, the binding reagent is an oligonucleotide comprising, in 5′ to 3′ order: a TAC, an amplification primer, a target hybridization region, an amplification blocker, and a secondary TAC. TACs, amplification primers, amplification blockers, and secondary TACs of oligonucleotide binding reagents are described herein.

In embodiments, the target hybridization region comprises a complementary sequence to the nucleic acid of interest, as described herein. In embodiments, the target hybridization region comprises an oligonucleotide that is complementary to the coronavirus nucleic acid. In embodiments, the coronavirus nucleic acid is SARS-CoV-2 RNA.

In embodiments, the target hybridization region and/or the amplification blocker comprises a target nucleic acid for an RNA-guided nickase. RNA-guided nickases, including Cas9 nickase and Cas12a nickase, are further described herein.

In embodiments, the RNA-guided nickase forms a complex with a guide RNA that hybridizes to a target coronavirus nucleic acid (i.e., the nickase is “guided” to the target coronavirus nucleic acid via complementarity between the guide RNA and the target nucleic acid). In embodiments, the target nucleic acid is double-stranded. In embodiments, the target nucleic acid comprises the hybridized binding reagent and coronavirus nucleic acid. In embodiments, the binding reagent and the coronavirus nucleic acid each forms one “strand” of a double-stranded target nucleic acid. In embodiments, the method comprises contacting the binding complex comprising the binding reagent and the coronavirus nucleic acid with the RNA-guided nickase. In embodiments, the nickase generates a single-stranded break in the binding reagent. In embodiments, the single-stranded break removes the amplification blocker from the binding reagent to form a first cleaved oligonucleotide (also referred to herein as a “cleaved binding reagent”). In embodiments, the first cleaved oligonucleotide comprises the targeting agent complement and the amplification primer. In embodiments, the single-stranded break forms a second cleaved oligonucleotide (also referred to herein as a “cleaved amplification blocker-secondary targeting agent”). In embodiments, the second cleaved oligonucleotide comprises the amplification blocker and the secondary TAC. First cleaved oligonucleotides and second cleaved oligonucleotides are further described herein. In embodiments, the coronavirus nucleic acid is SARS-CoV-2 RNA.

In embodiments, the first cleaved oligonucleotide is not bound to the coronavirus nucleic acid, thereby allowing an additional copy of the binding reagent to bind to the coronavirus nucleic acid. In embodiments, the method further comprises repeating one or more steps to form a plurality of first cleaved oligonucleotides. In embodiments, the method comprises detecting the first cleaved oligonucleotides. In embodiments, the method comprises generating a plurality of first cleaved oligonucleotides from a single copy of the coronavirus nucleic acid. In embodiments, forming the plurality of first cleaved oligonucleotides amplifies the assay signal. In embodiments, the method has increased sensitivity of coronavirus detection as compared to a method that does not amplify the assay signal as described herein. In embodiments, the method is capable of detecting a lower amount of coronavirus nucleic acid in a biological sample as compared with a method that does not form the plurality of first cleaved oligonucleotides, as described herein. In embodiments, the coronavirus nucleic acid is SARS-CoV-2 RNA.

In embodiments, a reaction mixture containing the plurality of first cleaved oligonucleotides, uncleaved binding reagent, and second cleaved oligonucleotides is formed. In embodiments, the method comprises removing the second cleaved oligonucleotide and/or uncleaved binding reagent from the reaction mixture by contacting the reaction mixture with a binding surface (also referred to herein as a “secondary surface”). In embodiments, the method has increased specificity as compared to a method that does not remove second cleaved oligonucleotide and/or uncleaved binding reagent from the reaction mixture. Binding surfaces are further described herein.

In embodiments, the method comprises detecting the first cleaved oligonucleotide(s) following removal of the second cleaved oligonucleotide and/or uncleaved binding reagent. In embodiments, the detecting comprises contacting the reaction mixture with a detection surface comprising a targeting agent, thereby immobilizing the first cleaved oligonucleotide(s) to the detection surface via hybridization of the targeting agent on the detection surface and the TAC on the first cleaved oligonucleotide. In embodiments, the detecting further comprises binding the amplification primer to a template oligonucleotide and extending the amplification primer to form an extended sequence. In embodiments, the extending comprises polymerase chain reaction (PCR), ligase chain reaction (LCR), strand displacement amplification (SDA), self-sustained synthetic reaction (3SR), or an isothermal amplification method. In embodiments, the extending comprises an isothermal amplification method. In embodiments, the isothermal amplification method is RCA. In embodiments, the extended sequence binds an anchoring reagent immobilized on the detection surface. In embodiments, the coronavirus nucleic acid is detected and/or quantified by detecting or quantifying the amount of extended sequence bound to the detection surface as described herein. In embodiments, the surface is contacted with a labeled probe that binds to the extended sequence, wherein the labeled probe comprises a detectable label. In embodiments, the detectable label comprises an ECL label. Additional exemplary detectable labels are provided herein. In embodiments, the coronavirus nucleic acid is SARS-CoV-2 RNA.

In embodiments, the first cleaved oligonucleotide remains bound to the coronavirus nucleic acid. In embodiments, the method further comprises amplifying the coronavirus nucleic acid to form one or more additional copies of the coronavirus nucleic acid, forming a plurality of binding complexes with each copy of the coronavirus nucleic acid, and detecting the plurality of binding complexes, thereby detecting the coronavirus in the biological sample. In embodiments, the method comprises amplifying the coronavirus nucleic acid via the amplification primer on the first cleaved oligonucleotide. In embodiments, the amplified coronavirus nucleic acid is contacted with an additional copy of the binding reagent, the binding complex formed therefrom is contacted with the RNA-guided nickase to cleave the binding reagent, and further amplifying the amplified coronavirus nucleic acid, thereby forming one or more additional copies of the coronavirus nucleic acid. In embodiments, the method comprises forming a plurality of binding complexes with the one or more additional copies of the coronavirus nucleic acid. In embodiments, the method comprises removing the uncleaved binding reagent and second cleaved oligonucleotide as described herein. In embodiments, the method comprises detecting the plurality of binding complexes as described herein, thereby detecting the coronavirus in the biological sample. In embodiments, forming the additional copies of the coronavirus nucleic acid amplifies the assay signal. In embodiments, the method has increased sensitivity of coronavirus detection as compared to a method that does not amplify the assay signal as described herein. In embodiments, the method is capable of detecting a lower amount of coronavirus nucleic acid in a biological sample as compared with a method that does not form the one or more additional coronavirus nucleic acids and the plurality of binding complexes, as described herein. In embodiments, the coronavirus nucleic acid is SARS-CoV-2 RNA.

### Assay Embodiment II. Collateral Cleavage, Extension and Detection

In embodiments, the invention provides a method for detecting a nucleic acid of interest in a sample, comprising: (a) contacting the sample with a site-specific nuclease comprising collateral cleavage activity and an oligonucleotide detection reagent, wherein the oligonucleotide detection reagent comprises: (i) a targeting agent complement (TAC); (ii) an amplification primer; and (iii) an amplification blocker, wherein the site-specific nuclease binds to the nucleic acid of interest and collaterally cleaves the oligonucleotide detection reagent to remove the amplification blocker therefrom, thereby generating a first cleaved oligonucleotide comprising the TAC and the amplification primer; (b) immobilizing the first cleaved oligonucleotide to a detection surface comprising a targeting agent, wherein the targeting agent is a binding partner of the TAC; (c) extending the first cleaved oligonucleotide to form an extended oligonucleotide; and (d) detecting the extended oligonucleotide, thereby detecting the nucleic acid of interest in the sample.

In embodiments, the nucleic acid of interest is a single-stranded oligonucleotide. In embodiments, the nucleic acid of interest is DNA, e.g., single-stranded DNA (ssDNA). In embodiments, the nucleic acid of interest is RNA, e.g., single-stranded RNA (ssRNA). Exemplary nucleic acids of interest and samples are provided herein. In embodiments, the sample comprising the nucleic acid of interest is contacted with a site-specific nuclease. In embodiments, the site-specific nuclease binds to the nucleic acid of interest. In embodiments, the site-specific nuclease is a Cas nuclease. Cas nucleases are further described, e.g., in Anzalone et al., Nat Biotechnol 38:824-844 (2020); Makarova et al., Methods Mol Biol 1311:41-75 (2015); Jinek et al., Science 343:1247997 (2014); Mali et al., Science 339(6121):823-826 (2013); Mali et al., Nat Method 10:957-963 (2013).

In embodiments, the Cas nuclease has collateral nuclease activity. As used herein, “collateral nuclease activity” means that the nuclease, following recognition and cleavage of a target nucleic acid, non-specifically cleaves any nearby nucleic acid (e.g., ssDNA or RNA) regardless of the sequence of the nearby nucleic acid. In the context of Cas nucleases, collateral nuclease activity refers to the Cas nuclease non-specifically cleaving any nearby nucleic acid, regardless of the nearby nucleic acid's complementarity to the guide RNA. Collateral nuclease activity of Cas nucleases is further described in, e.g., Gootenberg et al., Science 356(6336):438-442 (2017); Chen et al., Science 360(6387):436-439 (2018); and Li et al., ACS Synth Biol 8(10):2228-2237 (2019). In embodiments, the Cas nuclease is capable of collaterally cleaving a single-stranded oligonucleotide, e.g., RNA or ssDNA.

In embodiments, the nucleic acid of interest is RNA. In embodiments, the oligonucleotide detection reagent is RNA. In embodiments, the site-specific nuclease is a Cas13 nuclease. In embodiments, the Cas13 has collateral nuclease activity. In embodiments, the Cas13 is capable of collaterally cleaving RNA. Collateral nuclease activity is described herein. In embodiments, the Cas13 is Cas13a, Cas13b, Cas13c, or Cas13d. In embodiments, the Cas13 is LwaCas13a, CcaCas13b, LbaCas13a, or PsmCas13b, as described in Table 3. Further non-limiting examples of Cas13 proteins are provided in Table 3. Cas13 proteins are further described in, e.g., Abudayyeh et al., Science 353(6299):aaf5573 (2016); Cox et al., Science 358(6366):1019-1027 (2017); O'Connell, J Mol Biol 431(1):66-87 (2019).

TABLE 3
Exemplary Cas13 Nucleases

| Bacterial Species | Protein Name |
|---|---|
| Leptotrichia wadeii | LwaCas13a |
| Leptotrichia buccalis | LbuCas13a |
| Capnocytophaga canimorsus | CcaCas13b |
| Cc5 (NC_015846.1) | |
| Lachnospiraceae bacterium NK4A179 | LbaCas13a |
| Prevotella sp. MA2016 | PsmCas13b |
| (NZ_JHUW01000010.1) | |
| Prevotella sp. P5-125 | PspCas13b |
| Ruminococcus flavefaciens | RfxCas13d |
| Prevotella sp. P5-125 (NZ_JXQL01000055.1) | PspCas13b |
| Alistipes sp. ZOR0009 (NZ_JTLD01000029.1) | AspCas13b |
| Bergeyella TCC 43767 | BzoCas13b |
| (AG zoohelcum YA01000037.1) | |
| Riemerella anatipestifer | RanCas13b |
| Prevotella saccharolytica JCM | PsaCas13b |
| 17484 (NZ_BAKN01000001.1) | |
| Prevotella buccae ATCC 33574 (NZ_GL5863) | PbuCas13b |
| Prevotella intermedia ATCC 25611 | PinCas13b |
| (NZ_JAEZ01000017.1) | |
| Prevotella intermedia (NZ_LBGT01000010.1) | Pin2Cas13b |
| Prevotella intermedia (NZ_AP014926.1) | Pin3Cas13b |
| Prevotella aurantiaca JCM 15754 | PauCas13b |
| (NZ_BAKF01000019.1) | |
| Porphyromonas gulae (NZ_JRAL01000022.1) | PguCas13b |
| Porphyromonas gingivalis (NZ_CP0AJW4) | PgiCas13b |

In embodiments, the nucleic acid of interest is ssDNA. In embodiments, the oligonucleotide detection reagent is ssDNA. In embodiments, the site-specific nuclease is a Cas12 nuclease (also known as Cpf1 nuclease). In embodiments, the Cas12 has collateral nuclease activity. In embodiments, the Cas12 is capable of collaterally cleaving ssDNA. Collateral nuclease activity is described herein. In embodiments, the Cas12 is Cas12a or Cas12b. In embodiments, the Cas12 is LbaCas12a, AsCas12, FnCas12a or AaCas12b, as described in Table 2. Cas12 proteins are further described in, e.g., Makarova et al., The CRISPR Journal 1(5):325-333 (2018). In embodiments, the Cas12 is a Cas12 nuclease as described in Table 2.

In embodiments where the site-specific nuclease is Cas12 and the oligonucleotide detection reagent is ssDNA, each of the TAC and the amplification primer further comprises a nuclease-resistant nucleotide. In embodiments, the nuclease-resistant nucleotide prevents cleavage of the TAC and the amplification primer by the site-specific nuclease. Nucleotide modifications that confer resistance to nuclease cleavage are further described, e.g., in Kawasaki et al., J Med Chem 36:831-841 (1993) and Allerson et al., J Med Chem 48:901-904 (2005). In embodiments, the nuclease-resistant nucleotide comprises a 2′-O-Methyl (2′OMe) moiety, a 2′-O-(2-Methoxyethyl) (2′MOE) moiety, a locked nucleic acid (LNA), a phosphorothioate linkage, a 2′-fluoro moiety, or a combination thereof.

In embodiments, the site-specific nuclease, e.g., Cas13 or Cas12, is an RNA-guided nuclease. In embodiments, the site-specific nuclease forms a complex with a guide polynucleotide, e.g., a guide RNA. As described herein, the guide RNA is a nucleic acid comprising a tracrRNA and a crRNA, which is complementary to a target sequence (e.g., nucleic acid of interest). In embodiments, the guide RNA is a single guide RNA (sgRNA) comprising both the tracrRNA and the crRNA. In embodiments, the guide RNA comprises the tracrRNA and the crRNA on separate polynucleotides. Methods of designing and making guide RNA to form a complex with a Cas13 or Cas12 nuclease are known in the field and described, e.g., in Bandaru et al., Sci Rep 10:11610 (2020); Wessels et al., Nat Biotechnol 38:722-727 (2020); Gootenberg et al., Science 356:438-442 (2017); and Gootenberg et al., Science. 360:439-444 (2018). In embodiments, the guide polynucleotide comprises a complementary sequence to the nucleic acid of interest. In embodiments, the site-specific nuclease, e.g., Cas13 or Cas12, binds to the nucleic acid of interest via complementarity between the guide polynucleotide and the nucleic acid of interest. In embodiments, the nucleic acid of interest is a polynucleotide in a sample, wherein the entire polynucleotide binds to the site-specific nuclease, e.g., Cas13 or Cas12. In embodiments, the nucleic acid of interest is a portion or region of another compound, e.g., a longer polynucleotide, wherein a portion of the longer polynucleotide does not bind to the site-specific nuclease, e.g., Cas13 or Cas12. In embodiments, the site-specific nuclease, e.g., Cas13 or Cas12, cleaves the nucleic acid of interest following the binding. In embodiments, binding and/or cleavage of the site-specific nuclease, e.g., Cas13 or Cas12, to the nucleic acid of interest activates the collateral nuclease activity of the site-specific nuclease.

In embodiments, the oligonucleotide detection reagent comprises a single-stranded oligonucleotide. In embodiments, the oligonucleotide detection reagent is capable of being cleaved by the collateral nuclease activity of the site-specific nuclease, e.g., Cas13 or Cas12. In embodiments, the method comprises contacting the sample comprising the nucleic acid of interest with: (i) the site-specific nuclease and (ii) the oligonucleotide detection reagent, wherein the site-specific nuclease (1) binds to and/or cleaves the nucleic acid of interest as described herein and (2) collaterally cleaves the oligonucleotide detection reagent. In embodiments, the sample is simultaneously or substantially simultaneously contacted with (i) the site-specific nuclease and (ii) the oligonucleotide detection reagent. As used herein, the term “simultaneous” in reference to one or more events (e.g., contacting the sample with the site-specific nuclease and the oligonucleotide detection reagent) means that the events occur at exactly the same time or at substantially the same time, e.g., simultaneous events described herein can occur less than or about 10 minutes apart, less than or about 5 minutes apart, less than or about 2 minutes apart, less than or about 1 minute apart, less than or about 30 seconds apart, less than or about 15 seconds apart, or less than or about 5 seconds apart. In embodiments, the sample is contacted with the site-specific nuclease prior to being contacted with the oligonucleotide detection reagent.

In embodiments, the oligonucleotide detection reagent comprises a TAC, an amplification primer, and an amplification blocker. In embodiments, the oligonucleotide detection reagent comprises, in 5′ to 3′ order, the TAC, the amplification primer, and the amplification blocker. In embodiments, the oligonucleotide detection reagent comprises, in 3′ to 5′ order, the TAC, the amplification primer, and the amplification blocker. In embodiments, collateral cleavage of the oligonucleotide detection reagent removes the amplification blocker from the oligonucleotide detection reagent, thereby generating a first cleaved oligonucleotide comprising the TAC and the amplification primer.

In embodiments, the oligonucleotide detection reagent is about 20 to about 300, about 25 to about 280, about 30 to about 250, about 35 to about 220, about 40 to about 200, about 45 to about 180, about 50 to about 150, about 55 to about 120, about 60 to about 100, or about 65 to about 80 nucleotides in length.

In embodiments, the amplification primer comprises a primer for polymerase chain reaction (PCR), ligase chain reaction (LCR), strand displacement amplification (SDA), self-sustained synthetic reaction (3SR), or an isothermal amplification method. In embodiments, the amplification primer comprises a primer for an isothermal amplification method. In embodiments, the isothermal amplification method is helicase-dependent amplification. In embodiments, the isothermal amplification method is rolling circle amplification (RCA). Amplification methods, including RCA, are further described herein. In embodiments, the amplification primer is about 1 to about 50, about 5 to about 45, about 10 to about 40, about 12 to about 35, about 15 to about 30, about 18 to about 40, about 20 to about 35, about 25 to about 30, or about 30 to about 35 nucleotides in length. In embodiments, the amplification primer is about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In embodiments, the amplification primer comprises the sequence GACAGAACTAGACAC (SEQ ID NO:64).

In embodiments, the amplification blocker blocks amplification of the amplification primer. In embodiments, the amplification blocker comprises an oligonucleotide that blocks amplification of the amplification primer by preventing polymerase binding, inhibiting polymerase activity, and/or promoting polymer dissociation from the amplification primer. In embodiments, the amplification blocker comprises a nucleotide modification. Non-limiting examples of nucleotide modifications that block amplification include 3′-spacer C3, 3′-phosphate, 3′-dideoxy cytidine (3′-ddC), and 3′-inverted end. In embodiments, the amplification blocker comprises a PNA and/or an LNA. In embodiments, the amplification blocker comprises a 2′-O-methyl uridine, a 3′-inverted dT, a digoxigenin, a biotin, or a combination thereof. In embodiments, the amplification blocker comprises a secondary structure, e.g., a stem loop or a pseudoknot.

In embodiments, the oligonucleotide detection reagent further comprises a nuclease cleavage site. In embodiments, the nuclease cleavage site comprises a sequence at which the site-specific nuclease preferentially cleaves during collateral cleavage. In embodiments, the nuclease cleavage site comprises a poly ribouridine (rU) sequence. In embodiments, the poly rU sequence comprises at least or about 2, at least or about 3, at least or about 4, at least or about 5, at least or about 6, at least or about 7, at least or about 8, at least or about 9, or at least or about 10 rU nucleotides. In embodiments, the nuclease cleavage site comprises an RNA dinucleotide. Preferred RNA dinucleotides for cleavage by Cas13 and Cas12 are described in, e.g., Slaymaker et al., Cell Rep 26(13):3741-3751.e5 (2019); East-Seletsky et al., Mol Cell 66(3):373-383.e3 (2017); Gootenberg et al., Science 360(6387):439-444 (2018). For example, the preferred RNA dinucleotide for LwaCas13a, CcaCas13b, LbaCas13a, and PsmCas13b are AU, UC, AC, and GA, respectively.

In embodiments, the nuclease cleavage site is positioned between the amplification primer and the amplification blocker. Thus, in embodiments, the oligonucleotide detection reagent comprises, in 5′ to 3′ order, the TAC, the amplification primer, the nuclease cleavage site, and the amplification blocker. In embodiments, the site-specific nuclease cleavages the oligonucleotide detection reagent at the nuclease cleavage site, thereby generating the first cleaved oligonucleotide comprising the TAC and the amplification primer.

In embodiments, the TAC is a binding partner of a targeting agent on a detection surface. In embodiments, the TAC and the targeting agent comprise a binding pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the targeting agent and the TAC are cross-reactive moieties, e.g., thiol and maleimide or iodoacetamide; aldehyde and hydrazide; or azide and alkyne or cycloalkyne. In embodiments, the TAC and the targeting agent comprise complementary oligonucleotides. In embodiments, the TAC and the targeting agent are at least 90%, at least 95%, at least 98%, at least 99%, or 100% complementary. In embodiments, the TAC is an oligonucleotide of about 5 to about 100, about 6 to about 90, about 7 to about 80, about 8 to about 70, about 9 to about 60 nucleotides, about 10 to about 50, about 15 to about 45, about 20 to about 40, about 20 to about 30, about 20 to about 35, or about 30 to about 35 nucleotides in length. In embodiments, the TAC comprises any of SEQ ID NOs:68-71. In embodiments, the TAC comprises biotin, and the targeting agent comprises avidin or streptavidin. In embodiments, the method comprises immobilizing the first cleaved oligonucleotide to the detection surface via binding of the TAC to the targeting agent. Detection surfaces are further described herein.

In embodiments, the oligonucleotide detection reagent further comprises a secondary targeting agent complement (secondary TAC). In embodiments, the secondary TAC is a binding partner of a secondary targeting agent on a binding surface. In embodiments, the secondary TAC and the secondary targeting gent comprise a binding pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the secondary targeting agent and secondary TAC are cross-reactive moieties, e.g., thiol and maleimide or iodoacetamide; aldehyde and hydrazide; or azide and alkyne or cycloalkyne. In embodiments, the TAC and the targeting agent are substantially unreactive with the secondary TAC and the secondary targeting agent. In embodiments, the TAC and the targeting agent comprise biotin and avidin/streptavidin, and the secondary TAC and the secondary targeting agent comprise complementary oligonucleotides. In embodiments, the TAC and the targeting agent comprise complementary oligonucleotides, and the secondary TAC and the secondary targeting agent comprise complementary oligonucleotides that are substantially non-hybridizable to the TAC or targeting agent. In embodiments, the secondary TAC is an oligonucleotide of about 5 to about 100, about 6 to about 90, about 7 to about 80, about 8 to about 70, about 9 to about 60 nucleotides, about 10 to about 50, about 15 to about 45, about 20 to about 40, about 20 to about 30, or about 30 to about 35 nucleotides in length. In embodiments, the amplification blocker is a binding partner of the secondary targeting agent on the binding surface. In embodiments, the secondary TAC and/or the amplification blocker comprises digoxigenin, and the secondary targeting agent comprises an anti-digoxigenin antibody.

In embodiments, the TAC and the secondary TAC are on separate ends of the oligonucleotide detection reagent. In embodiments, the TAC is at a 5′ end of the oligonucleotide detection reagent, and the secondary TAC is at a 3′ end of the oligonucleotide detection reagent. In embodiments, the TAC is at a 3′ end of the oligonucleotide detection reagent, and the secondary TAC is at a 5′ end of the oligonucleotide detection reagent. In embodiments, the oligonucleotide detection reagent comprises, in 5′ to 3′ order: the TAC, the amplification primer, the amplification blocker, and the secondary TAC. In embodiments where the oligonucleotide detection reagent comprises a nuclease cleavage site, the oligonucleotide detection reagent comprises, in 5′ to 3′ order: the TAC, the amplification primer, the nuclease cleavage site, the amplification blocker, and the secondary TAC. In embodiments, the oligonucleotide binding reagent comprises, in 3′ to 5′ order: the TAC, the amplification primer, the hybridization region, the amplification blocker, and the secondary TAC. In embodiments, the secondary TAC is positioned adjacent to the amplification blocker on the oligonucleotide detection reagent, such that cleavage of the oligonucleotide detection reagent by the site-specific nuclease (e.g., at the nuclease cleavage site) forms (i) the first cleaved oligonucleotide described herein and (ii) a second cleaved oligonucleotide comprising the amplification blocker and the secondary TAC.

In embodiments, a reaction mixture comprising the first cleaved oligonucleotide, second cleaved oligonucleotide, and uncleaved oligonucleotide detection reagent is formed following contacting the sample with the site-specific nuclease and the oligonucleotide detection reagent. In embodiments, the presence of uncleaved oligonucleotide detection reagent and/or second cleaved oligonucleotide interferes with one or more of the downstream steps of the method, e.g., immobilization, extension, and/or detection of the first cleaved oligonucleotide. In embodiments, the method further comprises, prior to the extending of the first cleaved oligonucleotide, removing the second cleaved oligonucleotide, uncleaved oligonucleotide detection reagent, or both. In embodiments, the removing comprises contacting the reaction mixture (comprising the first cleaved oligonucleotide, second cleaved oligonucleotide, and uncleaved oligonucleotide detection reagent) with the binding surface comprising the secondary targeting agent described herein, wherein the second cleaved oligonucleotide and the uncleaved oligonucleotide detection reagent bind to the binding surface. In embodiments, the removing further comprises separating the binding surface comprising the second cleaved oligonucleotide and the uncleaved oligonucleotide detection reagent from the reaction mixture. In embodiments, the first cleaved oligonucleotide does not comprise the secondary TAC and therefore does not bind to the binding surface. In embodiments, the binding of the second cleaved oligonucleotide and the uncleaved oligonucleotide binding reagent to the binding surface reduces or eliminates interference with the immobilization, extension, and/or detection of the first cleaved oligonucleotide. In embodiments, the method has increased specificity as compared to a method that does not remove the second cleaved oligonucleotide and/or the uncleaved oligonucleotide detection reagent from the reaction mixture.

In embodiments, the method comprises, following removal of the second cleaved oligonucleotide and/or uncleaved oligonucleotide detection reagent, immobilizing the first cleaved oligonucleotide to the detection surface via binding of the TAC to the targeting agent. Immobilization of the first cleaved oligonucleotide is described herein.

In embodiments, the method comprises extending the immobilized first cleaved oligonucleotide on the detection surface to form an extended oligonucleotide. In embodiments, the extending comprises binding the amplification primer of the first cleaved oligonucleotide to a template oligonucleotide, and extending the amplification primer to form an extended oligonucleotide. In embodiments, the extending comprises polymerase chain reaction (PCR), ligase chain reaction (LCR), strand displacement amplification (SDA), self-sustained synthetic reaction (3SR), or an isothermal amplification method. In embodiments, the amplification primer comprises a primer for an isothermal amplification method. In embodiments, the isothermal amplification method is helicase-dependent amplification. In embodiments, the isothermal amplification method is rolling circle amplification (RCA). In embodiments, the template oligonucleotide for RCA comprises the sequence

(SEQ ID NO: 65)
GTTCTGTCATATTTCAGTGAATGCGAGTCCGTCTAAGAGAGTAGTACAG
CAAGAGTGTCTA.

In embodiments, the detection surface further comprises an anchoring reagent immobilized thereon. In embodiments, the extended oligonucleotide comprises an anchoring region. In embodiments, the extended oligonucleotide binds to the anchoring reagent via the anchoring region. In embodiments, the anchoring reagent comprises an oligonucleotide, aptamer, aptamer ligand, antibody, antigen, ligand, receptor, hapten, epitope, or mimotope. In embodiments, the anchoring reagent comprises a single-stranded oligonucleotide. In embodiments, the anchoring reagent comprises a double-stranded oligonucleotide. In embodiments, the anchoring reagent and the anchoring region comprise complementary oligonucleotides. In embodiments, the anchoring reagent comprises the sequence

(SEQ ID NO: 66)
AAGAGAGTAGTACAGCAGCCGTCAA.

In embodiments, binding the anchoring region to the anchoring reagent comprises forming a triple helix between the anchoring reagent and the extended oligonucleotide. In embodiments, binding the extended oligonucleotide to the anchoring reagent comprises: denaturing the anchoring region to expose a single-stranded region prior to the binding; exposing the anchoring to helicase activity prior to the binding; and/or exposing the anchoring region to nuclease treatment prior to the binding, wherein the anchoring region comprises one or more hapten-modified bases and the anchoring reagent comprises one or more antibodies specific for the hapten; and/or the anchoring region comprises one or more ligand-modified bases and the anchoring reagent comprises one or more receptors specific for the ligand.

In embodiments, the method comprises detecting the extended oligonucleotide. In embodiments, the detecting comprises measuring the amount of extended oligonucleotide bound to the detection surface. In embodiments, the nucleic acid of interest is detected and/or quantified by measuring the amount of extended oligonucleotide bound to the detection surface. In embodiments, the detecting comprises: contacting the extended oligonucleotide with a labeled probe comprising a detectable label, wherein the labeled probe binds to the extended oligonucleotide; and measuring the amount of labeled probe bound to the extended oligonucleotide. In embodiments, the labeled probe and the extended oligonucleotide comprise complementary oligonucleotides. In embodiments, the labeled probe comprises the sequence

(SEQ ID NO: 67)
CAGTGAATGCGAGTCCGTCTAAG.

In embodiments, the extended oligonucleotide comprises a modified base, and measuring the amount of extended oligonucleotide comprises contacting the extended oligonucleotide with a detectable moiety that binds to the modified base. In embodiments, the modified base comprises an aptamer, aptamer ligand, antibody, antigen, ligand, receptor, hapten, epitope, or mimotope, and the detectable moiety comprises a binding partner of the modified base and a detectable label. In embodiments, the modified base comprises streptavidin or avidin, and the detectable moiety comprises (i) biotin and (ii) a detectable label. In embodiments, the modified base comprises biotin, and the detectable moiety comprises (i) streptavidin or avidin and (ii) a detectable label. Methods of detecting extended oligonucleotides are further described herein.

In embodiments, the detectable label is detectable by light scattering, optical absorbance, fluorescence, chemiluminescence, electrochemiluminescence (ECL), bioluminescence, phosphorescence, radioactivity, magnetic field, or combinations thereof. In embodiments, the detectable label comprises phycoerythrin (PE). In embodiments, the detectable label comprises a β-galactosidase (β-gal) enzyme that can be detected by fluorescence detection when the β-gal enzyme cleaves a substrate such as resorufin-β-D-galactopyranoside to yield a fluorescent signal. In embodiments, the detectable label comprises an ECL label, and the detecting comprises measuring an ECL signal. In embodiments, the ECL label comprises ruthenium. ECL labels, ECL assays, and instrumentation for conducting ECL assays are further described herein.

In embodiments, the method comprises contacting the sample with: (i) the site-specific nuclease and (ii) multiple copies of the oligonucleotide detection reagent. In embodiments, the collateral nuclease activity of the site-specific nuclease cleaves the multiple copies of the oligonucleotide detection reagent, thereby generating a plurality of first cleaved oligonucleotides.

In embodiments, the method further comprises, following contacting the sample with: (i) the site-specific nuclease and (ii) a first copy of the oligonucleotide detection reagent as described herein, contacting the sample with one or more additional copies of the oligonucleotide detection reagent. In embodiments, the method further comprises contacting the sample with a second nuclease, wherein the second nuclease is activated upon cleavage of the first copy of the oligonucleotide detection reagent and cleaves the one or more additional copies of the oligonucleotide detection reagent, thereby generating a plurality of first cleaved oligonucleotides. In embodiments, the oligonucleotide detection reagent further comprises a second nuclease cleavage site. In embodiments, the second nuclease cleavage site is positioned between the amplification primer and the amplification blocker, wherein the second nuclease cleaves the one or more additional copies of the oligonucleotide detection reagent at the second nuclease cleavage site. In embodiments, the second nuclease is Csm6. Csm6 is a Type III CRISPR effector protein that is activated by the cleavage products of Cas13. Non-limiting examples of Csm6 proteins include EiCsm6, LsCsm6, and TtCsm6. See, e.g., Gootenberg et al., Science 360(6387):439-444 (2018). In embodiments, the second nuclease increases sensitivity of the method by cleaving additional copies of the oligonucleotide detection reagent to form a plurality of first cleaved oligonucleotides.

In embodiments, the method comprises immobilizing the plurality of first cleaved oligonucleotides to the detection surface; extending each of the immobilized plurality of first cleaved oligonucleotides to form a plurality of extended oligonucleotides; and detecting the plurality of extended oligonucleotides. In embodiments, the plurality of first cleaved oligonucleotides amplifies the assay signal. In embodiments, the method has increased sensitivity for detecting the nucleic acid of interest as compared to a method that does not form the plurality of first cleaved oligonucleotides as described herein. In embodiments, the method is capable of detecting a lower amount of nucleic acid of interest in a sample as compared with a method that does not form the plurality of first cleaved oligonucleotides as described herein.

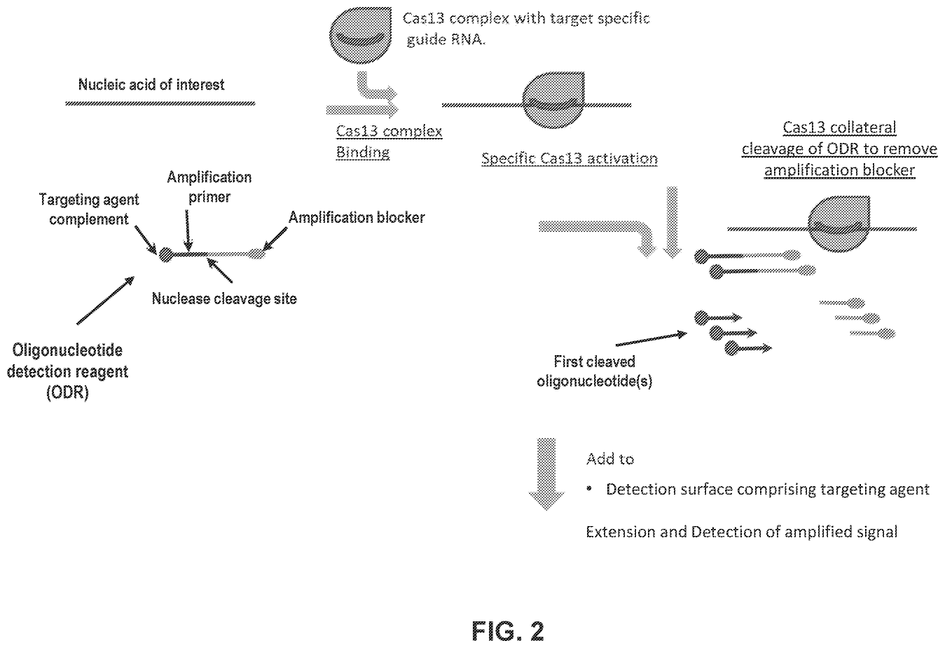

An embodiment of the method is illustrated in FIG. 2. In FIG. 2, an oligonucleotide detection reagent comprises, in 5′ to 3′ order, a TAC, an amplification primer, a nuclease cleavage site, and an amplification blocker. A Cas13 complex binds a nucleic acid of interest, thereby activating collateral cleavage activity of the Cas13. The Cas13 collaterally cleaves one or more copies of the oligonucleotide detection reagent, thereby generating one or more first cleaved oligonucleotides, each comprising the TAC and the amplification primer. The reaction mixture sample is incubated on a detection surface comprising a targeting agent to immobilize the one or more first cleaved oligonucleotides onto the detection surface. In embodiments, the immobilized first cleaved oligonucleotide(s) are subjected to extension and detection as described herein. In embodiments, the nucleic acid of interest is detected via detection of the immobilized first cleaved oligonucleotide(s).

An exemplary protocol for performing the method comprises:

1. Preparing the sample comprising the nucleic acid of interest. In embodiments, the preparing comprises extracting a nucleic acid (e.g., genomic DNA or RNA) from an organism of interest (e.g., a virus) that contains the nucleic acid of interest. In embodiments, the preparing further comprises producing cDNA from a genomic RNA, e.g., use reverse transcriptase. In embodiments, the preparing further comprises producing a target RNA from cDNA, e.g., using RNA polymerase.

2A. Incubating a sample reaction mixture, comprising the Cas enzyme (e.g., about 10 to about 100 nM, or about 20 to about 80 nM, or about 30 to about 60 nM, about 40 to 50 nM, or about 45 nM of purified Cas13), guide RNA targeting the nucleic acid of interest (e.g., about 5 to about 50 nM, about 10 to about 40 nM, about 20 to about 30 nM, about 22 to about 25 nM, or about 22.5 nM), the oligonucleotide detection reagent (e.g., about 0.05 to about 100 nM, about 0.1 to about 80 nM, about 0.2 nM to about 60 nM, about 0.3 nM to about 50 nM, about 0.4 nM to about 40 nM, about 0.1 to about 20 nM, or about 0.1 to about 10 nM), and the sample that comprises the nucleic acid of interest. In embodiments, the TAC of the oligonucleotide detection reagent is biotin. In embodiments, the sample reaction mixture is in an assay buffer of pH about 6 to about 8, about 6.5 to about 7.5, or about 6.7 to about 7. In embodiments, the sample reaction mixture comprises a reaction volume of about 10 μL to about 1 mL, about 20 μL to about 700 μL, about 50 μL to about 500 μL, about 70 μL to about 200 μL, about 90 to about 150 μL, or about 100 μL. In embodiments, the sample reaction mixture is incubated for about 10 minutes to about 6 hours, about 30 minutes to about 4 hours, or about 1 hour to about 3 hours. In embodiments, the sample reaction mixture is incubated at about 20° C. to about 50° C., about 25° C. to about 45° C., about 30° C. to about 40° C., or about 37° C. In embodiments, the sample reaction mixture is incubated for about 1 hour to about 3 hours at about 37° C.

2B. Preparing an assay plate. In embodiments, the preparing comprises coating an assay plate with a targeting agent and an anchoring reagent. In embodiments, the targeting agent is streptavidin. In embodiments, the assay plate is a 96-well plate. In embodiments, the assay plate is coated with about 100 to about 500 ng of streptavidin, about 150 to about 400 ng of streptavidin, about 200 to about 350 ng of streptavidin, about 250 to about 300 ng of streptavidin, or about 275 ng of streptavidin. In embodiments, the assay plate is coated with about 100 to about 900 nM anchoring reagent, about 200 to about 700 nM anchoring reagent, about 300 to about 500 nM anchoring reagent, or about 400 nM anchoring reagent. In embodiments, the assay plate is washed, e.g., with PBS, following the coating. In embodiments, the assay plate is blocked with a blocking solution following the coating and washing. In embodiments, the blocking solution reduces and/or eliminates non-specific binding to the streptavidin and/or anchoring reagent on the assay plate.

In embodiments, steps 1 and 2 are performed simultaneously or substantially simultaneously. In embodiments, the producing of cDNA from genomic RNA and/or the producing RNA from cDNA of step 1, and the incubating of step 2 are performed in the same reaction mixture.

3A. Incubating the sample reaction on the assay plate. In embodiments, about 10 to about 100 μL, about 20 to about 80 μL, about 30 to about 70 μL, about 40 to about 60 μL, or about 50 μL of the sample reaction is added to a well of the assay plate. In embodiments, the sample reaction is incubated for about 10 minutes to about 4 hours, about 30 minutes to about 2 hours, or about 1 hour. In embodiments, the sample reaction is incubated at about 15° C. to about 40° C., about 20° C. to about 37° C., about 25° C. to about 30° C., or about 27° C. In embodiments, the sample reaction is incubated for about 1 hour at about 27° C. In embodiments, the assay plate is washed, e.g., with PBS, following the incubating.

3B. Removing second cleaved oligonucleotide and/or uncleaved oligonucleotide detection reagent. In embodiments, the removing comprises contacting the sample reaction with magnetic beads comprising a secondary targeting agent. In embodiments, the secondary targeting agent is a binding partner of a secondary TAC and/or an amplification blocker on the oligonucleotide detection reagent. In embodiments, the magnetic beads are incubated with the sample reaction for about 10 minutes to about 4 hours, about 30 minutes to about 2 hours, or about 1 hour. In embodiments, the magnetic beads are incubated with the sample reaction at about 20° C. to about 50° C., about 25° C. to about 45° C., about 30° C. to about 40° C., or about 37° C. In embodiments, the magnetic beads are incubated with the sample reaction for about 1 hour at about 37° C. In embodiments, following the incubation, the beads are removed or separated (e.g., concentrated on a side of the sample reaction container such that the beads are no longer in contact with the sample reaction) from the sample reaction by contacting the sample reaction container with a magnet.

4. Performing an RCA reaction. RCA reactions are described, e.g., in U.S. Pat. No. 10,114,015. In embodiments, the RCA reaction comprises adding a ligation mix comprising ligase (e.g., T4 DNA ligase), ATP, template oligonucleotide, and ligation buffer to the sample reaction in the assay plate well. In embodiments, the sample reaction is incubated with the ligation mix for about 10 minutes to about 2 hours, about 20 minutes to about 1 hour, or about 30 minutes. In embodiments, the sample reaction is incubated with the ligation mix at about 15° C. to about 40° C., about 20° C. to about 37° C., about 25° C. to about 30° C., or about 22° C. to about 28° C. embodiments, the sample reaction is incubated for about 30 minutes at room temperature, e.g., about 22° C. to about 28° C. In embodiments, following incubation of the sample reaction and the ligation mix, a polymerase mix, comprising dNTPs (about 100 to about 500 μM, about 200 to about 400 μM, or about 250 μM of each of dATP, dGTP, dCTP), DNA polymerase (e.g., Phi29 DNA polymerase), and a labeled probe (e.g., about 1 to about 10 nM, about 2 to about 9 nM, about 4 to about 8 nM, about 6 to about 7 nM, about 5 nM, about 6 nM, or about 7 nM) as described herein, is added to the assay plate well to perform the RCA reaction. RCA reaction conditions are known in the art. In embodiments, the assay plate is washed, e.g., with PBS buffer, following the RCA reaction.

5. Reading the plate. In embodiments, about 50 to about 500 μL, about 100 to about 300 μL, or about 150 μL of a read buffer is added to the assay plate well. In embodiments, the assay plate is read on a plate reader immediately or substantially immediately following addition of the read buffer.

Virus Detection

In embodiments, the methods provided herein are used to detect a virus in a sample. In embodiments, the method detects a viral nucleic acid. In embodiments, the viral nucleic acid is viral DNA or viral RNA. In embodiments, the method is used to diagnose a viral infection in a subject. In embodiments, the virus is a respiratory virus, e.g., influenza A (FluA), influenza B (FluB), respiratory syncytial virus (RSV), a coronavirus, or a combination thereof. In embodiments, the virus is a coronavirus. In embodiments, the coronavirus is SARS-CoV-2. In embodiments, the nucleic acid of interest is a coronavirus nucleic acid.

In embodiments, the invention provides a method for detecting a coronavirus nucleic acid in a biological sample, comprising: a) contacting the biological sample with a site-specific nuclease comprising collateral cleavage activity and a binding reagent, wherein the site-specific nuclease binds to the coronavirus nucleic acid and cleaves the binding reagent; b) immobilizing the cleaved binding reagent onto a detection surface; and c) detecting the immobilized cleaved binding reagent, thereby detecting the coronavirus in the biological sample. In embodiments, the coronavirus nucleic acid is RNA. In embodiments, the binding reagent is an oligonucleotide detection reagent described herein.

In embodiments, the binding reagent comprises one or more of: a TAC, an amplification primer, a nuclease cleavage site (also referred to herein as a “ribonuclease recognition site”), an amplification blocker, and a secondary TAC. In embodiments, the binding reagent is an RNA oligonucleotide comprising, in 5′ to 3′ order: a TAC, an amplification primer, a ribonuclease recognition site, and an amplification blocker. In embodiments, the binding reagent is an RNA oligonucleotide comprising, in 5′ to 3′ order: a TAC, an amplification primer, a ribonuclease recognition site, an amplification blocker, and a secondary TAC. TACs, amplification primers, nuclease cleavage sites, amplification blockers, and secondary TACs of oligonucleotide detection reagents are described herein.

In embodiments, the method comprises contacting the biological sample with a RNA-guided ribonuclease. In embodiments, the RNA-guided ribonuclease is Cas13. In embodiments, the Cas13 is Cas13a, Cas13b, Cas13c, or Cas13d. Cas13 nucleases are further described herein.

In embodiments, the RNA-guided ribonuclease forms a complex with a guide RNA that hybridizes to a target coronavirus nucleic acid (i.e., the ribonuclease is “guided” to the target coronavirus nucleic acid). In embodiments, the RNA-guided ribonuclease cleaves the coronavirus nucleic acid. In embodiments, the binding reagent is added to the reaction mixture containing the RNA-guided ribonuclease and coronavirus nucleic acid after binding and cleavage of the coronavirus nucleic acid by the RNA-guided ribonuclease. In embodiments, the binding reagent is added to the reaction mixture containing the RNA-guided ribonuclease and coronavirus nucleic acid simultaneously or substantially simultaneously as binding and cleavage of the coronavirus nucleic acid by the RNA-guided ribonuclease. In embodiments, the coronavirus nucleic acid is SARS-CoV-2 RNA.

In embodiments, the RNA-guided ribonuclease cleaves the binding reagent after binding and cleaving the coronavirus nucleic acid. In embodiments, the RNA-guided ribonuclease cleaves the binding reagent at the ribonuclease recognition site, thereby removing the amplification blocker from the binding reagent to generate a first cleaved oligonucleotide (also referred to herein as a “cleaved binding reagent”) comprising the amplification primer and TAC. In embodiments, the RNA-guided ribonuclease cleaves the binding reagent to form a second cleaved oligonucleotide comprising the amplification blocker and the secondary TAC (also referred to herein as a “cleaved amplification blocker-secondary targeting agent”). In embodiments, the coronavirus nucleic acid is SARS-CoV-2 RNA.

In embodiments, the method further comprises contacting the binding reagent with a second ribonuclease, wherein the second ribonuclease is activated upon cleavage of the binding reagent and cleaves additional copies of the binding reagent. In embodiments, the binding reagent further comprises a second nuclease cleavage site (also referred to herein as a “second ribonuclease recognition site”). In embodiments, the second ribonuclease is Csm6. Csm6 is further described herein. In embodiments, the second ribonuclease increases sensitivity of the method by increasing cleavage of the binding reagent to remove amplification blocker, thereby enabling amplification of the coronavirus nucleic acid. In embodiments, the coronavirus nucleic acid is SARS-CoV-2 RNA.

In embodiments, following the amplifying, a reaction mixture containing the first cleaved oligonucleotide, uncleaved binding reagent, and second cleaved oligonucleotide is formed. In embodiments, the method comprises removing the second cleaved oligonucleotide and/or uncleaved binding reagent from the reaction mixture by contacting the reaction mixture with the binding surface (also referred to herein as a “secondary surface”). In embodiments, the method has increased specificity as compared to a method that does not remove the second cleaved oligonucleotide and/or uncleaved binding reagent from the reaction mixture. Binding surfaces are further described herein.

In embodiments, the method comprises detecting the first cleaved oligonucleotide. In embodiments, the detecting is performed after removal of the second cleaved oligonucleotide and/or uncleaved binding reagent. In embodiments, the detecting comprises contacting the reaction mixture with a detection surface comprising a targeting agent, thereby immobilizing the first cleaved oligonucleotide to the detection surface via hybridization of the targeting agent on the surface and the TAC on the binding reagent. In embodiments, the detecting further comprises binding the amplification primer to a template oligonucleotide and extending the amplification primer to form an extended sequence. In embodiments, the extending comprises polymerase chain reaction (PCR), ligase chain reaction (LCR), strand displacement amplification (SDA), self-sustained synthetic reaction (3SR), or an isothermal amplification method. In embodiments, the extending comprises an isothermal amplification method. In embodiments, the isothermal amplification method is RCA. In embodiments, the extended sequence binds an anchoring reagent immobilized on the surface. In embodiments, the coronavirus nucleic acid is detected and/or quantified by detecting or quantifying the amount of extended sequence bound to the surface as described herein. In embodiments, the surface is contacted with a labeled probe that binds to the extended sequence, wherein the labeled probe comprises a detectable label. In embodiments, the detectable label comprises an ECL label. Additional exemplary detectable labels are provided herein. In embodiments, the coronavirus nucleic acid is SARS-CoV-2 RNA.

### Assay Embodiment III. Collateral Cleavage, Blocker Separation and Detection

In embodiments, the invention provides a method for detecting a nucleic acid of interest in a sample, comprising: (a) contacting the sample with a site-specific nuclease comprising collateral cleavage activity and an oligonucleotide detection reagent, wherein the oligonucleotide detection reagent comprises: (i) a primary targeting agent complement (primary TAC); (ii) a secondary targeting agent complement (secondary TAC); and (iii) a detectable label; wherein the site-specific nuclease binds to the nucleic acid of interest and collaterally cleaves the oligonucleotide detection reagent, thereby generating (i) a cleaved secondary TAC and (ii) a first cleaved oligonucleotide comprising the primary TAC and the detectable label; (b) binding the cleaved secondary TAC, uncleaved oligonucleotide detection reagent, or both, to a binding surface comprising a secondary targeting agent that is a binding partner of the secondary TAC; (c) immobilizing the first cleaved oligonucleotide to a detection surface comprising a primary targeting agent, wherein the primary targeting agent is a binding partner of the primary TAC; and (d) detecting the first cleaved oligonucleotide bound to the detection surface, wherein the secondary TAC and the uncleaved oligonucleotide detection reagent on the binding surface are substantially undetected, thereby detecting the nucleic acid of interest in the sample.

In embodiments, the nucleic acid of interest is a single-stranded oligonucleotide. In embodiments, the nucleic acid of interest is DNA, e.g., single-stranded DNA (ssDNA). In embodiments, the nucleic acid of interest is RNA, e.g., single-stranded RNA (ssRNA). Exemplary nucleic acids of interest and samples are provided herein.

In embodiments, the sample comprising the nucleic acid of interest is contacted with a site-specific nuclease. In embodiments, the site-specific nuclease binds to the nucleic acid of interest. In embodiments, the site-specific nuclease is a Cas nuclease. Cas nucleases are further described herein. In embodiments, the Cas nuclease has collateral nuclease activity. Collateral nuclease activity, e.g., of Cas nucleases, is further described herein. In embodiments, the Cas nuclease is capable of collaterally cleaving a single-stranded oligonucleotide, e.g., RNA or ssDNA.

In embodiments, the nucleic acid of interest is RNA. In embodiments, oligonucleotide detection reagent is RNA. In embodiments, the site-specific nuclease is a Cas13 nuclease, as described herein. In embodiments, the Cas13 has collateral nuclease activity. In embodiments, the Cas13 is capable of collaterally cleaving RNA. In embodiments, the Cas13 is Cas13a, Cas13b, Cas13c, or Cas13d. In embodiments, the Cas13 is LwaCas13a, CcaCas13b, LbaCas13a, or PsmCas13b. In embodiments, the Cas13 is a Cas13 nuclease as described in Table 3.

In embodiments, the nucleic acid of interest is ssDNA. In embodiments, the oligonucleotide detection reagent is ssDNA. In embodiments, the site-specific nuclease is a Cas12 nuclease, as described herein. In embodiments, the Cas12 has collateral nuclease activity. In embodiments, the Cas12 is capable of collaterally cleaving ssDNA. In embodiments, the Cas12 is Cas12a or Cas12b. In embodiments, the Cas12 is LbaCas12a, AsCas12, FnCas12a or AaCas12b. In embodiments, the Cas12 is a Cas12 nuclease as described in Table 2.

In embodiments where the site-specific nuclease is Cas12 and the oligonucleotide detection reagent is ssDNA, each of the primary TAC and the amplification primer further comprises a nuclease-resistant nucleotide. In embodiments, the nuclease-resistant nucleotide prevents cleavage of the primary TAC and the amplification primer by the site-specific nuclease. Nuclease-resistant nucleotides are further described herein. In embodiments, the nuclease-resistant nucleotide comprises a 2′-O-Methyl (2′OMe) moiety, a 2′-O-(2-Methoxyethyl) (2′MOE) moiety, a locked nucleic acid (LNA), a phosphorothioate linkage, a 2′-fluoro moiety, or a combination thereof.

In embodiments, the site-specific nuclease, e.g., Cas13 or Cas12, is an RNA-guided nuclease. In embodiments, the site-specific nuclease forms a complex with a guide polynucleotide, e.g., a guide RNA. Methods of designing and making guide RNA to form a complex with a Cas13 or Cas12 nuclease are described herein. As described herein, the guide RNA is a nucleic acid comprising a tracrRNA and a crRNA, which is complementary to a target sequence (e.g., nucleic acid of interest). In embodiments, the guide RNA is a single guide RNA (sgRNA) comprising both the tracrRNA and the crRNA. In embodiments, the guide RNA comprises the tracrRNA and the crRNA on separate polynucleotides. In embodiments, the guide polynucleotide comprises a complementary sequence to the nucleic acid of interest. In embodiments, the site-specific nuclease, e.g., Cas13 or Cas12, binds to the nucleic acid of interest via complementarity between the guide polynucleotide and the nucleic acid of interest. In embodiments, the nucleic acid of interest is a polynucleotide in a sample, wherein the entire polynucleotide binds to the site-specific nuclease, e.g., Cas13 or Cas12. In embodiments, the nucleic acid of interest is a portion or region of another compound, e.g., a longer polynucleotide, wherein a portion of the longer polynucleotide does not bind to the site-specific nuclease, e.g., Cas13 or Cas12. In embodiments, the site-specific nuclease, e.g., Cas13 or Cas12, cleaves the nucleic acid of interest following the binding. In embodiments, binding and/or cleavage of the site-specific nuclease, e.g., Cas13 or Cas12, to the nucleic acid of interest activates the collateral nuclease activity of the site-specific nuclease.

In embodiments, the oligonucleotide detection reagent comprises a single-stranded oligonucleotide. In embodiments, the oligonucleotide detection reagent is capable of being cleaved by the collateral nuclease activity of the site-specific nuclease, e.g., Cas13 or Cas12. In embodiments, the method comprises contacting the sample comprising the nucleic acid of interest with: (i) the site-specific nuclease and (ii) the oligonucleotide detection reagent, wherein the site-specific nuclease (1) binds to and/or cleaves the nucleic acid of interest as described herein and (2) collaterally cleaves the oligonucleotide detection reagent. In embodiments, the sample is simultaneously or substantially simultaneously contacted with (i) the site-specific nuclease and (ii) the oligonucleotide detection reagent. In embodiments, the sample is contacted with the site-specific nuclease prior to being contacted with the oligonucleotide detection reagent.

In embodiments, the oligonucleotide detection reagent comprises a primary TAC; a secondary TAC; and a detectable label. In embodiments, the detectable label is positioned adjacent to the primary TAC on the oligonucleotide detection reagent. In embodiments, the oligonucleotide detection reagent comprises, in 5′ to 3′ order, the secondary TAC, the primary TAC, and the detectable label. In embodiments, the oligonucleotide detection reagent comprises, in 3′ to 5′ order, the secondary TAC, the primary TAC, and the detectable label. In embodiments, collateral cleavage of the oligonucleotide detection reagent removes the secondary TAC from the oligonucleotide detection reagent, thereby generating (i) a cleaved secondary TAC and (ii) a first cleaved oligonucleotide comprising the primary TAC and the detectable label.

In embodiments, the oligonucleotide detection reagent is about 20 to about 300, about 25 to about 280, about 30 to about 250, about 35 to about 220, about 40 to about 200, about 45 to about 180, about 50 to about 150, about 55 to about 120, about 60 to about 100, or about 65 to about 80 nucleotides in length.

In embodiments, the oligonucleotide detection reagent further comprises a nuclease cleavage site. In embodiments, the nuclease cleavage site comprises a sequence at which the site-specific nuclease preferentially cleaves during collateral cleavage. In embodiments, the nuclease cleavage site comprises a poly ribouridine (rU) sequence. In embodiments, the poly rU sequence comprises at least or about 2, at least or about 3, at least or about 4, at least or about 5, at least or about 6, at least or about 7, at least or about 8, at least or about 9, or at least or about 10 rU nucleotides. In embodiments, the nuclease cleavage site comprises an RNA dinucleotide. Preferred RNA dinucleotides, e.g., for cleavage by Cas13 or Cas12 are described herein. In embodiments, the nuclease cleavage site is positioned between the secondary TAC and the primary TAC. Thus, in embodiments, the oligonucleotide detection reagent comprises, in 5′ to 3′ order, the secondary TAC, the nuclease cleavage site, the primary TAC, and the detectable label. In embodiments, the site-specific nuclease cleavages the oligonucleotide detection reagent at the nuclease cleavage site, thereby generating (i) the cleaved secondary TAC and (ii) the first cleaved oligonucleotide comprising the primary TAC and the detectable label.

In embodiments, a reaction mixture comprising the first cleaved oligonucleotide, cleaved secondary TAC, and uncleaved oligonucleotide detection reagent is formed following contacting the sample with the site-specific nuclease and the oligonucleotide detection reagent. In embodiments, the presence of uncleaved oligonucleotide detection reagent and/or second cleaved oligonucleotide interferes with one or more of the downstream steps of the method, e.g., immobilization and/or detection of the first cleaved oligonucleotide. In embodiments, the cleaved secondary TAC and/or uncleaved oligonucleotide detection reagent are separated from the first cleaved oligonucleotide. In embodiments, the separating comprises contacting the reaction mixture (comprising the first cleaved oligonucleotide, second cleaved oligonucleotide, and uncleaved oligonucleotide detection reagent) with a binding surface comprising a secondary targeting agent, wherein the cleaved secondary TAC and the uncleaved oligonucleotide detection reagent bind to the binding surface. In embodiments, the first cleaved oligonucleotide does not comprise the secondary TAC and therefore does not bind to the binding surface. In embodiments, the binding of the cleaved secondary TAC and the uncleaved oligonucleotide detection reagent to the binding surface reduces or eliminates interference with the immobilization and/or detection of the first cleaved oligonucleotide.

In embodiments, the secondary TAC is a binding partner of the secondary targeting agent on the binding surface. In embodiments, the secondary TAC and the secondary targeting gent comprise a binding pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the secondary targeting agent and secondary TAC are cross-reactive moieties, e.g., thiol and maleimide or iodoacetamide; aldehyde and hydrazide; or azide and alkyne or cycloalkyne. In embodiments, the secondary TAC comprises biotin, and the secondary targeting agent comprises avidin or streptavidin. In embodiments, the secondary TAC is an oligonucleotide of about 5 to about 100, about 6 to about 90, about 7 to about 80, about 8 to about 70, about 9 to about 60 nucleotides, about 10 to about 50, about 15 to about 45, about 20 to about 40, about 20 to about 30, or about 30 to about 35 nucleotides in length.

In embodiments, the method comprises, following binding of the cleaved secondary TAC and the uncleaved oligonucleotide detection reagent to the binding surface, immobilizing the first cleaved oligonucleotide to a detection surface comprising a primary targeting agent.

In embodiments, the primary TAC is a binding partner of a primary targeting agent on a detection surface. In embodiments, the primary TAC and the primary targeting agent comprise a binding pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the primary targeting agent and the primary TAC are cross-reactive moieties, e.g., thiol and maleimide or iodoacetamide; aldehyde and hydrazide; or azide and alkyne or cycloalkyne. In embodiments, the primary TAC and the primary targeting agent comprise complementary oligonucleotides. In embodiments, the primary TAC and the primary targeting agent are substantially unreactive with the secondary TAC and the secondary targeting agent. In embodiments, the primary TAC and the primary targeting agent comprise complementary oligonucleotides, and the secondary TAC and the secondary targeting agent comprise biotin and avidin/streptavidin. In embodiments, the primary TAC and the primary targeting agent comprise complementary oligonucleotides, and the secondary TAC and the secondary targeting agent comprise complementary oligonucleotides that are substantially non-hybridizable to the primary TAC or primary targeting agent. In embodiments, the primary TAC and the primary targeting agent comprise complementary oligonucleotides, and the immobilizing comprises hybridizing the primary TAC of the first cleaved oligonucleotide to the primary targeting agent on the detection surface. In embodiments, the primary TAC and the primary targeting agent are at least 90%, at least 95%, at least 98%, at least 99%, or 100% complementary. In embodiments, the primary TAC is an oligonucleotide of about 5 to about 100, about 6 to about 90, about 7 to about 80, about 8 to about 70, about 9 to about 60 nucleotides, about 10 to about 50, about 15 to about 45, about 20 to about 40, about 20 to about 30, about 20 to about 35, or about 30 to about 35 nucleotides in length. In embodiments, the primary TAC comprises any of SEQ ID NOs:68-71.

In embodiments, the method comprises detecting the immobilized first cleaved oligonucleotide on the detection surface. In embodiments, the cleaved secondary TAC and the uncleaved oligonucleotide detection reagent on the binding surface are substantially undetected. As used herein, the term “substantially undetected” means that less than 20%, less than 15%, less than 10%, less than 5%, less than 2%, or less than 1% of the total detected detectable label is from a detectable label that is not on the first cleaved oligonucleotide. In embodiments, the binding surface prevents detection of the cleaved secondary TAC and the uncleaved oligonucleotide detection reagent. In embodiments, the method further comprises, prior to the detecting, separating the binding surface comprising the secondary TAC from the detection surface comprising the first cleaved oligonucleotide. In embodiments, the separating comprises placing the binding surface at a distal location from the detection surface. In embodiments, the separating comprises placing the binding surface at a distance of at least about 10 μm from the detection surface, e.g., about 10, 15, 20, 25, 30, 35, 40, 45, 50, 60, 70, 80, 90, 100, or more than 100 μm from the detection surface. For example, in embodiments where the detectable label comprises an electrochemiluminescence (ECL) label and the detecting comprises applying a voltage waveform to the detection surface, generating an ECL signal, and detecting the ECL signal, the binding surface provides sufficient separation from the detection surface such that the uncleaved oligonucleotide detection reagent on the binding surface is substantially unresponsive to the voltage waveform and therefore does not generate a detectable ECL signal. In embodiments, the term “substantially unresponsive” means that less than 20%, less than 15%, less than 10%, less than 5%, less than 2%, or less than 1% of the total generated ECL signal is from an ECL label that is not on the first cleaved oligonucleotide.

In embodiments, the detecting comprises measuring the amount of detectable label on the detection surface. In embodiments, the detectable label is detectable by light scattering, optical absorbance, fluorescence, chemiluminescence, electrochemiluminescence (ECL), bioluminescence, phosphorescence, radioactivity, magnetic field, or combinations thereof. In embodiments, the detectable label comprises phycoerythrin (PE). In embodiments, the detectable label comprises a β-galactosidase (β-gal) enzyme that can be detected by fluorescence detection when the β-gal enzyme cleaves a substrate such as resorufin-β-D-galactopyranoside to yield a fluorescent signal. In embodiments, the detectable label comprises an ECL label, and the detecting comprises measuring an ECL signal. In embodiments, the ECL label comprises ruthenium. ECL labels, ECL assays, and instrumentation for conducting ECL assays are further described herein.

In embodiments, the method comprises contacting the sample with (i) the site-specific nuclease and (ii) multiple copies of the oligonucleotide detection reagent. In embodiments, the collateral nuclease activity of the site-specific nuclease cleaves the multiple copies of the oligonucleotide detection reagent, thereby generating a plurality of first cleaved oligonucleotides.

In embodiments, the method further comprises, following contacting the sample with (i) the site-specific nuclease and (ii) a first copy of the oligonucleotide detection reagent as described herein, contacting the sample with one or more additional copies of the oligonucleotide detection reagent. In embodiments, the method further comprises contacting the sample with a second nuclease, wherein the second nuclease is activated upon cleavage of the first copy of the oligonucleotide detection reagent and cleaves the one or more additional copies of the oligonucleotide detection reagent, thereby generating a plurality of first cleaved oligonucleotides. In embodiments, the oligonucleotide detection reagent further comprises a second nuclease cleavage site. In embodiments, the second nuclease cleavage site is positioned between the primary TAC and the secondary TAC, wherein the second nuclease cleaves the one or more additional copies of the oligonucleotide detection reagent at the second nuclease cleavage site. In embodiments, the second nuclease is Csm6. Csm6 is further described herein. In embodiments, the Csm6 is Enteroccocus italicus Csm6 (EiCsm6), Lactobacillus salivarius Csm6 (LsCsm6), or Therms thermophilus Csm6 (TtCsm6). In embodiments, the second nuclease increases sensitivity of the method by cleaving additional copies of the oligonucleotide detection reagent to form a plurality of first cleaved oligonucleotides.

In embodiments, the method comprises immobilizing the plurality of first cleaved oligonucleotides to the detection surface; and detecting the plurality of immobilized first cleaved oligonucleotides. In embodiments, the plurality of first cleaved oligonucleotides amplifies the assay signal. In embodiments, the method has increased sensitivity for detecting the nucleic acid of interest as compared to a method that does not amplify the assay signal as described herein. In embodiments, the method is capable of detecting a lower amount of nucleic acid of interest in a sample as compared with a method that does not form the plurality of first cleaved oligonucleotides as described herein.

### Multiplexed Embodiments

In embodiments, the method is a multiplexed method for measuring multiple nucleic acids of interest in a sample. In embodiments, each nucleic acid of interest comprises a unique sequence. In embodiments, the multiplexed method detects multiple nucleic acids of interest simultaneously or substantially simultaneously.

In embodiments, the multiplexed method comprises: (a) contacting the sample with a plurality of site-specific nucleases and a plurality of oligonucleotide detection reagents, wherein each oligonucleotide detection reagent comprises a primary TAC, a secondary TAC, and a detectable label, wherein, for each unique nucleic acid of interest, a site-specific nuclease binds to the unique nucleic acid of interest and collaterally cleaves an oligonucleotide detection reagent comprising a unique nuclease cleavage site for the site-specific nuclease to generate (1) a cleaved secondary TAC and (2) a first cleaved oligonucleotide comprising a unique primary TAC, thereby generating (i) a plurality of secondary TACs and (ii) a plurality of first cleaved oligonucleotides, wherein each first cleaved oligonucleotide comprises a unique primary TAC; (b) binding the plurality of secondary TACs, uncleaved oligonucleotide detection reagent, or both, to a binding surface comprising a plurality of secondary targeting agents; (c) immobilizing the plurality of first cleaved oligonucleotides to a detection surface comprising a plurality of primary targeting agents, wherein each primary targeting agent is a binding partner of a unique primary TAC; and (d) detecting the plurality of first cleaved oligonucleotides bound to the detection surface, wherein the plurality of secondary TACs and the uncleaved oligonucleotide detection reagent on the binding surface are substantially undetected, thereby detecting the multiple nucleic acids of interest in the sample.

In embodiments, each of the plurality of oligonucleotide detection reagents comprises a same secondary TAC. In embodiments, the secondary TAC of each oligonucleotide detection reagent comprises biotin. Thus, in embodiments, the binding surface is capable of binding to all of the cleaved secondary TACs and uncleaved oligonucleotide detection reagents. In embodiments, the binding of the cleaved secondary TACs and uncleaved oligonucleotide detection reagents to the binding surface reduces or eliminates interference with immobilization and/or detection of the plurality of first cleaved oligonucleotides, as described herein.

In embodiments, an oligonucleotide detection reagent that corresponds to a unique nucleic acid of interest comprises a unique nuclease cleavage site. Thus, the oligonucleotide detection reagent for a particular nucleic acid of interest will be cleaved only if a site-specific nuclease that recognizes and cleaves the unique nuclease cleavage site, binds to and/or cleaves that particular nucleic acid of interest in the sample. In embodiments, the site-specific nuclease is a Cas13 nuclease. Cas13 nucleases isolated from different organisms can recognize different nuclease cleavage sites, e.g., RNA dinucleotides. For example, the preferred RNA dinucleotide for LwaCas13a, CcaCas13b, LbaCas13a, and PsmCas13b are AU, UC, AC, and GA, respectively. In embodiments, the plurality of oligonucleotide detection reagents comprises first, second, third, and fourth oligonucleotide detection reagents, wherein the first oligonucleotide detection reagent comprises an AU nuclease cleavage site; the second oligonucleotide detection reagent comprises an UC nuclease cleavage site; the third oligonucleotide detection reagent comprises an AC nuclease cleavage site; and the fourth oligonucleotide detection reagent comprises an GA nuclease cleavage site. In embodiments, the plurality of site-specific nucleases comprises LwaCas13a, CcaCas13b, LbaCas13a, and PsmCas13b.

In embodiments, an oligonucleotide detection reagent that corresponds to a unique nucleic acid of interest comprises a unique primary TAC. In embodiments, each unique primary TAC comprises a unique oligonucleotide sequence that is substantially non-hybridizable to any other unique oligonucleotide sequence in the plurality of oligonucleotide detection reagents. In embodiments, the detection surface comprises multiple binding domains, wherein each binding domain comprises a unique primary targeting agent. Thus, in embodiments, the first cleaved oligonucleotide, comprising a unique primary TAC, immobilized in each binding domain corresponds to a unique nucleic acid of interest. Binding domains are further described herein.

In embodiments, the multiple nucleic acids of interest are detected by detecting the first cleaved oligonucleotide in the binding domains, wherein each binding domain corresponds to a unique nucleic acid of interest. In embodiments, each of the plurality of oligonucleotide detection reagents comprises a same detectable label, and the unique nucleic acids of interest are detected based on the first cleaved oligonucleotides in their corresponding binding domains. In embodiments, each unique oligonucleotide detection reagents comprises a unique detectable label, and the unique nucleic acids of interest are detected based on the unique detectable labels. Detectable labels and detection methods are further described herein.

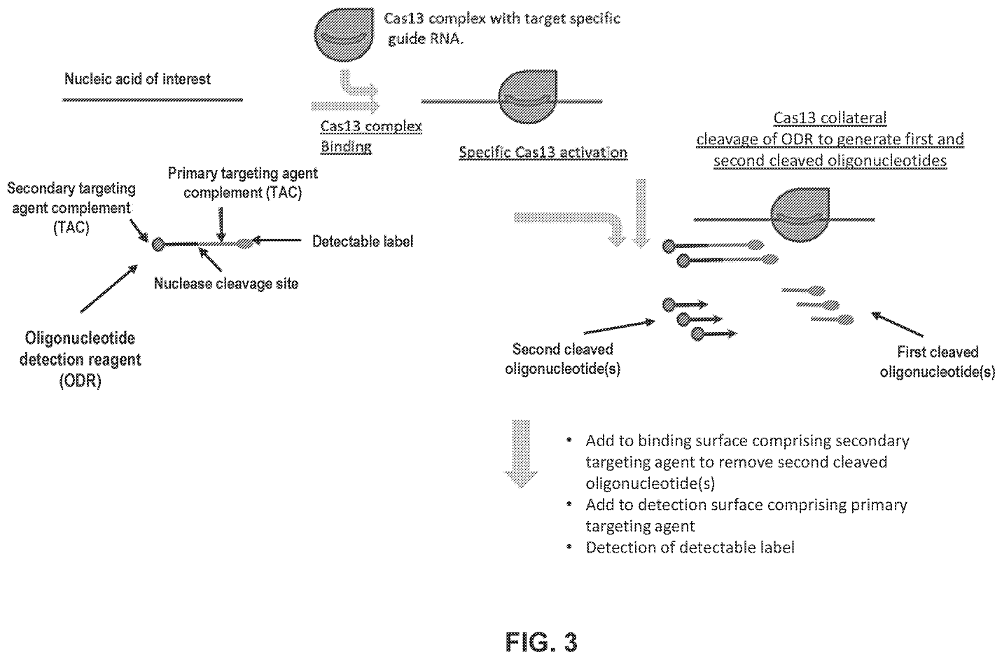

An embodiment of the method is illustrated in FIG. 3. In FIG. 3, an oligonucleotide detection reagent comprises, in 5′ to 3′ order, a secondary TAC, a nuclease cleavage site, a primary TAC, and a detectable label. A Cas13 complex binds a nucleic acid of interest, thereby activating collateral cleavage activity of the Cas13. The Cas13 collaterally cleaves one or more copies of the oligonucleotide detection reagent, thereby generating one or more of: (i) first cleaved oligonucleotides, each comprising the primary TAC and detectable label; and (ii) second cleaved oligonucleotides, each comprising the secondary TAC. The reaction mixture sample is incubated on a binding surface comprising a secondary targeting agent to bind the one or more second cleaved oligonucleotides, thereby separating the second cleaved oligonucleotide(s) from the first cleaved oligonucleotide(s). The resulting reaction mixture sample is then incubated on a detection surface comprising a primary targeting agent to immobilize the one or more first cleaved oligonucleotides onto the detection surface. In embodiments, the detectable label(s) of the immobilized first cleaved oligonucleotide(s) are detected as described herein. In embodiments, the nucleic acid of interest is detected via detection of the immobilized first cleaved oligonucleotide(s).

An exemplary protocol for performing the multiplexed method comprises:

1. Preparing the samples comprising the nucleic acids of interest. In embodiments, the preparing comprises extracting a nucleic acid (e.g., genomic DNA or RNA) from an organism of interest (e.g., a virus) that contains the nucleic acids of interest. In embodiments, the preparing further comprises producing cDNA from a genomic RNA, e.g., use reverse transcriptase. In embodiments, the preparing further comprises producing a target RNA from cDNA, e.g., using RNA polymerase.

2A. Incubating multiple sample reaction mixtures, comprising a Cas enzyme (e.g., about 10 to about 100 nM, or about 20 to about 80 nM, or about 30 to about 60 nM, about 40 to 50 nM, or about 45 nM of one or more unique Cas13 enzymes, each one corresponding to a unique nuclease cleavage RNA dinucleotide site), guide RNA targeting the nucleic acid of interest (e.g., about 5 to about 50 nM, about 10 to about 40 nM, about 20 to about 30 nM, about 22 to about 25 nM, or about 22.5 nM), the oligonucleotide detection reagents (e.g., about 0.05 to about 100 nM, about 0.1 to about 80 nM, about 0.2 nM to about 60 nM, about 0.3 nM to about 50 nM, about 0.4 nM to about 40 nM, about 0.1 to about 20 nM, or about 0.1 to about 10 nM of one or more oligonucleotide detection reagents, each one comprising a unique primary TAC), and the samples that comprise the multiple nucleic acids of interest. In embodiments, each unique primary TAC comprises a unique nucleic acid sequence. In embodiments, the sample reaction mixture is in an assay buffer of pH about 6 to about 8, about 6.5 to about 7.5, or about 6.7 to about 7. In embodiments, the sample reaction mixture comprises a reaction volume of about 10 μL to about 1 mL, about 20 μL to about 700 μL, about 50 μL to about 500 μL, about 70 μL to about 200 μL, about 90 to about 150 μL, or about 100 μL. In embodiments, the sample reaction mixture is incubated for about 10 minutes to about 6 hours, about 30 minutes to about 4 hours, or about 1 hour to about 3 hours. In embodiments, the sample reaction mixture is incubated at about 20° C. to about 50° C., about 25° C. to about 45° C., about 30° C. to about 40° C., or about 37° C. In embodiments, the sample reaction mixture is incubated for about 1 hour to about 3 hours at about 37° C.

2B. Preparing an assay plate. In embodiments, the assay plate comprises multiple binding domains in each well, wherein each binding domain comprises a unique primary targeting agent that corresponds to a unique primary TAC on the oligonucleotide detection reagent. In embodiments, each unique primary targeting agent comprises a unique nucleic acid sequence that is complementary to its corresponding primary TAC. In embodiments, the assay plate is a 96-well plate. In embodiments, the assay plate is blocked with a blocking solution. In embodiments, the blocking solution reduces and/or eliminates non-specific binding to the primary targeting agent on the assay plate. In embodiments, following the washing, a hybridization buffer is added to the assay plate (e.g., about 10 to about 50 μL, about 20 to about 40 μL, or about 30 μL per well of the assay plate). In embodiments, the hybridization buffer facilitates binding of the primary TAC to the primary targeting agent.

In embodiments, steps 1 and 2 are performed simultaneously or substantially simultaneously. In embodiments, the producing of cDNA from genomic RNA and/or the producing RNA from cDNA of step 1, and the incubating of step 2 are performed in the same reaction mixture.

3A. Incubating the sample reaction on the assay plate comprising the hybridization buffer. In embodiments, about 10 to about 100 μL, about 20 to about 80 μL, about 30 to about 70 μL, about 40 to about 60 μL, or about 50 μL of the sample reaction is added to a well of the assay plate. In embodiments, the sample reaction is incubated for about 10 minutes to about 4 hours, about 30 minutes to about 2 hours, or about 1 hour. In embodiments, the sample reaction is incubated at about 15° C. to about 40° C., about 20° C. to about 37° C., about 25° C. to about 30° C., or about 27° C. In embodiments, the sample reaction is incubated for about 1 hour at about 27° C. In embodiments, the assay plate is washed, e.g., with PBS, following the incubating.

3B. Removing second cleaved oligonucleotide and/or uncleaved oligonucleotide detection reagent. In embodiments, the removing comprises contacting the sample reaction with magnetic beads comprising a secondary targeting agent. In embodiments, the secondary targeting agent is a binding partner of a secondary TAC and/or an amplification blocker on the oligonucleotide detection reagent. In embodiments, the magnetic beads are incubated with the sample reactions for about 10 minutes to about 4 hours, about 30 minutes to about 2 hours, or about 1 hour. In embodiments, the magnetic beads are incubated with the sample reaction at about 20° C. to about 50° C., about 25° C. to about 45° C., about 30° C. to about 40° C., or about 37° C. In embodiments, the magnetic beads are incubated with the sample reaction for about 1 hour at about 37° C. In embodiments, following the incubation, the beads are removed or separated (e.g., concentrated on a side of the sample reaction container such that the beads are no longer in contact with the sample reaction) from the sample reaction by contacting the sample reaction container with a magnet.

4. Reading the plate. In embodiments, about 50 to about 500 μL, about 100 to about 300 μL, or about 150 μL of a read buffer is added to the assay plate well. In embodiments, the assay plate is read on a plate reader immediately or substantially immediately following addition of the read buffer.

### Assay Embodiment IV. Collateral Cleavage, Blocker Destabilization and Detection

In embodiments, the invention provides a method for detecting a nucleic acid of interest in a sample, comprising: (a) contacting the sample with a site-specific nuclease comprising collateral cleavage activity, and an oligonucleotide detection reagent, wherein the oligonucleotide detection reagent comprises: (i) a targeting agent complement (TAC); (ii) a targeting agent blocker that is complementary to at least a portion of the TAC; (iii) a nuclease cleavage site; and (iv) a detectable label; wherein the TAC and the targeting agent blocker are hybridized, wherein the site-specific nuclease binds to the nucleic acid of interest and collaterally cleaves the oligonucleotide detection reagent at the nuclease cleavage sequence, thereby (i) destabilizing hybridization of the TAC and the targeting agent blocker and (ii) generating an unblocked oligonucleotide comprising the TAC and the detectable label; (b) immobilizing the unblocked oligonucleotide to a detection surface comprising a targeting agent, wherein the targeting agent is a binding partner of the TAC, wherein uncleaved oligonucleotide detection reagent does not substantially bind to the detection surface; and (c) detecting the unblocked oligonucleotide bound to the detection surface, thereby detecting the nucleic acid of interest in the sample.

In embodiments, the invention provides a method for detecting a nucleic acid of interest in a sample, comprising: (a) contacting the sample with a site-specific nuclease comprising collateral cleavage activity, and an oligonucleotide detection reagent, wherein the oligonucleotide detection reagent comprises: (i) a TAC; (ii) a targeting agent blocker that is complementary to at least a portion of the TAC; (iii) a nuclease cleavage site; and (iv) an amplification primer; wherein the TAC and the targeting agent blocker are hybridized, wherein the site-specific nuclease binds to the nucleic acid of interest and collaterally cleaves the oligonucleotide detection reagent at the nuclease cleavage sequence, thereby (i) destabilizing hybridization of the TAC and the targeting agent blocker and (ii) generating an unblocked oligonucleotide comprising the TAC and the amplification primer; (b) immobilizing the unblocked oligonucleotide to a detection surface comprising a targeting agent, wherein the targeting agent is a binding partner of the TAC, wherein uncleaved oligonucleotide detection reagent does not substantially bind to the detection surface; (c) extending the unblocked oligonucleotide to form an extended oligonucleotide; and (d) detecting the extended oligonucleotide, thereby detecting the nucleic acid of interest in the sample.

In embodiments, the nucleic acid of interest is a single-stranded oligonucleotide. In embodiments, the nucleic acid of interest is DNA, e.g., single-stranded DNA (ssDNA). In embodiments, the nucleic acid of interest is RNA, e.g., single-stranded RNA (ssRNA). Exemplary nucleic acids of interest and samples are provided herein.

In embodiments, the sample comprising the nucleic acid of interest is contacted with a site-specific nuclease. In embodiments, the site-specific nuclease binds to the nucleic acid of interest. In embodiments, the site-specific nuclease is a Cas nuclease. Cas nucleases are further described herein. In embodiments, the Cas nuclease has collateral nuclease activity. Collateral nuclease activity, e.g., of Cas nucleases, is further described herein. In embodiments, the Cas nuclease is capable of collaterally cleaving a single-stranded oligonucleotide, e.g., RNA or ssDNA.

In embodiments, the nucleic acid of interest is RNA. In embodiments, oligonucleotide detection reagent is RNA. In embodiments, the site-specific nuclease is a Cas13 nuclease, as described herein. In embodiments, the Cas13 has collateral nuclease activity. In embodiments, the Cas13 is capable of collaterally cleaving RNA. In embodiments, the Cas13 is Cas13a, Cas13b, Cas13c, or Cas13d. In embodiments, the Cas13 is LwaCas13a, CcaCas13b, LbaCas13a, or PsmCas13b. In embodiments, the Cas13 is a Cas13 nuclease as described in Table 3 herein.

In embodiments, the nucleic acid of interest is ssDNA. In embodiments, the oligonucleotide detection reagent is ssDNA. In embodiments, the site-specific nuclease is a Cas12 nuclease, as described herein. In embodiments, the Cas12 has collateral nuclease activity. In embodiments, the Cas12 is capable of collaterally cleaving ssDNA. In embodiments, the Cas12 is Cas12a or Cas12b. In embodiments, the Cas12 is LbaCas12a, AsCas12, FnCas12a or AaCas12b. In embodiments, the Cas12 is a Cas12 nuclease as described in Table 2 herein.

In embodiments where the site-specific nuclease is Cas12 and the oligonucleotide detection reagent is ssDNA, the TAC and/or the amplification primer each further comprises a nuclease-resistant nucleotide. In embodiments, the nuclease-resistant nucleotide prevents cleavage of the TAC and/or the amplification primer by the site-specific nuclease. Nuclease-resistant nucleotides are further described herein. In embodiments, the nuclease-resistant nucleotide comprises a 2′-O-Methyl (2′OMe) moiety, a 2′-O-(2-Methoxyethyl) (2′MOE) moiety, a locked nucleic acid (LNA), a phosphorothioate linkage, a 2′-fluoro moiety, or a combination thereof.

In embodiments, the site-specific nuclease, e.g., Cas13 or Cas12, is an RNA-guided nuclease. In embodiments, the site-specific nuclease forms a complex with a guide polynucleotide, e.g., a guide RNA. Methods of designing and making guide RNA to form a complex with a Cas13 or Cas12 nuclease are described herein. As described herein, the guide RNA is a nucleic acid comprising a tracrRNA and a crRNA, which is complementary to a target sequence (e.g., nucleic acid of interest). In embodiments, the guide RNA is a single guide RNA (sgRNA) comprising both the tracrRNA and the crRNA. In embodiments, the guide RNA comprises the tracrRNA and the crRNA on separate polynucleotides. In embodiments, the guide polynucleotide comprises a complementary sequence to the nucleic acid of interest. In embodiments, the site-specific nuclease, e.g., Cas13 or Cas12, binds to the nucleic acid of interest via complementarity between the guide polynucleotide and the nucleic acid of interest. In embodiments, the nucleic acid of interest is a polynucleotide in a sample, wherein the entire polynucleotide binds to the site-specific nuclease, e.g., Cas13 or Cas12. In embodiments, the nucleic acid of interest is a portion or region of another compound, e.g., a longer polynucleotide, wherein a portion of the longer polynucleotide does not bind to the site-specific nuclease, e.g., Cas13 or Cas12. In embodiments, the site-specific nuclease, e.g., Cas13 or Cas12, cleaves the nucleic acid of interest following the binding. In embodiments, binding and/or cleavage of the site-specific nuclease, e.g., Cas13 or Cas12, to the nucleic acid of interest activates the collateral nuclease activity of the site-specific nuclease.

In embodiments, the oligonucleotide detection reagent comprises a single-stranded oligonucleotide. In embodiments, the oligonucleotide detection reagent is capable of being cleaved by the collateral nuclease activity of the site-specific nuclease, e.g., Cas13 or Cas12. In embodiments, the method comprises contacting the sample comprising the nucleic acid of interest with: (i) the site-specific nuclease and (ii) the oligonucleotide detection reagent, wherein the site-specific nuclease (1) binds to and/or cleaves the nucleic acid of interest as described herein and (2) collaterally cleaves the oligonucleotide detection reagent. In embodiments, the sample is simultaneously or substantially simultaneously contacted with (i) the site-specific nuclease and (ii) the oligonucleotide detection reagent. In embodiments, the sample is contacted with the site-specific nuclease prior to being contacted with the oligonucleotide detection reagent.

In embodiments, the oligonucleotide detection reagent comprises a TAC; a targeting agent blocker that is complementary to at least a portion of the TAC; a nuclease cleavage site; and a detectable label. In embodiments, the oligonucleotide detection reagent comprises a TAC; a targeting agent blocker that is complementary to at least a portion of the TAC; a nuclease cleavage site; and an amplification primer.

In embodiments, the oligonucleotide detection reagent comprises a single-stranded oligonucleotide. In embodiments, the TAC and the targeting agent blocker are on a contiguous strand of the oligonucleotide detection reagent. In embodiments, the nuclease cleavage site is positioned between the TAC and the targeting agent blocker. In embodiments, the oligonucleotide detection reagent comprises, in 5′ to 3′ order, the targeting agent blocker, the nuclease cleavage site, the TAC, and the detectable label. In embodiments, the oligonucleotide detection reagent comprises, in 3′ to 5′ order, the targeting agent blocker, the nuclease cleavage site, the TAC, and the detectable label. In embodiments, the oligonucleotide detection reagent comprises, in 5′ to 3′ order, the targeting agent blocker, the nuclease cleavage site, the TAC, and the amplification primer. In embodiments, the oligonucleotide detection reagent comprises, in 3′ to 5′ order, the targeting agent blocker, the nuclease cleavage site, the TAC, and the amplification primer. In embodiments, the nuclease cleavage site forms an oligonucleotide loop structure, thereby allowing the targeting agent blocker to hybridize to the TAC. In embodiments, the oligonucleotide loop structure is a hairpin loop. In embodiments, the TAC and the targeting agent blocker are hybridized. In embodiments, the nuclease cleavage site loop structure stabilizes the hybridization of the TAC and the targeting agent blocker.

In embodiments, the oligonucleotide detection reagent comprises a double-stranded oligonucleotide. In embodiments, the TAC is on a first strand of the oligonucleotide detection reagent, and the targeting agent blocker and the nuclease cleavage site are on a second strand of the oligonucleotide detection reagent. In embodiments, the targeting agent blocker comprises a first region and a second region, and the nuclease cleavage site is positioned between the first region and the second region of the targeting agent blocker; and wherein the first region and the second region of the targeting agent blocker hybridize to a first region and a second region of the TAC, respectively. In embodiments, the first strand of the oligonucleotide detection reagent comprises, in 5′ to 3′ order: the first region of the targeting agent blocker, the nuclease cleavage site, and the second region of the targeting agent blocker. In embodiments, the second strand of the oligonucleotide detection reagent comprises, in 3′ to 5′ order: the second region of the TAC (which is complementary to the second region of the targeting agent blocker), the first region of the TAC (which is complementary to the first region of the targeting agent blocker), and the detectable label. In embodiments, the second strand of the oligonucleotide detection reagent comprises, in 3′ to 5′ order: the second region of the TAC, the first region of the TAC, and the amplification primer. In embodiments, the first strand of the oligonucleotide detection reagent comprises, in 3′ to 5′ order: the first region of the targeting agent blocker, the nuclease cleavage site, and the second region of the targeting agent blocker. In embodiments, the second strand of the oligonucleotide detection reagent comprises, in 5′ to 3′ order: the second region of the TAC, the first region of the TAC, and the detectable label. In embodiments, the second strand of the oligonucleotide detection reagent comprises, in 3′ to 5′ order: the second region of the TAC, the first region of the TAC, and the amplification primer. In embodiments, the TAC and the targeting agent blocker are hybridized. In embodiments, the presence of the nuclease cleavage site between the first and second regions of the targeting agent blocker stabilizes the hybridization of the TAC and the targeting agent blocker.

In embodiments, the nuclease cleavage site of the oligonucleotide detection reagent comprises a sequence at which the site-specific nuclease preferentially cleaves during collateral cleavage. In embodiments, the nuclease cleavage site comprises a poly ribouridine (rU) sequence. In embodiments, the poly rU sequence comprises at least or about 2, at least or about 3, at least or about 4, at least or about 5, at least or about 6, at least or about 7, at least or about 8, at least or about 9, or at least or about 10 rU nucleotides. In embodiments, the nuclease cleavage site comprises an RNA dinucleotide. Preferred RNA dinucleotides, e.g., for cleavage by Cas13 or Cas12 are described herein. In embodiments, cleavage of the oligonucleotide detection reagent at the nuclease cleavage site destabilizes the hybridization between the TAC and the targeting agent blocker, thereby generating an unblocked oligonucleotide. In embodiments, the unblocked oligonucleotide comprises the TAC and the detectable label. In embodiments, the unblocked oligonucleotide comprises the TAC and the amplification primer.

In embodiments, the unblocked oligonucleotide is immobilized to the detection surface. In embodiments, the TAC is a binding partner of a targeting agent on a detection surface. In embodiments, hybridization of the TAC to the targeting agent blocker substantially prevents binding of the TAC to the targeting agent on the detection surface. As used herein, “substantially prevents binding” means that less than 20%, less than 15%, less than 10%, less than 5%, or less than 1% of the TAC that is hybridized to the targeting agent blocker, binds to the targeting agent on the detection surface. In embodiments, the TAC and the targeting agent comprise complementary oligonucleotides. In embodiments, the TAC and the targeting agent are at least 90%, at least 95%, at least 98%, at least 99%, or 100% complementary. In embodiments, the TAC of the unblocked oligonucleotide hybridizes to the targeting agent on the detection surface. In embodiments, uncleaved oligonucleotide detection reagent substantially does not bind to the detection surface, e.g., due to the targeting agent blocker substantially preventing binding of the TAC to the targeting agent as described herein. In embodiments, the cleaved targeting agent blocker does not bind to the detection surface.

In embodiments, the oligonucleotide detection reagent is about 20 to about 300, about 25 to about 280, about 30 to about 250, about 35 to about 220, about 40 to about 200, about 45 to about 180, about 50 to about 150, about 55 to about 120, about 60 to about 100, or about 65 to about 80 nucleotides in length. In embodiments, the TAC is an oligonucleotide of about 5 to about 100, about 6 to about 90, about 7 to about 80, about 8 to about 70, about 9 to about 60 nucleotides, about 10 to about 50, about 15 to about 45, about 20 to about 40, about 20 to about 30, about 20 to about 35, or about 30 to about 35 nucleotides in length. In embodiments, the TAC comprises any of SEQ ID NOs:68-71. In embodiments, the targeting agent blocker comprises a complementary sequence to any of SEQ ID NOs:68-71. In embodiments, the TAC and the targeting agent blocker are substantially the same length. In embodiments, the TAC and the targeting agent blocker differ in length by no more than 1, 2, 3, 4, 5, 6, 7, 8, 9, or 10 nucleotides. In embodiments, the targeting agent blocker is shorter than the TAC by about 1, 2, 3, 4, 5, 6, 7, 8, 9, or 10 nucleotides. In embodiments, the TAC is shorter than the targeting agent blocker by about 1, 2, 3, 4, 5, 6, 7, 8, 9, or 10 nucleotides. In embodiments, the difference in length between the targeting agent blocker and the TAC does not affect the targeting agent blocker's ability to substantially prevent binding of the TAC to the targeting agent on the detection surface.

In embodiments, the method comprises detecting the immobilized unblocked oligonucleotide immobilized on the detection surface. In embodiments, the components that are not bound to the detection surface, e.g., uncleaved oligonucleotide detection reagent and/or cleaved targeting agent blocker, are removed from the reaction mixture, e.g., by washing, prior to the detecting step. In embodiments, the uncleaved oligonucleotide detection reagent and the cleaved targeting agent blocker are substantially undetected.

In embodiments where the oligonucleotide detection reagent comprises a detectable label, the detecting comprises measuring the amount of detectable label on the detection surface. In embodiments, the detectable label is detectable by light scattering, optical absorbance, fluorescence, chemiluminescence, electrochemiluminescence (ECL), bioluminescence, phosphorescence, radioactivity, magnetic field, or combinations thereof. In embodiments, the detectable label comprises phycoerythrin (PE). In embodiments, the detectable label comprises a β-galactosidase (β-gal) enzyme that can be detected by fluorescence detection when the β-gal enzyme cleaves a substrate such as resorufin-β-D-galactopyranoside to yield a fluorescent signal. In embodiments, the detectable label comprises an ECL label, and the detecting comprises measuring an ECL signal. In embodiments, the ECL label comprises ruthenium. ECL labels, ECL assays, and instrumentation for conducting ECL assays are further described herein.

In embodiments where the oligonucleotide detection reagent comprises an amplification primer, the detecting comprises: extending the amplification primer on the detection surface to form an extended oligonucleotide; and detecting the extended oligonucleotide. Amplification primers and extension methods are further described herein. In embodiments, the amplification primer is about 1 to about 50, about 5 to about 45, about 10 to about 40, about 13 to about 35, about 15 to about 30, about 18 to about 40, about 20 to about 35, about 25 to about 30, or about 30 to about 35 nucleotides in length. In embodiments, the amplification primer is about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In embodiments, the detecting comprises: contacting the extended oligonucleotide with a labeled probe comprising a detectable label, wherein the labeled probe binds to the extended oligonucleotide; and measuring the amount of labeled probe bound to the extended oligonucleotide. Extended oligonucleotides and labeled probes are further described herein. In embodiments, the labeled probe comprises a detectable label. Detectable labels are further described herein.

In embodiments, the method comprises contacting the sample with (i) the site-specific nuclease and (ii) multiple copies of the oligonucleotide detection reagent. In embodiments, the collateral nuclease activity of the site-specific nuclease cleaves the multiple copies of the oligonucleotide detection reagent, thereby generating a plurality of unblocked oligonucleotides.

In embodiments, the method further comprises, following contacting the sample with (i) the site-specific nuclease and (ii) a first copy of the oligonucleotide detection reagent as described herein, contacting the sample with one or more additional copies of the oligonucleotide detection reagent. In embodiments, the method further comprises contacting the sample with a second nuclease, wherein the second nuclease is activated upon cleavage of the first copy of the oligonucleotide detection reagent and cleaves the one or more additional copies of the oligonucleotide detection reagent, thereby generating a plurality of unblocked oligonucleotides. In embodiments, the oligonucleotide detection reagent further comprises a second cleavage site. In embodiments, the second cleavage site is positioned adjacent to the nuclease cleavage site, wherein the second nuclease cleaves the one or more additional copies of the oligonucleotide detection reagent at the second cleavage site. In embodiments, the second nuclease is Csm6. Csm6 is further described herein. In embodiments, the Csm6 is EiCsm6, LsCsm6, or TtCsm6. In embodiments, the second nuclease increases sensitivity of the method by cleaving additional copies of the oligonucleotide detection reagent to form a plurality of unblocked oligonucleotides.

In embodiments where the oligonucleotide detection reagent comprises a detectable label, the method comprises immobilizing the plurality of unblocked oligonucleotides to the detection surface; and detecting the plurality of immobilized unblocked oligonucleotides. In embodiments where the oligonucleotide detection reagent comprises an amplification primer, the method comprises immobilizing the plurality of unblocked oligonucleotides to the detection surface; extending each of the immobilized plurality of unblocked oligonucleotides to form a plurality of extended oligonucleotides; and detecting the plurality of extended oligonucleotides. In embodiments, the plurality of unblocked oligonucleotides amplifies the assay signal. In embodiments, the method has increased sensitivity for detecting the nucleic acid of interest as compared to a method that does not amplify the assay signal as described herein. In embodiments, the method is capable of detecting a lower amount of nucleic acid of interest in a sample as compared with a method that does not form the plurality of unblocked oligonucleotides as described herein.

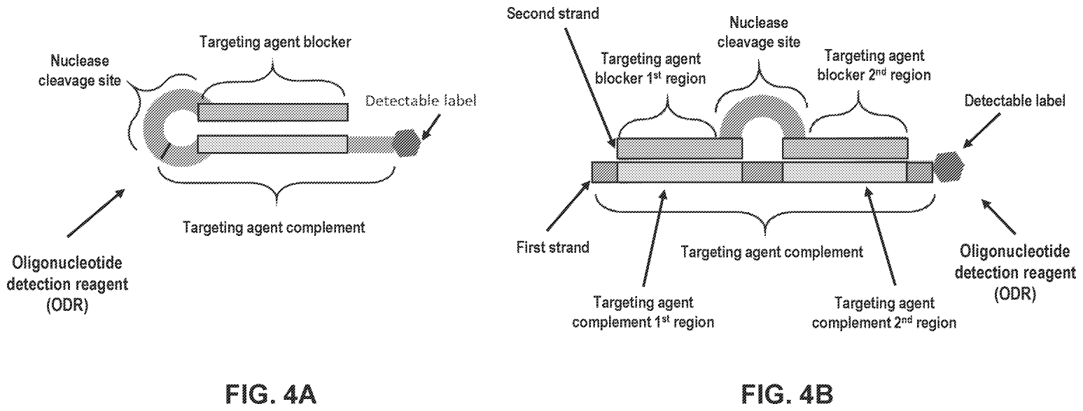

Embodiments of the oligonucleotide detection reagents described herein are illustrated in FIGS. 4A and 4B. In FIG. 4A, an oligonucleotide detection reagent comprises, in 5′ to 3′ order, a targeting agent blocker, a nuclease cleavage site, a TAC, and a detectable label. The targeting agent blocker is hybridized to the TAC. In embodiments, the nuclease cleavage site comprises a hairpin loop structure. In embodiments, a site-specific nuclease cleaves the oligonucleotide detection reagent at the nuclease cleavage site, thereby destabilizing the hybridization between the targeting agent blocker and TAC and generating an unblocked oligonucleotide comprising the TAC and the detectable label. In embodiments, the unblocked oligonucleotide is immobilized to a detection surface comprising a targeting agent that is a binding partner of the TAC. In embodiments, the detectable label of the immobilized unblocked oligonucleotide is detected as described herein.

In FIG. 4B, an oligonucleotide detection reagent comprises first and second strands, wherein a TAC is on the first strand, and a targeting agent blocker and a nuclease cleavage site are on the second strand. The targeting agent blocker comprises a first region and a second region, wherein the nuclease cleavage site is positioned between the first region and the second region of the targeting agent blocker. The first and second regions of the targeting agent blocker hybridize to first and second regions of the TAC. In embodiments, the nuclease cleavage site comprises a hairpin loop structure. In embodiments, a site-specific nuclease cleaves the oligonucleotide detection reagent at the nuclease cleavage site, thereby destabilizing the hybridization between the targeting agent blocker and TAC and generating an unblocked oligonucleotide comprising the TAC and the detectable label. In embodiments, the unblocked oligonucleotide is immobilized to a detection surface comprising a targeting agent that is a binding partner of the TAC. In embodiments, the detectable label of the immobilized unblocked oligonucleotide is detected as described herein.

### Multiplexed Embodiments

In embodiments, the method is a multiplexed method for measuring multiple nucleic acids of interest in a sample. In embodiments, each nucleic acid of interest comprises a unique sequence. In embodiments, the multiplexed method detects multiple nucleic acids of interest simultaneously or substantially simultaneously.

In embodiments, the multiplexed method comprises: (a) contacting the sample with a plurality of site-specific nucleases and a plurality of oligonucleotide detection reagents, wherein each oligonucleotide detection reagent comprises a TAC, a targeting agent blocker hybridized to the TAC, a nuclease cleavage site, and a detectable label, wherein, for each unique nucleic acid of interest, a site-specific nuclease binds to the unique nucleic acid of interest and collaterally cleaves an oligonucleotide detection reagent comprising a unique nuclease cleavage site for the site-specific nuclease to generate an unblocked oligonucleotide comprising a unique TAC; thereby generating a plurality of unblocked oligonucleotides, wherein each unblocked oligonucleotide comprises a unique TAC; (b) immobilizing the plurality of unblocked oligonucleotides to a detection surface comprising a plurality of targeting agents, wherein each targeting agent is a binding partner of a unique TAC, and wherein uncleaved oligonucleotide detection reagent does not substantially bind to the detection surface; and (c) detecting the plurality of unblocked oligonucleotides bound to the detection surface, thereby detecting the multiple nucleic acids of interest in the sample.

In embodiments, an oligonucleotide detection reagent that corresponds to a unique nucleic acid of interest comprises a unique nuclease cleavage site. Thus, the oligonucleotide detection reagent for a particular nucleic acid of interest will only be cleaved if a site-specific nuclease that recognizes and cleaves the unique nuclease cleavage site, binds to and/or cleaves that particular nucleic acid of interest in the sample. In embodiments, the site-specific nuclease is a Cas13 nuclease. As described herein, Cas13 nucleases isolated from different organisms, e.g., LwaCas13a, CcaCas13b, LbaCas13a, and PsmCas13b, can recognize different RNA dinucleotides, e.g., AU, UC, AC, and GA. In embodiments, the plurality of oligonucleotide detection reagents comprises first, second, third, and fourth oligonucleotide detection reagents, wherein the first oligonucleotide detection reagent comprises an AU nuclease cleavage site; the second oligonucleotide detection reagent comprises an UC nuclease cleavage site; the third oligonucleotide detection reagent comprises an AC nuclease cleavage site; and the fourth oligonucleotide detection reagent comprises an GA nuclease cleavage site. In embodiments, the plurality of site-specific nucleases comprises LwaCas13a, CcaCas13b, LbaCas13a, and PsmCas13b.

In embodiments, an oligonucleotide detection reagent that corresponds to a unique nucleic acid of interest comprises a unique TAC. In embodiments, each primary TAC comprises a unique oligonucleotide sequence that is substantially non-hybridizable to any other unique oligonucleotide sequence in the plurality of oligonucleotide detection reagents. In embodiments, the detection surface comprises multiple binding domains, wherein each binding domain comprises a unique targeting agent. Thus, in embodiments, the unblocked oligonucleotide, comprising a unique primary TAC, immobilized in each binding domain corresponds to a unique nucleic acid of interest. Binding domains are further described herein.

In embodiments, the multiple nucleic acids of interest are detected by detecting the unblocked oligonucleotide in the binding domains, wherein each binding domain corresponds to a unique nucleic acid of interest. In embodiments, each of the plurality of oligonucleotide detection reagents comprises a same detectable label, and the unique nucleic acids of interest are detected based on the unblocked oligonucleotides in their corresponding binding domains. In embodiments, each unique oligonucleotide detection reagents comprises a unique detectable label, and the unique nucleic acids of interest are detected based on the unique detectable labels. Detectable labels and detection methods are further described herein. In embodiments, each unique oligonucleotide detection reagent comprises a unique amplification primer that can be extended to form a unique extended oligonucleotide, and the unique nucleic acids of interest are detected based on the unique extended oligonucleotides. Extended oligonucleotides and their detection are further described herein.

An exemplary protocol for performing the multiplexed method comprises:

1A. Preparing the samples comprising the nucleic acids of interest. In embodiments, the preparing comprises extracting a nucleic acid (e.g., genomic DNA or RNA) from an organism of interest (e.g., a virus) that contains the nucleic acids of interest. In embodiments, the preparing further comprises producing cDNA from a genomic RNA, e.g., use reverse transcriptase. In embodiments, the preparing further comprises producing a target RNA from cDNA, e.g., using RNA polymerase.

1B. Preparing the oligonucleotide detection reagent. In embodiments where the TAC and the targeting agent blocker are on first and second oligonucleotide strands, the preparing comprises mixing the first strand comprising the TAC and an excess of the second strand comprising the targeting agent blocker, heating the mixture to about 90° C. to about 98° C. (e.g., about 95° C.), and cooling the mixture by about 1° C. per minute to about 20° C. to allow the first and second strands to hybridize.

2A. Incubating sample reaction mixture(s), comprising a Cas enzyme (e.g., about 10 to about 100 nM, or about 20 to about 80 nM, or about 30 to about 60 nM, about 40 to 50 nM, or about 45 nM of one or more unique Cas13 enzymes, each one corresponding to a unique nuclease cleavage RNA dinucleotide site), guide RNA targeting the nucleic acid of interest (e.g., about 5 to about 50 nM, about 10 to about 40 nM, about 20 to about 30 nM, about 22 to about 25 nM, or about 22.5 nM), the oligonucleotide detection reagents (e.g., about 0.05 to about 100 nM, about 0.1 to about 80 nM, about 0.2 nM to about 60 nM, about 0.3 nM to about 50 nM, about 0.4 nM to about 40 nM, about 0.1 to about 20 nM, or about 0.1 to about 10 nM of one or more oligonucleotide detection reagents, each one comprising a unique TAC), and the samples that comprise the multiple nucleic acids of interest. In embodiments, each unique TAC comprises a unique nucleic acid sequence. In embodiments, the sample reaction mixture is in an assay buffer of pH about 6 to about 8, about 6.5 to about 7.5, or about 6.7 to about 7. In embodiments, the sample reaction mixture comprises a reaction volume of about 10 μL to about 1 mL, about 20 μL to about 700 μL, about 50 μL to about 500 μL, about 70 μL to about 200 μL, about 90 to about 150 μL, or about 100 μL. In embodiments, the sample reaction mixture is incubated for about 10 minutes to about 6 hours, about 30 minutes to about 4 hours, or about 1 hour to about 3 hours. In embodiments, the sample reaction mixture is incubated at about 20° C. to about 50° C., about 25° C. to about 45° C., about 30° C. to about 40° C., or about 37° C. In embodiments, the sample reaction mixture is incubated for about 1 hour to about 3 hours at about 37° C.

2B. Preparing an assay plate. In embodiments, the assay plate comprises multiple binding domains in each well, wherein each binding domain comprises a unique targeting agent that corresponds to a unique TAC on the oligonucleotide detection reagent. In embodiments, each unique primary targeting agent comprises a unique nucleic acid sequence that is complementary to its corresponding primary TAC. In embodiments, the assay plate is a 96-well plate. In embodiments, the assay plate is blocked with a blocking solution. In embodiments, the blocking solution reduces and/or eliminates non-specific binding to the targeting agent on the assay plate. In embodiments, following the washing, a hybridization buffer is added to the assay plate (e.g., about 10 to about 50 μL, about 20 to about 40 μL, or about 30 μL per well of the assay plate). In embodiments, the hybridization buffer facilitates binding of the TAC to the targeting agent.

In embodiments, steps 1 and 2 are performed simultaneously or substantially simultaneously. In embodiments, the producing of cDNA from genomic RNA and/or the producing RNA from cDNA of step 1, and the incubating of step 2 are performed in the same reaction mixture.

3A. Incubating the sample reaction on the assay plate comprising the hybridization buffer. In embodiments, about 10 to about 100 μL, about 20 to about 80 μL, about 30 to about 70 μL, about 40 to about 60 μL, or about 50 μL of the sample reaction is added to a well of the assay plate. In embodiments, the sample reaction is incubated for about 10 minutes to about 4 hours, about 30 minutes to about 2 hours, or about 1 hour. In embodiments, the sample reaction is incubated at about 15° C. to about 40° C., about 20° C. to about 37° C., about 25° C. to about 30° C., or about 27° C. In embodiments, the sample reaction is incubated for about 1 hour at about 27° C. In embodiments, the assay plate is washed, e.g., with PBS, following the incubating.

3B. Removing second cleaved oligonucleotide and/or uncleaved oligonucleotide detection reagent. In embodiments, the removing comprises contacting the sample reaction with magnetic beads comprising a secondary targeting agent. In embodiments, the secondary targeting agent is a binding partner of a secondary TAC and/or an amplification blocker on the oligonucleotide detection reagent. In embodiments, the magnetic beads are incubated with the sample reactions for about 10 minutes to about 4 hours, about 30 minutes to about 2 hours, or about 1 hour. In embodiments, the magnetic beads are incubated with the sample reaction at about 20° C. to about 50° C., about 25° C. to about 45° C., about 30° C. to about 40° C., or about 37° C. In embodiments, the magnetic beads are incubated with the sample reaction for about 1 hour at about 37° C. In embodiments, following the incubation, the beads are removed or separated (e.g., concentrated on a side of the sample reaction container such that the beads are no longer in contact with the sample reaction) from the sample reaction by contacting the sample reaction container with a magnet.

4. Reading the plate. In embodiments, about 50 to about 500 ut, about 100 to about 300 μL, or about 150 μL of a read buffer is added to the assay plate well. In embodiments, the assay plate is read on a plate reader immediately or substantially immediately following addition of the read buffer.

### Assay Components

### Binding Surface

In embodiments comprising a binding surface, the binding surface comprises a secondary targeting agent immobilized thereon. In embodiments, the secondary targeting agent is indirectly immobilized on the binding surface via a binding pair, e.g., a receptor-ligand pair, an antigen-antibody pair, a hapten-antibody pair, an epitope-antibody pair, a mimotope-antibody pair, an aptamer-target molecule pair, hybridization partners, or an intercalator-target molecule pair. In embodiments, the secondary targeting agent and the binding surface comprise cross-reactive moieties, e.g., thiol and maleimide or iodoacetamide; aldehyde and hydrazide; or azide and alkyne or cycloalkyne. In embodiments, the secondary targeting agent comprises biotin, and the binding surface comprises avidin or streptavidin.

In embodiments, the binding surface comprises a planar substrate, e.g., a plate. In embodiments, the binding surface comprises a multi-well plate. In embodiments, the binding surface comprises a particle. In embodiments, the binding surface comprises a magnet. In embodiments, the binding surface comprises a paramagnetic bead. In embodiments where the binding surface comprises a particle, separating the binding surface from a reaction mixture comprises collecting the particle, e.g., via gravity filtration, centrifugation, and/or a magnetic collector, and separating the collected particles from the reaction mixture.

### Detection Surface

In embodiments, the detection surface comprises a targeting agent immobilized thereon. In embodiments, the targeting agent is directly immobilized on the detection surface. In embodiments, the targeting agent is indirectly immobilized on the detection surface via a binding pair, e.g., a receptor-ligand pair, an antigen-antibody pair, a hapten-antibody pair, an epitope-antibody pair, a mimotope-antibody pair, an aptamer-target molecule pair, hybridization partners, or an intercalator-target molecule pair. In embodiments, the targeting agent and the detection surface comprise cross-reactive moieties, e.g., thiol and maleimide or iodoacetamide; aldehyde and hydrazide; or azide and alkyne or cycloalkyne. In embodiments, the targeting agent comprises biotin, and the detection surface comprises avidin or streptavidin.

In embodiments comprising an anchoring reagent, the anchoring reagent is immobilized to the detection surface. In embodiments, the anchoring reagent is directly immobilized on the detection surface. In embodiments, the anchoring reagent is indirectly immobilized on the detection surface via a binding pair as described herein. In embodiments, the secondary binding reagents for the targeting agent and the anchoring reagent are selected such that the secondary binding reagent associated with the targeting agent are substantially non-cross-reactive with the secondary binding reagent associated with the anchoring reagent. In embodiments, the same secondary binding reagent is associated with the targeting agent and the anchoring reagent.

In embodiments, the detection surface comprises a particle. In embodiments, the detection surface comprises a paramagnetic bead. In embodiments, the detection surface comprises a well of multi-well plate. In embodiments, the detection surface comprises a cartridge. In embodiments, the detection surface comprises a plurality of distinct binding domains, and the targeting agent and anchoring reagent are located on two distinct binding domains on the surface. In embodiments, the detection surface comprises a plurality of distinct binding domains, and the targeting agent and anchoring reagent are located on the same binding domain on the detection surface. In embodiments, each distinct binding domain is positioned about 10 μm to about 100 μm apart from an adjacent distinct binding domain on the detection surface. In embodiments, each distinct binding domain is positioned less than 100 μm apart from an adjacent distinct binding domain on the detection surface. In embodiments, each distinct binding domain is positioned less than 50 μm apart from an adjacent distinct binding domain on the detection surface. In embodiments, each distinct binding domain is positioned less than 10 μm apart from an adjacent distinct binding domain on the detection surface.

In embodiments, the detection surface comprises an electrode. In embodiments, the electrode is a carbon ink electrode. In embodiments, the detecting (e.g., of a detectable label described herein) comprises applying a voltage waveform (e.g., a potential) to the electrode to general an ECL signal. In embodiments, the detection surface comprises a particle, and the method comprises collecting the particle on an electrode and applying a voltage waveform (e.g., a potential) to the electrode to generate an ECL signal.

In embodiments where the method is a multiplexed method for detecting multiple nucleic acids of interest, the detection surface comprises a plurality of binding domains, and each unique nucleic acid of interest is detected in a different binding domain. In embodiments, the detection surface comprises a multi-well plate, and each binding domain is in a different well. In embodiments, the detection surface comprises a well of a multi-well plate, and each binding domain is in a separate portion of the well. In embodiments, the plurality of binding domains is on one or more detection surfaces. In embodiments, the detection surface comprises a particle, and each binding domain is on a different particle. In embodiments, the particles are arranged in a particle array. In embodiments, the particles are coded to allow for identification of specific particles and distinguish between each binding domain.

### Analytes and Samples

In embodiments, the sample is a biological sample. In embodiments, the sample is an environmental sample. In embodiments, the sample is obtained from a human subject. In embodiments, the sample is obtained from an animal subject. In embodiments, the sample comprises a mammalian fluid, secretion, or excretion. In embodiments, the sample is a purified mammalian fluid, secretion, or excretion. In embodiments, the mammalian fluid, secretion, or excretion is whole blood, plasma, serum, sputum, lachrymal fluid, lymphatic fluid, synovial fluid, pleural effusion, urine, sweat, cerebrospinal fluid, ascites, milk, stool, a respiratory sample, bronchial/bronchoalveolar lavage, saliva, mucus, oropharyngeal swab, sputum, endotracheal aspirate, pharyngeal/nasal swab, throat swab, amniotic fluid, nasal secretions, nasopharyngeal wash or aspirate, nasal mid-turbinate swab, vaginal secretions, a surface biopsy, sperm, semen/seminal fluid, wound secretions and excretions, ear secretions or discharge, or an extraction, purification therefrom, or dilution thereof. Further exemplary biological samples include but are not limited to physiological samples, samples containing suspensions of cells such as mucosal swabs, tissue aspirates, endotracheal aspirates, tissue homogenates, cell cultures, and cell culture supernatants. In embodiments, the biological sample is a respiratory sample obtained from the respiratory tract of a subject. Examples of respiratory samples include, but are not limited to, bronchial/bronchoalveolar lavage, saliva, mucus, endotracheal aspirate, sputum, nasopharyngeal/nasal swab, throat swab, oropharyngeal swab and the like. In embodiments, the biological sample is whole blood, serum, plasma, cerebrospinal fluid (CSF), urine, saliva, sputum, endotracheal aspirate, nasopharyngeal/nasal swab, bronchoalveolar lavage, or an extraction or purification therefrom, or dilution thereof. In embodiments, the biological sample is serum or plasma. In embodiments, the plasma is in EDTA, heparin, or citrate. In embodiments, the biological sample is saliva. In embodiments, the biological sample is endotracheal aspirate. In embodiments, the biological sample is a nasal swab.

In embodiments, the sample is an environmental sample. In embodiments, the environmental sample is aqueous, including but not limited to, fresh water, drinking water, marine water, reclaimed water, treated water, desalinated water, sewage, wastewater, surface water, ground water, runoff, aquifers, lakes, rivers, streams, oceans, and other natural or non-natural bodies of water. In embodiments, the aqueous sample contains bodily solids or fluids (e.g., feces or urine) from a human subject.

Samples may be obtained from a single source described herein, or may contain a mixture from two or more sources.

In embodiments, the sample comprises or is suspected to comprise a nucleic acid of interest. In embodiments, the nucleic acid of interest is a polynucleotide in a sample, wherein the entire polynucleotide hybridizes to an oligonucleotide binding reagent in a binding complex or a site-specific nuclease as described herein. In embodiments, the nucleic acid of interest is a portion or region of another compound, e.g., a longer polynucleotide, wherein a portion of the longer polynucleotide does not hybridize to an oligonucleotide binding reagent in a binding complex or a site-specific nuclease as described herein. In embodiments, the nucleic acid of interest is double-stranded. For double-stranded nucleic acids, the sequence of interest can be present in either strand. In embodiments, the nucleic acid of interest is single-stranded. In embodiments, the nucleic acid of interest is DNA, e.g., genomic DNA, mitochondrial DNA, cDNA, whole genome amplified DNA, or a combination thereof. In embodiments, the nucleic acid of interest is RNA, e.g., ribosomal RNA, mRNA, miRNA, siRNA, RNAi, viral RNA, or a combination thereof. In embodiments, the nucleic acid of interest comprises a synthetic nucleic acid such as, e.g., a PCR product, a plasmid, a cosmid, a DNA library, a yeast artificial chromosome (YAC), a bacterial artificial chromosome (BAC), a synthetic oligonucleotide, a restriction fragment, a DNA/RNA hybrid, a PNA (peptide nucleic acid), a DNA/RNA mosaic nucleic acid, or a combination thereof. In embodiments, the nucleic acid of interest comprises a therapeutic oligonucleotide. A “therapeutic oligonucleotide” as used herein refers to an oligonucleotide capable of interacting with a biomolecule to provide a therapeutic effect. In embodiments, the therapeutic oligonucleotide is an antisense oligonucleotide (ASO).

In embodiments, the sample comprises a viral nucleic acid, e.g., viral DNA or viral RNA. In embodiments, the virus is a human pathogen virus. Pathogenic viruses are typically in the families of Adenoviridae, Picornaviridae, Herpesviridae, Hepadnaviridae, Coronaviridae, Flaviviridae, Retroviridae, Orthomyxoviridae, Paramyxoviridae, Papovaviridae, Polyomavirus, Poxviridae, Rhabdoviridae, and Togaviridae. Non-limiting examples of human pathogen viruses include smallpox virus, mumps virus, measles virus, rubella virus, chickenpox virus, Ebola virus, Zika virus, and respiratory viruses including influenza and coronaviruses. In embodiments, the virus is a respiratory virus, e.g., influenza A (FluA), influenza B (FluB), respiratory syncytial virus (RSV), a coronavirus, or a combination thereof.

In embodiments, the nucleic acid of interest comprises about 5 to about 100, about 10 to about 90, about 20 to about 80, about 30 to about 70, or about 40 to about 60 nucleotides in length. In embodiments, the nucleic acid of interest comprises about 5, 10, 15, 20, 25, 30, 35, 40, 45, 50, 55, 60, 65, 70, 75, 80, 85, 90, 95, or 100 nucleotides in length.

In embodiments, the detection limit of the method is about 1 to about 10⁶ fg/mL, about 1 to about 10⁵ fg/mL, about 1 to about 10⁴ fg/mL, about 1 to about 1000 fg/mL, or about 1 to about 100 fg/mL of the nucleic acid of interest in the sample.

### Assay Devices, Manual and Automated Embodiments

The methods herein can be conducted in a single assay chamber, such as a single well of an assay plate. The methods herein can also be conducted in an assay chamber of an assay cartridge. The assay modules, e.g., assay plates or assay cartridges, methods and apparatuses for conducting assay measurements suitable for the present invention, are described, e.g., in U.S. Pat. Nos. 8,343,526; 9,731,297; 9,921,166; 10,184,884; 10,281,678; 10,272,436; US 2004/0022677; US 2004/0189311; US 2005/0052646; US 2005/0142033; US 2018/0074082; and US 2019/0391170.

The methods herein can be performed manually, using automated technology, or both. Automated technology may be partially automated, e.g., one or more modular instruments, or a fully integrated, automated instrument. Exemplary automated systems and apparatuses are described in WO 2018/017156, WO 2017/015636, and WO 2016/164477.

In embodiments, automated systems, e.g., modular and fully integrated systems, for performing the methods herein comprises one or more of the following automated subsystems: a computer subsystem comprising hardware (e.g., personal computer, laptop, hardware processor, disc, keyboard, display, printer), software (e.g., processes such as drivers, driver controllers, and data analyzers), and/or a database; a liquid handling subsystem for sample and/or reagent handling, e.g., comprising a robotic pipetting hand, syringe, stirring apparatus, ultrasonic mixing apparatus, and/or magnetic mixing apparatus; a sample, reagent, and/or consumable storing and handling subsystem, e.g., comprising a robotic manipulator, tube or lid or foil piercing apparatus, lid removing apparatus, conveying apparatus such as linear or circular conveyor, tube rack, plate carrier, trough carrier, pipet tip carrier, plate shaker, and/or centrifuge; an assay reaction subsystem, e.g., that is fluid-based and/or consumable-based (such as tube and multi-well plate); a container and consumable washing subsystem, e.g., comprising a plate washing apparatus; a magnetic separator or magnetic particle concentrator subsystem, e.g., that is flow cell type, tube type, and/or plate type; a cell and particle detection, classification, and/or separation subsystem, e.g., comprising a flow cytometer and/or a Coulter counter; a detection subsystem, e.g., comprising a colorimetric detector, a nephelometric detector, a fluorescence detector, and/or an ECL detector; a temperature control subsystem, e.g., comprising an air handling system, air cooling system, air warming system, fan, blower, and/or water bath; a waste subsystem, e.g., comprising liquid and/or solid waste containers; a global unique identifier (GUI) detecting subsystem, e.g., comprising 1D and/or 2D barcode scanners such as flat bed and wand type scanners. In embodiments, the automated system further comprises a modular or fully integrated analytical subsystem, e.g., a chromatography system such as high-performance liquid chromatography (HPLC) or fast-protein liquid chromatography (FPLC), or a mass spectrometer.

In embodiments, systems or modules that perform sample identification and preparation are combined with, adjoined to, adjacent to, and/or robotically linked or coupled to the systems or modules that perform and/or detect the assays herein. Multiple modular systems of the same type can be combined to increase throughput. In embodiments, a modular system is combined with a module that performs other types of analysis, such as chemical, biochemical, and/or nucleic acid analysis.

In embodiments, the automated system allows batch, continuous, random-access, and/or point-of-care workflows, and single, medium, and high sample throughput.

In embodiments, the automated system comprises one or more of the following devices: a plate sealer (e.g., ZYMARK™), a plate washer (e.g., BIOTEK™, TECAN™), a reagent dispenser, automated pipetting station, and/or liquid handling station (e.g., TECAN™, ZYMARK™, LABSYSTEMS™, BECKMAN™, HAMILTON™), an incubator (e.g., ZYMARK™), a plate shaker (e.g., Q.INSTRUMENTS™, INHECO™, THERMOFISHER™), a compound library module, a sample storage module, and/or a compound and/or sample retrieval module. In embodiments, one or more of these devices is coupled to the automated system via a robotic assembly such that the entire assay process can be performed automatically. In embodiments, a container (e.g., a plate) is manually moved between the apparatus and various devices described herein (e.g., a stack of plates).

In embodiments, the automated system is configured to perform one or more of the following functions: moving consumables such as plates into, within, and out of the detection subsystem; moving consumables between other subsystems; storing the consumables; sample and reagent handling (e.g., adapted to mix reagents and/or introduce reagents into consumables); consumable shaking (e.g., for mixing reagents and/or for increasing reaction rates); consumable washing (e.g., washing plates and/or performing assay wash steps (e.g., well aspirating)); measuring a detectable signal, e.g., ECL signal, in a flow cell or a consumable such as a tube or a plate. The automated system may be configured to handle individual tubes placed in racks and/or multi-well plates such as 96 or 384 well plates.

Methods for integrating components and modules in automated systems as described herein are discussed, e.g., by Sargeant et al., “Platform Perfection,” Medical Product Outsourcing, May 17, 2010.

In embodiments, the automated system is fully automated, modular, computerized, performs in vitro quantitative and qualitative tests on a wide range of analytes, and/or performs photometric assays, ion-selective electrode measurements, and/or electrochemiluminescence (ECL) assays. In embodiments, the system comprises one or more of the following hardware units: a control unit, a core unit and at least one analytical module.

In embodiments, the control unit utilizes a graphical user interface to control all instrument functions and comprises a readout device, such as a monitor; an input device, such as keyboard and mouse; and a personal computer, e.g., using a Windows operating system. In embodiments, the core unit comprises one or more components that manage conveyance of samples to each assigned analytical module. The actual composition of the core unit depends on the configuration of the analytical modules, which can be configured by one of skill in the art using methods known in the art. In embodiments, the core unit comprises at least the sampling unit and one rack rotor as main components. In embodiments, the control unit further comprises an extension unit, e.g., a conveyor line and/or a second rack rotor. In embodiments, the core unit further comprises a sample rack loader/unloader, a port, a barcode reader (for racks and samples), a water supply, and a system interface port. In embodiments, the automated system conducts ECL assays and comprises a reagent area, a measurement area, a consumables area, and a pre-clean area.

### Composition and Kit, Embodiment I

In embodiments, the invention provides a kit for detecting a nucleic acid of interest, comprising, in one or more containers, vials, or compartments: (a) an oligonucleotide binding reagent that comprises (i) a targeting agent complement (TAC); (ii) an amplification primer; and (iii) an amplification blocker; and (b) a site-specific nuclease that forms a complex with the oligonucleotide binding reagent. In embodiments, the kit further comprises (c) a detection surface comprising a targeting agent.

Oligonucleotide binding reagents are further described herein. In embodiments, the oligonucleotide binding reagent comprises a single-stranded oligonucleotide. In embodiments, the oligonucleotide binding reagent comprises, in 5′ to 3′ order, the TAC, the amplification primer, and the amplification blocker. In embodiments, the oligonucleotide binding reagent is about 20 to about 300, about 25 to about 280, about 30 to about 250, about 35 to about 220, about 40 to about 200, about 45 to about 180, about 50 to about 150, about 55 to about 120, about 60 to about 100, or about 65 to about 80 nucleotides in length.

In embodiments, the oligonucleotide binding reagent further comprises a hybridization region comprising a complementary sequence to the nucleic acid of interest. In embodiments, the hybridization region is positioned between the amplification primer and the amplification blocker. In embodiments, the hybridization region is about 10 to about 50, about 11 to about 45, about 12 to about 40, about 13 to about 35, about 14 to about 30, about 15 to about 25, about 16 to about 24, about 17 to about 23, or about 18 to about 22 nucleotides in length. In embodiments, the hybridization region is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In embodiments, the oligonucleotide binding reagent further comprises an insertion site for inserting a complementary sequence to the nucleic acid of interest into the oligonucleotide binding reagent. In embodiments, the insertion site is positioned between the amplification primer and the amplification blocker.

In embodiments, the TAC is a binding partner of a targeting agent on a detection surface. In embodiments, the targeting agent and the detection surface are provided separately in the kit, and the kit further comprises a reagent for immobilizing the targeting agent onto the detection surface. In embodiments, the TAC and the targeting agent comprise a binding pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the targeting agent and TAC are cross-reactive moieties, e.g., thiol and maleimide or iodoacetamide; aldehyde and hydrazide; or azide and alkyne or cycloalkyne. In embodiments, the TAC and the targeting agent comprise complementary oligonucleotides. In embodiments, the TAC and the targeting agent are at least 90%, at least 95%, at least 98%, at least 99%, or 100% complementary. In embodiments, the TAC is an oligonucleotide of about 5 to about 100, about 6 to about 90, about 7 to about 80, about 8 to about 70, about 9 to about 60 nucleotides, about 10 to about 50, about 15 to about 45, about 20 to about 40, about 20 to about 30, about 20 to about 35, or about 30 to about 35 nucleotides in length. In embodiments, the TAC comprises any of SEQ ID NOs:68-71. Targeting agents and TACs are further described herein.

In embodiments, the amplification primer comprises a primer for polymerase chain reaction (PCR), ligase chain reaction (LCR), strand displacement amplification (SDA), self-sustained synthetic reaction (3SR), or an isothermal amplification method. In embodiments, the amplification primer comprises a primer for an isothermal amplification method. In embodiments, the isothermal amplification method is helicase-dependent amplification. In embodiments, the isothermal amplification method is rolling circle amplification (RCA). Amplification primers and methods are further described herein. In embodiments, the amplification primer is about 10 to about 50, about 11 to about 45, about 12 to about 40, about 13 to about 35, about 14 to about 30, about 15 to about 40, about 20 to about 35, about 25 to about 30, or about 30 to about 35 nucleotides in length. In embodiments, the amplification primer is about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In embodiments, the amplification primer comprises the sequence GACAGAACTAGACAC (SEQ ID NO:64).

In embodiments, the amplification blocker blocks amplification of the amplification primer. In embodiments, the amplification blocker comprises an oligonucleotide that blocks amplification of the amplification primer by preventing polymerase binding, inhibiting polymerase activity, and/or promoting polymer dissociation from the amplification primer. In embodiments, the amplification blocker comprises a nucleotide modification. Non-limiting examples of nucleotide modifications that block amplification include 3′-spacer C3, 3′-phosphate, 3′-dideoxy cytidine (3′-ddC), and 3′-inverted end. In embodiments, the amplification blocker comprises a PNA and/or an LNA. In embodiments, the amplification blocker comprises a 2′-O-methyl uridine, a 3′-inverted dT, a digoxigenin, a biotin, or a combination thereof. In embodiments, the amplification blocker comprises a secondary structure, e.g., a stem loop or a pseudoknot. Amplification blockers are further described herein.

In embodiments, the site-specific nuclease is a nickase. Nickases are further described herein. In embodiments, the site-specific nickase is a Cas9 nickase or a Cas12a nickase. In embodiments, the composition and/or kit further comprises a guide polynucleotide, e.g., a guide RNA. Guide polynucleotides are further described herein. In embodiments, the guide RNA comprises one or both of a tracrRNA and a crRNA. In embodiments, the guide polynucleotide comprises a complementary sequence to the nuclease binding site of the oligonucleotide binding reagent, or a complementary sequence to the nucleic acid of interest. In embodiments, the guide polynucleotide is capable of forming with a complex with the site-specific nuclease. In embodiments, the site-specific nuclease in the composition and/or the kit is complexed with the guide polynucleotide.

In embodiments, the oligonucleotide binding reagent further comprises a nuclease binding site. In embodiments, the site-specific nuclease is capable of cleaving the oligonucleotide binding reagent at the nuclease binding site. In embodiments, the nuclease binding site is positioned between the hybridization region and the amplification blocker. In embodiments, the nuclease binding site comprises at least a portion of the hybridization region, at least a portion of the amplification blocker, or both. Nuclease binding sites are further described herein.

In embodiments, the oligonucleotide binding reagent further comprises a secondary targeting agent complement (secondary TAC). In embodiments, the kit further comprises a binding surface comprising a secondary targeting agent. In embodiments, the kit further comprises a binding surface, a secondary targeting agent, and a reagent for immobilizing the secondary targeting agent onto the binding surface. In embodiments, the amplification blocker is a binding partner of the secondary targeting agent on the binding surface. In embodiments, the secondary TAC is a binding partner of the secondary targeting agent. In embodiments, the secondary TAC and the secondary targeting gent comprise a binding pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the secondary targeting agent and secondary TAC are cross-reactive moieties, e.g., thiol and maleimide or iodoacetamide; aldehyde and hydrazide; or azide and alkyne or cycloalkyne. In embodiments, the secondary TAC comprises biotin, and the secondary targeting agent comprises avidin or streptavidin. In embodiments, the secondary TAC is an oligonucleotide of about 5 to about 100, about 6 to about 90, about 7 to about 80, about 8 to about 70, about 9 to about 60 nucleotides, about 10 to about 50, about 15 to about 45, about 20 to about 40, about 20 to about 30, or about 30 to about 35 nucleotides in length. In embodiments, the TAC and the targeting agent are substantially unreactive with the secondary TAC and the secondary targeting agent. In embodiments, the TAC and the secondary TAC are on separate ends of the oligonucleotide binding reagent. In embodiments, the oligonucleotide binding reagent comprises, in 5′ to 3′ order: the TAC, the amplification primer, the hybridization region, the amplification blocker, and the secondary TAC. In embodiments, the secondary TAC is positioned adjacent to the amplification blocker on the oligonucleotide binding reagent. Secondary TACs are further described herein.

In embodiments, the detection surface comprises an anchoring reagent. In embodiments, the kit provides an anchoring reagent and a reagent for immobilizing the anchoring reagent onto the detection surface. In embodiments, the anchoring reagent comprises the sequence AAGAGAGTAGTACAGCAGCCGTCAA (SEQ ID NO:66). Methods of immobilizing anchoring reagents onto a detection surface are provided herein. In embodiments, the detection surface comprises a plurality of distinct binding domains, and the targeting agent and anchoring reagent are located on two distinct binding domains on the surface. In embodiments, the detection surface comprises a plurality of distinct binding domains, and the targeting agent and anchoring reagent are located on the same binding domain on the detection surface. In embodiments, each distinct binding domain is positioned about 10 μm to about 100 μm apart from an adjacent distinct binding domain on the detection surface. In embodiments, each distinct binding domain is positioned less than 100 μm, less than 50 μm apart, or less than 10 μm apart from an adjacent distinct binding domain on the detection surface. Detection surfaces are further described herein.

### Composition and Kit, Embodiment II

In embodiments, the invention provides an oligonucleotide detection reagent comprising: (i) a targeting agent complement (TAC); (ii) an amplification primer; and (iii) an amplification blocker. In embodiments, the invention further provides a composition comprising the oligonucleotide detection reagent, a site-specific nuclease, and a nucleic acid of interest.

In embodiments, the invention provides a kit for detecting a nucleic acid of interest, comprising, in one or more containers, vials, or compartments: (a) an oligonucleotide detection reagent that comprises (i) a targeting agent complement (TAC); (ii) an amplification primer; and (iii) an amplification blocker; and (b) a site-specific nuclease having collateral activity. In embodiments, the kit further comprises (c) a detection surface comprising a targeting agent.

Oligonucleotide detection reagents are further described herein. In embodiments, the oligonucleotide detection reagent comprises a single-stranded oligonucleotide. In embodiments, the oligonucleotide detection reagent comprises RNA. In embodiments, the oligonucleotide detection reagent comprises ssDNA. In embodiments, the oligonucleotide detection reagent comprises, in 5′ to 3′ order, the TAC, the amplification primer, and the amplification blocker. In embodiments, the oligonucleotide detection reagent comprises, in 3′ to 5′ order, the TAC, the amplification primer, and the amplification blocker. In embodiments, the oligonucleotide detection reagent is about 20 to about 300, about 25 to about 280, about 30 to about 250, about 35 to about 220, about 40 to about 200, about 45 to about 180, about 50 to about 150, about 55 to about 120, about 60 to about 100, or about 65 to about 80 nucleotides in length.

In embodiments, the TAC is a binding partner of a targeting agent on a detection surface. In embodiments, the targeting agent and the detection surface are provided separately in the kit, and the kit further comprises a reagent for immobilizing the targeting agent onto the detection surface. In embodiments, the TAC and the targeting agent comprise a binding pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the targeting agent and TAC are cross-reactive moieties, e.g., thiol and maleimide or iodoacetamide; aldehyde and hydrazide; or azide and alkyne or cycloalkyne. In embodiments, the TAC and the targeting agent comprise complementary oligonucleotides. In embodiments, the TAC and the targeting agent are at least 90%, at least 95%, at least 98%, at least 99%, or 100% complementary. In embodiments, the TAC is an oligonucleotide of about 5 to about 100, about 6 to about 90, about 7 to about 80, about 8 to about 70, about 9 to about 60 nucleotides, about 10 to about 50, about 15 to about 45, about 20 to about 40, about 20 to about 30, about 20 to about 35, or about 30 to about 35 nucleotides in length. In embodiments, the TAC comprises any of SEQ ID NOs:68-71. Targeting agents and TACs are further described herein.

In embodiments, the amplification primer comprises a primer for polymerase chain reaction (PCR), ligase chain reaction (LCR), strand displacement amplification (SDA), self-sustained synthetic reaction (3SR), or an isothermal amplification method. In embodiments, the amplification primer comprises a primer for an isothermal amplification method. In embodiments, the isothermal amplification method is helicase-dependent amplification. In embodiments, the isothermal amplification method is rolling circle amplification (RCA). In embodiments, the amplification primer is about 10 to about 50, about 11 to about 45, about 12 to about 40, about 13 to about 35, about 14 to about 30, about 15 to about 40, about 20 to about 35, about 25 to about 30, or about 30 to about 35 nucleotides in length. In embodiments, the amplification primer is about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In embodiments, the amplification primer comprises the sequence GACAGAACTAGACAC (SEQ ID NO:64). Amplification primers and methods are further described herein.

In embodiments, the amplification blocker blocks amplification of the amplification primer. In embodiments, the amplification blocker comprises an oligonucleotide that blocks amplification of the amplification primer by preventing polymerase binding, inhibiting polymerase activity, and/or promoting polymer dissociation from the amplification primer. In embodiments, the amplification blocker comprises a nucleotide modification. Non-limiting examples of nucleotide modifications that block amplification include 3′-spacer C3, 3′-phosphate, 3′-dideoxy cytidine (3′-ddC), and 3′-inverted end. In embodiments, the amplification blocker comprises a PNA and/or an LNA. In embodiments, the amplification blocker comprises a 2′-O-methyl uridine, a 3′-inverted dT, a digoxigenin, a biotin, or a combination thereof. In embodiments, the amplification blocker comprises a secondary structure, e.g., a stem loop or a pseudoknot.

In embodiments, the site-specific nuclease is a Cas nuclease. Cas nucleases having collateral activity are further described herein. In embodiments, the Cas nuclease is a Cas12 or Cas13 nuclease. In embodiments, the composition and/or kit further comprises a guide polynucleotide, e.g., a guide RNA. Guide polynucleotides are further described herein. In embodiments, the guide RNA comprises one or both of a tracrRNA and a crRNA. In embodiments, the guide polynucleotide comprises a complementary sequence to the nucleic acid of interest. In embodiments, the guide polynucleotide is capable of forming with a complex with the site-specific nuclease. In embodiments, the site-specific nuclease in the composition and/or the kit is complexed with the guide polynucleotide.

In embodiments, the oligonucleotide detection reagent further comprises a nuclease cleavage site. In embodiments, the nuclease cleavage site comprises a sequence at which the site-specific nuclease preferentially cleaves during collateral cleavage. In embodiments, the nuclease cleavage site comprises a poly ribouridine (rU) sequence. In embodiments, the poly rU sequence comprises at least or about 2, at least or about 3, at least or about 4, at least or about 5, at least or about 6, at least or about 7, at least or about 8, at least or about 9, or at least or about 10 rU nucleotides. In embodiments, the nuclease cleavage site comprises an RNA dinucleotide. In embodiments, the nuclease cleavage site is positioned between the amplification primer and the amplification blocker. Nuclease cleavage sites are further described herein.

In embodiments where the site-specific nuclease is Cas12 and the oligonucleotide detection reagent is ssDNA, each of the TAC and the amplification primer further comprises a nuclease-resistant nucleotide. Nuclease-resistant nucleotides are further described herein. In embodiments, the nuclease-resistant nucleotide comprises a 2′-O-Methyl (2′OMe) moiety, a 2′-O-(2-Methoxyethyl) (2′MOE) moiety, a locked nucleic acid (LNA), a phosphorothioate linkage, a 2′-fluoro moiety, or a combination thereof.

In embodiments, the oligonucleotide detection reagent further comprises a secondary targeting agent complement (secondary TAC). In embodiments, the kit further comprises a binding surface comprising a secondary targeting agent. In embodiments, the kit further comprises a binding surface, a secondary targeting agent, and a reagent for immobilizing the secondary targeting agent onto the binding surface. In embodiments, the amplification blocker is a binding partner of the secondary targeting agent on the binding surface. In embodiments, the secondary TAC is a binding partner of the secondary targeting agent. In embodiments, the secondary TAC and the secondary targeting gent comprise a binding pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the secondary targeting agent and secondary TAC are cross-reactive moieties, e.g., thiol and maleimide or iodoacetamide; aldehyde and hydrazide; or azide and alkyne or cycloalkyne. In embodiments, the secondary TAC is an oligonucleotide of about 5 to about 100, about 6 to about 90, about 7 to about 80, about 8 to about 70, about 9 to about 60 nucleotides, about 10 to about 50, about 15 to about 45, about 20 to about 40, about 20 to about 30, or about 30 to about 35 nucleotides in length. In embodiments, the TAC and the targeting agent are substantially unreactive with the secondary TAC and the secondary targeting agent. In embodiments, the TAC and the secondary TAC are on separate ends of the oligonucleotide detection reagent. In embodiments, the oligonucleotide detection reagent comprises, in 5′ to 3′ order: the TAC, the amplification primer, the hybridization region, the amplification blocker, and the secondary TAC. In embodiments, the oligonucleotide detection reagent comprises, in 3′ to 5′ order: the TAC, the amplification primer, the hybridization region, the amplification blocker, and the secondary TAC. In embodiments, the secondary TAC is positioned adjacent to the amplification blocker on the oligonucleotide detection reagent. Secondary TACs, secondary targeting agents, and binding surfaces are further described herein.

In embodiments, the composition and/or kit further comprises a second nuclease. In embodiments, the second nuclease is Csm6. In embodiments where the composition and/or kit comprises a second nuclease, the oligonucleotide detection reagent further comprises a second nuclease cleavage site. In embodiments, the second nuclease cleavage site is positioned between the amplification primer and the amplification blocker. Second nucleases, e.g., Csm6, and second nuclease cleavage sites are further described herein.

In embodiments, the detection surface comprises an anchoring reagent. In embodiments, the kit provides an anchoring reagent and a reagent for immobilizing the anchoring reagent onto the detection surface. In embodiments, the anchoring reagent comprises the sequence AAGAGAGTAGTACAGCAGCCGTCAA (SEQ ID NO:66). Methods of immobilizing anchoring reagents onto a detection surface are provided herein. In embodiments, the detection surface comprises a plurality of distinct binding domains, and the targeting agent and anchoring reagent are located on two distinct binding domains on the surface. In embodiments, the detection surface comprises a plurality of distinct binding domains, and the targeting agent and anchoring reagent are located on the same binding domain on the detection surface. In embodiments, each distinct binding domain is positioned about 10 μm to about 100 μm apart from an adjacent distinct binding domain on the detection surface. In embodiments, each distinct binding domain is positioned less than 100 μm, less than 50 μm apart, or less than 10 μm apart from an adjacent distinct binding domain on the detection surface. Detection surfaces are further described herein.

### Composition and Kit, Embodiment III

In embodiments, the invention provides an oligonucleotide detection reagent comprising: (i) a primary targeting agent complement (primary TAC); (ii) a secondary targeting agent complement (secondary TAC); and (iii) a detectable label. In embodiments, the invention further provides a composition comprising the oligonucleotide detection reagent, a site-specific nuclease, and a nucleic acid of interest.

In embodiments, the invention provides a kit for detecting a nucleic acid of interest, comprising, in one or more containers, vials, or compartments: (a) an oligonucleotide detection reagent that comprises (i) a primary targeting agent complement (primary TAC); (ii) a secondary targeting agent complement (secondary TAC); and (iii) a detectable label; and (b) a site-specific nuclease having collateral activity. In embodiments, the kit further comprises one or both of a binding surface comprising a secondary targeting agent and a detection surface comprising a primary targeting agent.

Oligonucleotide detection reagents are further described herein. In embodiments, the oligonucleotide detection reagent comprises a single-stranded oligonucleotide. In embodiments, the oligonucleotide detection reagent comprises RNA. In embodiments, the oligonucleotide detection reagent comprises ssDNA. In embodiments, the oligonucleotide detection reagent comprises, in 5′ to 3′ order, the secondary TAC, the primary TAC, and the detectable label. In embodiments, the oligonucleotide detection reagent comprises, in 3′ to 5′ order, the secondary TAC, the primary TAC, and the detectable label. In embodiments, the oligonucleotide detection reagent is about 20 to about 300, about 25 to about 280, about 30 to about 250, about 35 to about 220, about 40 to about 200, about 45 to about 180, about 50 to about 150, about 55 to about 120, about 60 to about 100, or about 65 to about 80 nucleotides in length.

In embodiments, the secondary TAC is a binding partner of a secondary targeting agent on a binding surface. In embodiments, the secondary targeting agent and the binding surface are provided separately in the kit, and the kit further comprises a reagent for immobilizing the secondary targeting agent onto the binding surface. In embodiments, the secondary TAC and the secondary targeting gent comprise a binding pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the secondary targeting agent and secondary TAC are cross-reactive moieties, e.g., thiol and maleimide or iodoacetamide; aldehyde and hydrazide; or azide and alkyne or cycloalkyne. In embodiments, the secondary TAC comprises biotin, and the secondary targeting agent comprises avidin or streptavidin. In embodiments, the secondary TAC is an oligonucleotide of about 5 to about 100, about 6 to about 90, about 7 to about 80, about 8 to about 70, about 9 to about 60 nucleotides, about 10 to about 50, about 15 to about 45, about 20 to about 40, about 20 to about 30, or about 30 to about 35 nucleotides in length. Secondary TACs, secondary targeting agents, and binding surfaces are further described herein.

In embodiments, the primary TAC is a binding partner of a primary targeting agent on a detection surface. In embodiments, the targeting agent and the detection surface are provided separately in the kit, and the kit further comprises a reagent for immobilizing the targeting agent onto the detection surface. In embodiments, the primary TAC and the primary targeting agent comprise a binding pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the primary targeting agent and the primary TAC are cross-reactive moieties, e.g., thiol and maleimide or iodoacetamide; aldehyde and hydrazide; or azide and alkyne or cycloalkyne. In embodiments, the primary TAC and the primary targeting agent comprise complementary oligonucleotides. In embodiments, the primary TAC and the primary targeting agent are at least 90%, at least 95%, at least 98%, at least 99%, or 100% complementary. In embodiments, the primary TAC is an oligonucleotide of about 5 to about 100, about 6 to about 90, about 7 to about 80, about 8 to about 70, about 9 to about 60 nucleotides, about 10 to about 50, about 15 to about 45, about 20 to about 40, about 20 to about 30, about 20 to about 35, or about 30 to about 35 nucleotides in length. In embodiments, the primary TAC comprises any of SEQ ID NOs:68-71. In embodiments, the primary TAC and the primary targeting agent are substantially unreactive with the secondary TAC and the secondary targeting agent. Primary targeting agents, TACs, and detection surfaces are further described herein.

In embodiments, the detectable label is detectable by light scattering, optical absorbance, fluorescence, chemiluminescence, electrochemiluminescence (ECL), bioluminescence, phosphorescence, radioactivity, magnetic field, or combinations thereof. In embodiments, the detectable label comprises phycoerythrin (PE). In embodiments, the detectable label comprises a β-galactosidase (β-gal) enzyme that can be detected by fluorescence detection when the β-gal enzyme cleaves a substrate such as resorufin-β-D-galactopyranoside to yield a fluorescent signal. In embodiments, the detectable label comprises an ECL label. ECL labels are further described herein.

In embodiments, the site-specific nuclease is a Cas nuclease. Cas nucleases having collateral activity are further described herein. In embodiments, the Cas nuclease is a Cas12 or Cas13 nuclease. In embodiments, the composition and/or kit further comprises a guide polynucleotide, e.g., a guide RNA. Guide polynucleotides are further described herein. In embodiments, the guide RNA comprises one or both of a tracrRNA and a crRNA. In embodiments, the guide polynucleotide comprises a complementary sequence to the nucleic acid of interest. In embodiments, the guide polynucleotide is capable of forming with a complex with the site-specific nuclease. In embodiments, the site-specific nuclease in the composition and/or the kit is complexed with the guide polynucleotide.

In embodiments, the oligonucleotide detection reagent further comprises a nuclease cleavage site. In embodiments, the nuclease cleavage site comprises a sequence at which the site-specific nuclease preferentially cleaves during collateral cleavage. In embodiments, the nuclease cleavage site comprises a poly ribouridine (rU) sequence. In embodiments, the poly rU sequence comprises at least or about 2, at least or about 3, at least or about 4, at least or about 5, at least or about 6, at least or about 7, at least or about 8, at least or about 9, or at least or about 10 rU nucleotides. In embodiments, the nuclease cleavage site comprises an RNA dinucleotide. In embodiments, the nuclease cleavage site is positioned between the amplification primer and the amplification blocker. Nuclease cleavage sites are further described herein.

In embodiments where the site-specific nuclease is Cas12 and the oligonucleotide detection reagent is ssDNA, each of the primary TAC and the amplification primer further comprises a nuclease-resistant nucleotide. Nuclease-resistant nucleotides are further described herein. In embodiments, the nuclease-resistant nucleotide comprises a 2′-O-Methyl (2′OMe) moiety, a 2′-O-(2-Methoxyethyl) (2′MOE) moiety, a locked nucleic acid (LNA), a phosphorothioate linkage, a 2′-fluoro moiety, or a combination thereof.

In embodiments, the composition and/or kit further comprises a second nuclease. In embodiments, the second nuclease is Csm6. In embodiments where the composition and/or kit comprises a second nuclease, the oligonucleotide detection reagent further comprises a second nuclease cleavage site. In embodiments, the second nuclease cleavage site is positioned between the amplification primer and the amplification blocker. Second nucleases, e.g., Csm6, and second nuclease cleavage sites are further described herein.

In embodiments, the invention provides a kit for detecting multiple nucleic acids of interest, the kit comprising, in one or more containers, vials, or compartments, a plurality of oligonucleotide detection reagents, wherein an oligonucleotide detection for each unique nucleic acid of interest comprises a unique primary TAC and a unique nuclease cleavage site; and a plurality of site-specific nucleases, wherein each site-specific nuclease recognizes a unique nuclease cleavage site. In embodiments, the plurality of site-specific nucleases comprises LwaCas13a, CcaCas13b, LbaCas13a, and PsmCas13b. In embodiments, the detection surface comprises a plurality of primary targeting agents, wherein each primary targeting agent is a binding partner of a unique primary TAC. In embodiments, the detection surface comprises multiple binding domains, wherein each binding domain comprises a unique primary targeting agent. Methods for detecting multiple nucleic acids of interest, including multiplexed methods, are further described herein.

### Composition and Kit, Embodiment IV

In embodiments, the invention provides an oligonucleotide detection reagent comprising: (i) a targeting agent complement (TAC); (ii) a targeting agent blocker that is complementary to at least a portion of the TAC; (iii) a nuclease cleavage site; and (iv) a detectable label. In embodiments, the invention further provides a composition comprising the oligonucleotide detection reagent, a site-specific nuclease, and a nucleic acid of interest.

In embodiments, the invention provides a kit for detecting a nucleic acid of interest, comprising, in one or more containers, vials, or compartments: (a) an oligonucleotide detection reagent that comprises (i) a targeting agent complement (TAC); (ii) a targeting agent blocker that is complementary to at least a portion of the TAC; (iii) a nuclease cleavage site; and (iv) a detectable label; and (b) a site-specific nuclease having collateral activity. In embodiments, the kit further comprises (c) a detection surface comprising a targeting agent.

In embodiments, the invention provides a kit for detecting a nucleic acid of interest, comprising, in one or more containers, vials, or compartments: (a) an oligonucleotide detection reagent that comprises (i) a TAC; (ii) a targeting agent blocker that is complementary to at least a portion of the TAC; (iii) a nuclease cleavage site; and (iv) an amplification primer; (b) a site-specific nuclease having collateral activity; and (c) a detection surface comprising a targeting agent.

Oligonucleotide detection reagents are further described herein. In embodiments, the oligonucleotide detection reagent comprises a single-stranded oligonucleotide. In embodiments, the oligonucleotide detection reagent comprises RNA. In embodiments, the oligonucleotide detection reagent comprises ssDNA. In embodiments, the oligonucleotide detection reagent comprises, in 5′ to 3′ order, the targeting agent blocker, the nuclease cleavage site, the TAC, and the detectable label. In embodiments, the oligonucleotide detection reagent comprises ssDNA. In embodiments, the oligonucleotide detection reagent comprises, in 3′ to 5′ order, the targeting agent blocker, the nuclease cleavage site, the TAC, and the detectable label. In embodiments, the oligonucleotide detection reagent comprises, in 5′ to 3′ order, targeting agent blocker, the nuclease cleavage site, the TAC, and the amplification primer. In embodiments, the oligonucleotide detection reagent comprises, in 3′ to 5′ order, targeting agent blocker, the nuclease cleavage site, the TAC, and the amplification primer.

In embodiments, the oligonucleotide detection reagent is about 20 to about 300, about 25 to about 280, about 30 to about 250, about 35 to about 220, about 40 to about 200, about 45 to about 180, about 50 to about 150, about 55 to about 120, about 60 to about 100, or about 65 to about 80 nucleotides in length. In embodiments, the TAC is an oligonucleotide of about 5 to about 100, about 6 to about 90, about 7 to about 80, about 8 to about 70, about 9 to about 60 nucleotides, about 10 to about 50, about 15 to about 45, about 20 to about 40, about 20 to about 30, about 20 to about 35, or about 30 to about 35 nucleotides in length. In embodiments, the TAC comprises any of SEQ ID NOs:68-71. In embodiments, the targeting agent blocker comprises a complementary sequence to any of SEQ ID NOs:68-71. In embodiments, the TAC and the targeting agent blocker are substantially the same length. In embodiments, the TAC and the targeting agent blocker differ in length by no more than 1, 2, 3, 4, 5, 6, 7, 8, 9, or 10 nucleotides. In embodiments, the targeting agent blocker is shorter than the TAC by about 1, 2, 3, 4, 5, 6, 7, 8, 9, or 10 nucleotides. In embodiments, the TAC is shorter than the targeting agent blocker by about 1, 2, 3, 4, 5, 6, 7, 8, 9, or 10 nucleotides.

In embodiments where the oligonucleotide detection reagent comprises a single-stranded oligonucleotide, the nuclease cleavage site is capable of forming an oligonucleotide loop structure, thereby allowing the targeting agent blocker to hybridize to the TAC. In embodiments, the oligonucleotide loop structure is a hairpin loop. In embodiments, the TAC is capable of hybridizing to the targeting agent blocker. In embodiments, the nuclease cleavage site loop structure is capable of stabilizing the hybridization of the TAC and the targeting agent blocker.

In embodiments, the oligonucleotide detection reagent comprises a double-stranded oligonucleotide. In embodiments, the TAC is on a first strand of the oligonucleotide detection reagent, and the targeting agent blocker and the nuclease cleavage site are on a second strand of the oligonucleotide detection reagent. In embodiments, the targeting agent blocker comprises a first region and a second region, and the nuclease cleavage site is positioned between the first region and the second region of the targeting agent blocker; and wherein the first region and the second region of the targeting agent blocker are capable of hybridizing to first and second regions of the TAC, respectively. In embodiments, the first strand of the oligonucleotide detection reagent comprises, in 5′ to 3′ order: the first region of the targeting agent blocker, the nuclease cleavage site, and the second region of the targeting agent blocker. In embodiments, the second strand of the oligonucleotide detection reagent comprises, in 3′ to 5′ order: the second region of the TAC (which is complementary to the second region of the targeting agent blocker), the first region of the TAC (which is complementary to the first region of the targeting agent blocker), and the detectable label. In embodiments, the second strand of the oligonucleotide detection reagent comprises, in 3′ to 5′ order: the second region of the TAC, the first region of the TAC, and the amplification primer. In embodiments, the first strand of the oligonucleotide detection reagent comprises, in 3′ to 5′ order: the first region of the targeting agent blocker, the nuclease cleavage site, and the second region of the targeting agent blocker. In embodiments, the second strand of the oligonucleotide detection reagent comprises, in 5′ to 3′ order: the second region of the TAC, the first region of the TAC, and the detectable label. In embodiments, the second strand of the oligonucleotide detection reagent comprises, in 5′ to 3′ order: the second region of the TAC, the first region of the TAC, and the amplification primer. In embodiments, the TAC is capable of hybridizing to the targeting agent blocker. In embodiments, the presence of the nuclease cleavage site between the first and second regions of the targeting agent blocker is capable of stabilizing the hybridization of the TAC and the targeting agent blocker.

In embodiments, the TAC is a binding partner of a targeting agent on a detection surface. In embodiments, the targeting agent and the detection surface are provided separately in the kit, and the kit further comprises a reagent for immobilizing the targeting agent onto the detection surface. In embodiments, the TAC and the targeting agent comprise a binding pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the targeting agent and TAC are cross-reactive moieties, e.g., thiol and maleimide or iodoacetamide; aldehyde and hydrazide; or azide and alkyne or cycloalkyne. In embodiments, the TAC and the targeting agent comprise complementary oligonucleotides. In embodiments, the TAC and the targeting agent are at least 90%, at least 95%, at least 98%, at least 99%, or 100% complementary. Targeting agents and TACs are further described herein.

In embodiments, the nuclease cleavage site comprises a sequence at which the site-specific nuclease preferentially cleaves during collateral cleavage. In embodiments, the nuclease cleavage site comprises a poly ribouridine (rU) sequence. In embodiments, the poly rU sequence comprises at least or about 2, at least or about 3, at least or about 4, at least or about 5, at least or about 6, at least or about 7, at least or about 8, at least or about 9, or at least or about 10 rU nucleotides. In embodiments, the nuclease cleavage site comprises an RNA dinucleotide. Nuclease cleavage sites are further described herein.

In embodiments, the site-specific nuclease is a Cas nuclease. Cas nucleases having collateral activity are further described herein. In embodiments, the Cas nuclease is a Cas12 or Cas13 nuclease. In embodiments, the composition and/or kit further comprises a guide polynucleotide, e.g., a guide RNA. Guide polynucleotides are further described herein. In embodiments, the guide RNA comprises one or both of a tracrRNA and a crRNA. In embodiments, the guide polynucleotide comprises a complementary sequence to the nucleic acid of interest. In embodiments, the guide polynucleotide is capable of forming with a complex with the site-specific nuclease. In embodiments, the site-specific nuclease in the composition and/or the kit is complexed with the guide polynucleotide.

In embodiments where the site-specific nuclease is Cas12 and the oligonucleotide detection reagent is ssDNA, each of the TAC and the amplification primer further comprises a nuclease-resistant nucleotide. Nuclease-resistant nucleotides are further described herein. In embodiments, the nuclease-resistant nucleotide comprises a 2′-O-Methyl (2′OMe) moiety, a 2′-O-(2-Methoxyethyl) (2′MOE) moiety, a locked nucleic acid (LNA), a phosphorothioate linkage, a 2′-fluoro moiety, or a combination thereof.

In embodiments where the oligonucleotide detection reagent comprises a detectable label, the detectable label is detectable by light scattering, optical absorbance, fluorescence, chemiluminescence, electrochemiluminescence (ECL), bioluminescence, phosphorescence, radioactivity, magnetic field, or combinations thereof. In embodiments, the detectable label comprises phycoerythrin (PE). In embodiments, the detectable label comprises a β-galactosidase (β-gal) enzyme that can be detected by fluorescence detection when the β-gal enzyme cleaves a substrate such as resorufin-β-D-galactopyranoside to yield a fluorescent signal. In embodiments, the detectable label comprises an ECL label. ECL labels are further described herein.

In embodiments where the oligonucleotide detection reagent comprises an amplification primer, the amplification primer comprises a primer for polymerase chain reaction (PCR), ligase chain reaction (LCR), strand displacement amplification (SDA), self-sustained synthetic reaction (3SR), or an isothermal amplification method. In embodiments, the amplification primer comprises a primer for an isothermal amplification method. In embodiments, the isothermal amplification method is helicase-dependent amplification. In embodiments, the isothermal amplification method is rolling circle amplification (RCA). In embodiments, the amplification primer is about 10 to about 50, about 11 to about 45, about 12 to about 40, about 13 to about 35, about 14 to about 30, about 15 to about 40, about 20 to about 35, about 25 to about 30, or about 30 to about 35 nucleotides in length. In embodiments, the amplification primer is about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In embodiments, the amplification primer comprises the sequence GACAGAACTAGACAC (SEQ ID NO:64). Amplification primers and methods are further described herein.

In embodiments where the oligonucleotide detection reagent comprises an amplification primer, the detection surface further comprises an anchoring reagent. In embodiments, the kit provides an anchoring reagent and a reagent for immobilizing the anchoring reagent onto the detection surface. Detection surfaces, e.g., comprising anchoring reagents, are further described herein.

### Kit Components

In embodiments, the detection surface of the kit comprises a particle. In embodiments, the detection surface is a particle that is a paramagnetic bead. In embodiments, the detection surface comprises a well of multi-well plate. In embodiments, the detection surface comprises a cartridge. In embodiments, the detection surface comprises an electrode, e.g., for generating an electrochemiluminescence signal as described herein. In embodiments, the electrode is a carbon ink electrode. In embodiments, the kit further comprises a particle array.

In embodiments where the kit comprises a binding surface, the binding surface comprises a planar substrate. In embodiments, the binding surface comprises a multi-well plate. In embodiments, the binding surface comprises a particle. In embodiments, the binding surface comprises a magnet. In embodiments, the binding surface is a particle that is a paramagnetic bead. In embodiments, the kit further comprises a device for separating and/or collecting the binding surface from a reaction mixture.

In embodiments, the components of the kit, e.g., oligonucleotide binding reagent, oligonucleotide detection reagent, anchoring reagent, targeting agent, secondary targeting agent, site-specific nuclease, second nuclease, or a combination thereof, are provided lyophilized. In embodiments, the components of the kit, e.g., oligonucleotide binding reagent, oligonucleotide detection reagent, anchoring reagent, targeting agent, secondary targeting agent, site-specific nuclease, second nuclease, or a combination thereof, are provided in solution. In embodiments, each component of the kit is provided in a separate container, vial, or compartment. In embodiments, each component of the kit is provided separately according to its optimal shipping or storage temperature.

In embodiments, the kit further comprises a calibration reagent. In embodiments, the calibration reagent comprises a known quantity of a control nucleic acid. In embodiments, the kit further comprises a control oligonucleotide binding reagent that comprises a hybridization region complementary to the control nucleic acid. In embodiments, the kit further comprises a control oligonucleotide detection reagent is that known to be collaterally cleaved by the site-specific nuclease upon binding and/or cleavage of the control nucleic acid. In embodiments, the kit comprises multiple calibration reagents comprising a range of concentrations of the control nucleic acid. In embodiments, the multiple calibration reagents comprise concentrations of the control nucleic acid near the upper and lower limits of quantitation for the method. In embodiments, the multiple calibration reagents span the entire dynamic range of the method. In embodiments, the calibration reagent is a positive control reagent. In embodiments, the calibration reagent is a negative control reagent. In embodiments, the positive or negative control reagent is used to provide a basis of comparison for the sample to be assayed with the methods of the present invention.

In embodiments, the kit further comprises a polymerase, a ligase, a labeled probe, a template oligonucleotide, a buffer, a co-reactant, a blocking agent, a diluent, a stabilizing agent, a calibration agent, an assay consumable, an electrode, or a combination thereof.

In embodiments, the kit further comprises a template oligonucleotide and/or a polymerase, e.g., for performing polymerase chain reaction (PCR), ligase chain reaction (LCR), strand displacement amplification (SDA), self-sustained synthetic reaction (3SR), and/or isothermal amplification (such as, e.g., helicase-dependent amplification or rolling circle amplification). In embodiments, the template oligonucleotide for RCA comprises the sequence GTTCTGTCATATTTCAGTGAATGCGAGTCCGTCTAAGAGAGTAGTACAGCAAGAGTGTCTA (SEQ ID NO:65). In embodiments, the kit further comprises a ligase, e.g., for ligating the template oligonucleotide. In embodiments, the kit further comprises a reverse transcriptase and/or an RNA polymerase.

In embodiments, the kit further comprises a labeled probe. In embodiments, the labeled probe comprises a detectable label and a complementary sequence to an extended sequence as described herein. In embodiments, the labeled probe comprises the sequence CAGTGAATGCGAGTCCGTCTAAG (SEQ ID NO:67). Detectable labels are further described herein. In embodiments, the detectable label is an ECL label.

In embodiments, the kit further comprises a buffer, e.g., an assay buffer, a hybridization buffer, a reconstitution buffer, a wash buffer, a storage buffer, a read buffer, or a combination thereof. Hybridization buffer that can be used to provide the appropriate conditions (e.g., stringent conditions) for hybridization of complementary oligonucleotides. In embodiments, the hybridization buffer includes a nucleic acid denaturant such as formamide. In embodiments, the hybridization buffer is provided as two separate components that can be combined to form the hybridization buffer.

In embodiments, the kit further comprises a read buffer comprising a co-reactant, e.g., for performing an electrochemiluminescence measurement. Exemplary co-reactants are described, e.g., in WO 2020/142313 and U.S. Pat. Nos. 6,919,173; 7,288,410; 7,491,540; and 8,785,201.

In embodiments, the kit further comprises a blocking agent, e.g., to decrease non-specific interactions or assay signals from components in the sample that may interfere with the methods described herein. In embodiments, the kit further comprises a diluent for one or more components of the kit. In embodiments, a kit comprising the components above includes stock concentrations of the components that are 5×, 10×, 20×, 30×, 40×, 50×, 60×, 70×, 80×, 90×, 100×, 125×, 150× or higher fold concentrations of a working concentration for the methods provided herein. In embodiments, the kit further comprises a stabilizing agent, e.g., for storage of one or more components of the kit.

In embodiments, the kit further comprises an assay consumable, e.g., assay modules, vials, tubes, liquid handling and transfer devices such as pipette tips, covers and seals, racks, labels, and the like. In embodiments, the kit further comprises an assay instrument and/or instructions for carrying out the methods described herein.

All references cited herein, including patents, patent applications, papers, textbooks and the like, and the references cited therein, to the extent that they are not already, are hereby incorporated herein by reference in their entirety.

### EXAMPLES

### Example 1. Detection of RNA Using Cas13 and Oligonucleotide Detection Reagent with Amplification Primer

Reagents and assay components for a Cas13-based nucleic acid detection and amplification assay, as described in Assay Embodiment II herein, are as follows:

Cas13 Reagents

Cas13 proteins from Leptotrichia wadeii (LwaCas13a), Capnocytophaga canimorsus Cc5 (CcaCas13b), Lachnospiraceae bacterium NK4A179 (LbaCas13a), and Prevotella sp. MA2016 (PsmCas13b) are produced as described by Gootenberg et al., Science 356(6336):438-442 (2017) and Gootenberg et al., Science 360(6387):439-444 (2018). LwaCas13a is also available from Molecular Cloning Laboratories (South San Francisco, CA).

RNA for the assay is made synthetically (IDT), generated in vitro, or isolated from tissue and or clinical samples such as nasal swabs, saliva, blood and stool using established methods and kits. RNA is extracted from samples using QIAAMP® Viral RNA Mini Kit (QIAGEN) with carrier RNA according to the manufacturer's instructions. cDNA is produced using SUPERSCRIPT™ III (Invitrogen) and random hexamer primers according to manufacturer's instructions. RNA-DNA duplexes are degraded with RNase H. cDNA is stored at −70° C. until use.

Target RNA (Zika Virus)

Zika virus is purchased from ZeptoMetrix Corp. Zika virus strain PRVABC59 purified virus lysate (Cat #0810525) is used for testing crRNA ZIKV1 and crRNA ZIKV2. Zika virus (PRVABC59) was also collected from a human serum specimen in December of 2015 from Puerto Rico; NCBI Accession No. KU501215.

A Zika virus model assay is tested on a synthetic RNA of 121 bases (corresponding to nucleotides 7220-7340 of the viral genome) based on the Zika virus reference sequence ZIKV/H. sapiens/Brazil/Natal/2015, GenBank NC_035889.1.

A synthetic model Zika RNA target has the following sequence:

(SEQ ID NO: 1)
ACAAUUAACACCCCUGACCCUAAUAGUGGCCAUCAUUUUGCUCGUGGCG
CACUACAUGUACUUGAUCCCAGGGCUGCAGGCAGCAGCUGCGCGUGCUG
CCCAGAAGAGAACGGCAGCUGGCAUCAUGAAGAACCCUGUUGUGGAUGG
AAUAGUGGUGACUGACAUUGACACAAUGACAAUUGACCCCCAAGUGGAG
AAAAAGAUGGGACAGGUGCUACUCA

Recombinant polymerase amplification (RPA) reaction for amplification of the viral RNA is performed using TWIST-DX™ reverse transcriptase (RT)-RPA kits according to the manufacturer's instructions. Zika virus is amplified using RPA primers RP819 and RP821 as described by Gootenberg et al., Science 356(6336):438-442 (2017). Sequences of RP819 and RP821 are provided below:

RP819:
(SEQ ID NO: 2)
gaaatTAATACGACTCACTATAG GGCGTGGCGCACTACATGTACT
RP821:
(SEQ ID NO: 3)
TGTCAATGTCAGTCACCACTATT CCATCCA

Following the RPA reaction, target RNA for the Cas13 assay is generated using T7 RNA polymerase.

Guide RNAs (crRNA)

Guide RNAs are produced synthetically (IDT) or in vitro using methods as described in Gootenberg et al., Science 356(6336):438-442 (2017), by combining the Cas13-specific direct repeat sequence with a spacer sequence targeting the RNA of interest.

The following crRNAs are generated to target the synthetic Zika viral genome RNA of SEQ ID NO:1:

crRNA ZIKV1, targeting Zika virus genome position 7250-7277 (strain ZIKV/H. sapiens/Brazil/Natal/2015 GenBank NC_035889.1):

(SEQ ID NO: 4)
GAUUUAGACUACCCCA AAAACGAAGGGGACUAAAA C-
CAUGUAGUGCGCCACGAG CAAAAUGAUG

crRNA ZIKV2, targeting Zika virus genome position 7277-7304 (ZIKV/H. sapiens/Brazil/Natal/2015 GenBank NC_035889.1):

(SEQ ID NO: 5)
GAUUUAGACUACCCCA AAAACGAAGGGGACUAAAA C-
UGCUGCCUGCAGCCCUGG GAUCAAGUAC

The underlined portion in SEQ ID NOs:4 and 5 indicate the spacer sequence targeting the Zika viral genomic RNA. The non-underlined portions in SEQ ID NOs:4 and 5 indicate the Cas13-specific direct repeat sequence.

Oligonucleotide Detection Reagent with Rolling Circle Amplification (RCA) Primer

Oligonucleotide detection reagents containing an RCA primer are produced. The oligonucleotide detection reagents include an amplification blocker to prevent polymerase extension and protect the 3′ end from nuclease degradation (IDT) and a selective nuclease cleavage RNA dinucleotide site for LwaCas13a, rArU.

Targeting agent complements (TAC) that can be used with the oligonucleotide detection reagents include the following sequences:

TAC-1:
(SEQ ID NO: 68)
ACTGGTAACCC AGACATGATCGGT
TAC-2:
(SEQ ID NO: 69)
CTAATAGCTCCTGTGCCCTCGTAT
TAC-3:
(SEQ ID NO: 70)
AATCCGTCGACTAGCCTGAGAATT
TAC-4
(SEQ ID NO: 71)
CGTACCATTGAATCTGGAGACCTT

The sequences of oligonucleotide detection reagents are provided in Table 4.

TABLE 4
Oligonucleotide Detection Reagent Sequences with Amplification Blockers

| ID Name | Sequence | Note |
|---|---|---|
| RCA | /5Biosg/GACAGAACTAGACAC rUrUrUrUrU GC | 2′-O-methyl |
| Lwa-1 | mUmUmUmU (SEQ ID NO: 6) | uridine |
| RCA | /5Biosg/GACAGAACTAGACAC rUrUrUrUrUrUrUrUrUrU | amplification |
| Lwa-2 | rUrUrUrU mUmUmUmU (SEQ ID NO: 7) | blocker |
| RCA | /5Biosg/GACAGAACTAGACAC rArU GC mUmUmUmU | |
| Lwa-3 | (SEQ ID NO: 8) | |
| RCA | /5Biosg/GACAGAACTAGACAC rArU GC/3InvdT/ | 3′ Inverted dT |
| Lwa-4 | (SEQ ID NO: 9) | amplification |
| | | blocker |
| RCA | /5Biosg/GACAGAACTAGACAC rArU GC | Digoxigenin |
| Lwa-5 | mUmUmU/3Dig_N/(SEQ ID NO: 10) | amplification |
| | | blocker |
| RCA | /5Biosg/GACAGAACTAGACAC rUrUrUrUrU GC | |
| Lwa-6 | mUmUmU/3Dig_N/(SEQ ID NO: 11) | |
| RCA | 5′ ACTGGTAACCCAGACATGATCGGT- | 3′ Biotin blocker |
| Lwa-7 | GACAGAACTAGACAC rArU GC mUmUmU/3Bio/ | Targeting agent |
| | (SEQ ID NO: 12) | complement |
| | | (TAC)-1 |
| RCA | 5′ ACTGGTAACCCAGACATGATCGGT- | |
| Lwa-8 | GACAGAACTAGACAC rUrUrUrUrU GC mUmUmU/3Bio/ | |
| | (SEQ ID NO: 13) | |
| RCA | 5′ CTAATAGCTCCTGTGCCCTCGTAT- | 3′ Biotin blocker |
| Lwa-9 | GACAGAACTAGACAC rArU GC mUmUmU/3Bio/ | TAC-2 |
| | (SEQ ID NO: 14) | |
| RCA | 5′ CTAATAGCTCCTGTGCCCTCGTAT- | |
| Lwa-10 | GACAGAACTAGACAC rUrUrUrUrU GC mUmUmU/3Bio/ | |
| | (SEQ ID NO: 15) | |
| RCA | 5′ AATCCGTCGACTAGCCTGAGAATT- | 3′ Biotin |
| Lwa-11 | GACAGAACTAGACAC rArU GC mUmUmU/3Bio/ | amplification |
| | (SEQ ID NO: 16) | blocker |
| | | TAC-3 |
| RCA | 5′ AATCCGTCGACTAGCCTGAGAATT- | |
| Lwa-12 | GACAGAACTAGACAC rUrUrUrUrU GC mUmUmU/3Bio/ | |
| | (SEQ ID NO: 17) | |
| RCA | 5′ CGTACCATTGAATCTGGAGACCTT- | 3′ Biotin |
| Lwa-13 | GACAGAACTAGACAC rArU GC mUmUmU/3Bio/ | amplification |
| | (SEQ ID NO: 18) | blocker |
| | | TAC-4 |
| RCA | 5′ CGTACCATTGAATCTGGAGACCTT- | |
| Lwa-14 | GACAGAACTAGACAC rUrUrUrUrU GC mUmUmU/3Bio/ | |
| | (SEQ ID NO: 19) | |

RCA Reagents

The circular template for ligation and extension using the RCA primer of the oligonucleotide detection reagent (“Circ”) has the following sequence:

(SEQ ID NO: 20)
/5Phos/GTTCTGTCATATTTCAGTGAATGCGAGTCC
GTCTAAGAGAGTAGTACAGCAAGAGTGTCTA-3′

The labeled probe for the extended sequence produced by the RCA reaction has the following sequence:

(SEQ ID NO: 21)
CAGTGAATGCGAGTCCGTCTAAG/iAmMC6T/
iSp18/iAmMC6T/iSp18/3AmMO/

The labeled probe is labeled with SULFO-TAG NHS Ester (Meso Scale Discovery), followed by HPLC or FPLC purification on a size exclusion chromatography column.

The detection surface for the RCA assay is prepared by coating MULTI-ARRAY™ 96 Sm Spot Plate (Meso Scale Discovery, Rockville, MD) with 0.55 μL of streptavidin at 500 μg/mL and anchoring reagent (AAGAGAGTAGTACAGCAGCCGTCAA/3ThioMC3-D/(SEQ ID NO:22), deprotected to generate the free thiol) at 100 nM to 900 nM, for example 400 nM. The plate is dried, washed with phosphate buffer solution (PBS), and stored with desiccant at 4° C.

Assay Protocol

Viral RNA samples (synthetic, isolated directly from sample, generated via an RPA reaction as described above, or generated indirectly via a DNA template and in vitro transcription as described above) are incubated with a Cas13-crRNA complex specific for the RNA target of interest.

Each 100 μL reaction contains 45 nM purified LwaCas13a, 22.5 nM crRNA, 0.1 nM to 1.0 nM oligonucleotide detection reagent (approximately 5 to 50 fmoles/well), 0.5 μL murine RNase inhibitor (New England Biolabs), 25 ng background total human RNA (purified from HEK293FT cell culture), and varying amounts of the input target RNA in nuclease assay buffer (20 mM HEPES, 60 mM NaCl, 6 mM MgCl₂, pH 6.8). Reactions are allowed to proceed for 1 to 3 hours at 37° C.

During the Cas13 incubation, an assay plate coated with streptavidin and anchoring reagent as described above is blocked for 1 hour with a blocking solution (e.g., MSD® Blocker A (Meso Scale Discovery)) and washed.

Following the incubation of samples with Cas13, 50 μL of the reactions are added to the blocked assay plate and incubated for 1 hour at 27° C., followed by washing with PBS. The washed plate is then subjected to the RCA reaction as described in U.S. Pat. No. 10,114,015. Briefly, a ligation mix is added to each well including the following components:

(SEQ ID NO: 20)
Ligation buffer, ATP (1 mM), T4 DNA
ligase (0.15 U/μL), and Circ (4 nM;
/5Phos/GTTCTGTCATATTTCAGTGAATGCGAGTC
CGTCTAAGAGAGTAGTACAGCAAGAGTGTCTA-3′

The plate is incubated with the ligation mix for 30 minutes at room temperature, washed, and incubated with 50 μL of dATP, dGTP, dCTP, and dTTP (250 μM of each), Phi29 DNA polymerase (0.125 U/ml) and 6.25 nM labeled probe as described above. Following the RCA reaction, the plate is washed, 150 μL of read buffer is added (e.g., MSD GOLD® Read Buffer A (Meso Scale Discovery)), and the plate is read on a plate reader (e.g., MSD SECTOR® 6000 Reader (Meso Scale Discovery)).

### Example 2. Detection of RNA Using Cas13 and Oligonucleotide Detection Reagent with Amplification Primer and Digoxigenin Modifications

The oligonucleotide detection reagents that contain a digoxigenin amplification blocker, RCA Lwa-5 and RCA Lwa-6 (SEQ ID NOs:10 and 11), allow for removal of the uncleaved oligonucleotide detection reagent, thereby improving assay performance. The assays of Example 1 are modified with the uncleaved oligonucleotide detection reagent removal step as follows:

Each 100 μL reaction contains 45 nM purified LwaCas13a, 22.5 nM crRNA, 4 nM to 40 nM oligonucleotide detection reagent containing 3′ digoxigenin (RCA Lwa-5 or RCA Lwa-6, approximately 0.2 to 2 pmoles/well), 0.5 μL murine RNase inhibitor (New England Biolabs), 25 ng background total human RNA (purified from HEK293FT cell culture), and varying amounts of the input target RNA in nuclease assay buffer (20 mM HEPES, 60 mM NaCl, 6 mM MgCl₂, pH 6.8). Reactions are allowed to proceed for 1 to 3 hours at 37° C.

During the Cas13 incubation, an assay plate coated with streptavidin and anchoring reagent as described in Example 1 is blocked for 1 hour with a blocking solution (e.g., MSD® Blocker A (Meso Scale Discovery, Rockville, MD)) and washed.

Following the incubation of the samples with Cas13, 300 μg of magnetic beads (e.g., DYNABEADS™ M-270 Epoxy (ThermoFisher)) coated with anti-digoxigenin antibodies (SigmaAldrich) are added to the sample reaction mixtures to bind uncleaved oligonucleotide detection reagent. This mixture incubated for 1 hour with shaking at 37° C.

Following the incubation of the samples with the magnetic beads, the beads are removed from the sample reaction mixtures or concentrated on the side of the reaction tubes by contacting the tubes with a magnet. Following the magnetic separation, 50 μL of the reactions are added to the blocked assay plate, incubated, and washed as described in Example 1. The washed plate is then subjected to RCA and read on a plate reader, as described in Example 1.

### Example 3. Detection of RNA Using Cas13 and Oligonucleotide Detection Reagent with Amplification Primer—Single-Step Sample Amplification

A one-pot nucleic acid detection assay combines the sample DNA amplification and Cas13 incubation steps described in Example 1. The one-pot detection assay is performed in a 100 μL reaction containing 0.48 μM forward primer, 0.48 μM reverse primer, lx RPA rehydration buffer, varying amounts of input target DNA, 45 nM LwaCas13a, 22.5 nM crRNA, 125 ng background total human RNA, 0.1 nM to 1 nM oligonucleotide detection reagent, 2.5 μL murine RNase inhibitor (New England Biolabs), 2 mM ATP, 2 mM GTP, 2 mM UTP, 2 mM CTP, 1 μL T7 polymerase mix (Lucigen), 5 mM MgCl₂, and 14 mM MgAc. Reactions are allowed to proceed for 1 to 3 hours at 37° C.

During the Cas13 incubation, an assay plate coated with streptavidin and anchoring reagent as described in Example 1 is blocked for 1 hour with a blocking solution (e.g., MSD® Blocker A (Meso Scale Discovery, Rockville, MD)) and washed.

Following the incubation of the samples with Cas13, 50 μL of the reactions are added to the blocked plate, incubated, and washed as described in Example 1. The washed plate is then subjected to RCA and read on a plate reader, as described in Example 1.

### Example 4. Detection of RNA Using Cas13 and Oligonucleotide Detection Reagent with Detectable Label

Cas13 cleavage of oligonucleotide detection reagents comprising a targeting agent complement and a detectable label (e.g., ECL label) generates a cleaved labeled substrate for detection (referred to herein as “first cleaved oligonucleotide”), as described in Assay Embodiment III herein. As described herein, this approach offers the potential to multiplex the detection of differing Cas13 proteins based on their substrate specificities. A detection surface comprising multiple unique targeting agents further allows sample pooling following Cas13 incubation, which can be used to multiplex by sample or by analyte. The oligonucleotide detection reagents in this Example include a 5′ biotin followed by a nuclease cleavage RNA dinucleotide site or a poly RNA element as a substrate for the RNase activity of Cas13. The 5′ biotin allows for the removal of uncleaved oligonucleotide detection reagent, which enables specific capture of the cleaved labeled substrate following the cleavage and detection of the Cas13 activity via the detectable label (e.g., ECL label).

The oligonucleotide detection reagents provided in Table 5 are used for multiplexed assays. The poly rU region is suitable for a wide range of Cas13 proteins, including LwaCas13a, CcaCas13b, and Cas13 proteins from Bergeyella TCC 43767 (BzoCas13b), Prevotella intermedia ATCC 25611 (PinCas13b), Prevotella buccae ATCC 33574 (PbuCas13b), Prevotella intermedia (Pin2Cas13b), Porphyromonas gulae (PguCas13b), and Leptotrichia buccalis (LbuCas13a).

TABLE 5
Oligonucleotide Detection Reagent Sequences
for Labeling with ECL Label

| ID Name | Sequence | Note |
|---|---|---|
| ECL Lwa- | /5Bio/TArArUGC-ACTGGTA | Targeting |
| 1a | ACCCAGACATGATCGGT/3AmMO/ | agent |
| | (SEQ ID NO: 23) | complement |
| | | (TAC)-1 |
| ECL Lwa- | /5Bio/TA rUrUrUrUrU GCACTG | |
| 1b | GTAACCCAGACATGATCGGT/3AmMO/ | |
| | (SEQ ID NO: 24) | |
| ECL Lwa- | /5Bio/TArArUGC CTAATAGCTCC | TAC-2 |
| 3a | TGTGCCCTCGTAT/3AmMO/ | |
| | (SEQ ID NO: 25) | |
| ECL Lwa- | /5Bio/TA rUrUrUrUrU GC | |
| 3b | CTAATAGCTCCTGTGCCCTCGTAT/ | |
| | 3AmMO/ | |
| | (SEQ ID NO: 26) | |
| ECL Lwa- | /5Bio/TArArUGC | TAC-3 |
| 8a | AATCCGTCGACTAGCCTGAGAATT/ | |
| | 3AmMO/ | |
| | (SEQ ID NO: 27) | |
| ECL Lwa- | /5Bio/TA rUrUrUrUrU GC | |
| 8b | AATCCGTCGACTAGCCTGAGAATT/ | |
| | 3AmMO/ | |
| | (SEQ ID NO: 28) | |
| ECL Lwa- | /5Bio/TArArUGC | |
| 10a | CGTACCATTGAATCTGGAGACCTT/ | TAC-4 |
| | 3AmMO/ | |
| | (SEQ ID NO: 29) | |
| ECL Lwa- | /5Bio/TArUrUrUrUrUGC | |
| 10b | CGTACCATTGAATCTGGAGACCTT/ | |
| | 3AmMO/ | |
| | (SEQ ID NO: 30) | |

The oligonucleotide detection reagents are labeled with SULFO-TAG NHS Ester (Meso Scale Discovery), followed by HPLC or FPLC purification on a size exclusion chromatography column.

### Example 5. Assay with Multiplexed Samples and/or Target RNA

To perform a multiplexed assay with four samples, four individual reactions are prepared, each with an ECL-labeled oligonucleotide detection reagent specific for a different binding domain on an assay surface, wherein each binding domain includes a targeting agent sequence that is complementary to a unique TAC sequence (e.g., TAC-1, TAC-2, TAC-3, TAC-4; SEQ ID NOs:68-71). This allows each of the reactions to be pooled for ECL analysis and does not require the use of Cas13 with unique substrate specificity, providing a greater degree of multiplexing.

Each 100 μL reaction contains 45 nM purified LwaCas13a, 22.5 nM crRNA, 4 to 40 nM oligonucleotide detection reagents shown in Table 5 (0.2 to 2 pmoles/well), 0.5 μL murine RNase inhibitor (New England Biolabs), 25 ng background total human RNA (purified from HEK293FT cell culture), and varying amounts of the input target RNA in nuclease assay buffer (20 mM HEPES, 60 mM NaCl, 6 mM MgCl₂, pH 6.8). Reactions are allowed to proceed for 1 to 3 hours at 37° C.

During the Cas13 incubation, an assay plate with multiple binding domains as described above is blocked for 1 hour with a blocking solution, followed by washing and addition of 30 μL of a hybridization buffer.

Following the incubation of the samples with Cas13, 50 μg of magnetic beads (e.g., DYNABEADS™ M-270 Streptavidin (ThermoFisher)) in 15 μL of 100 mM EDTA, 1% SDS are added to bind the uncleaved oligonucleotide detection reagents. This mixture is incubated for 1 hour at room temperature with shaking.

Following the incubation of the samples with the magnetic beads, the beads are removed from the sample reaction mixtures removed or concentrated on the side of the reaction tubes by contacting the tubes with a magnet. Following the magnetic separation, 20 μL of each of the four reactions are added to the hybridization buffer in the blocked assay plates described above and incubated for 1 hour at 37° C.

Following the assay plate incubation, the plate is washed, 150 μL of read buffer is added (e.g., MSD GOLD® Read Buffer B (Meso Scale Discovery)), and the plate is read on a plate reader (e.g., MSD SECTOR® 6000 Reader (Meso Scale Discovery)).

### Example 6. Assay with Multiplexed Samples and/or Target RNA and Unique Cas13 RNA Dinucleotides

Oligonucleotide detection reagents comprising unique nuclease cleavage RNA dinucleotide site allow for Cas13 multiplexing, using the combination of four Cas13 proteins (LwaCas13a, CcaCas13b, PsmCas13b, LbaCas13a) targeting four different RNA target sequences, to multiplex the detection of up to four different RNA target sequences in a single reaction. The four different RNA target sequences may encompass sequences in four unique target RNA molecules or four target sequences within a single RNA molecule, or a combination of unique target RNA molecules and target sequences within one RNA molecule.

The oligonucleotide detection reagents provided in Table 6 are used for multiplexed assays using assay plates that have unique targeting complements (as described in Example 4) and unique Cas13 nuclease cleavage RNA dinucleotide sites.

TABLE 6
Oligonucleotide Detection Reagent Sequences
for Labeling with ECL Label

| ID Name | Sequence | Note |
|---|---|---|
| ECL Lwa- | /5Bio/TArUrCGC-ACTGGTAA | Targeting |
| 1a | CCCAGACATGATCGGT/3AmMO/ | agent |
| | (SEQ ID NO: 31) | complement |
| | | (TAC)-1 |
| ECL Cca- | /5Bio/TArArUGCCTAATAGC | TAC-2 |
| 3a | TCCTGTGCCCTCGTAT/3AmMO/ | |
| | (SEQ ID NO: 32) | |
| ECL Psm- | /5Bio/TArGrAGCAATCCGTCG | TAC-3 |
| 8a | ACTAGCCTGAGAATT/3AmMO/ | |
| | (SEQ ID NO: 33) | |
| ECL Lba- | /5Bio/TArArCGCCGTACCATT | TAC-4 |
| 10a | GAATCTGGAGACCTT/3AmMO/ | |
| | (SEQ ID NO: 34) | |

The oligonucleotide detection reagents are labeled with SULFO-TAG NHS Ester (Meso Scale Discovery), followed by HPLC or FPLC purification on a size exclusion chromatography column.

A multiplexed assay is used to detect a panel of respiratory pathogens, including SARS-CoV-2, influenza A, influenza B, and respiratory syncytial virus (RSV). Multiple crRNAs for a single pathogen can be combined to improve assay sensitivity and capture potential viral mutations. Table 7 provides crRNA spacer sequences for these respiratory pathogens. Table 8 provides the direct repeat sequences for four different Cas13 proteins. Table 9 provides crRNA for sample multiplexing generated via combinations of crRNA spacers in Table 7 with Cas13-specific direct repeat sequences in Table 8.

TABLE 7
crRNA Spacer Sequences for
Respiratory Pathogens

| ID | | |
|---|---|---|
| Name | Target | Sequence |
| COVID1 | SARS COV-2, | CAAAGCAAGAGCAGCAUCACCGCCAUUGC |
| | N | (SEQ ID NO: 35) |
| COVID2 | SARS-COV-2, | CCAACCUCUUCUGUAAUUUUUAAACUAU |
| | Orfl | (SEQ ID NO: 36) |
| COVID3 | SARS CoV-2, | GGAACUCCACUACCUGGCGUGGUUUGUA |
| | Orfl | (SEQ ID NO: 37) |
| FluA | Influenza A, | GRUCUUAUUUCUUCGGAGACAAUGCAGA |
| | NP¹ | (R = G/A) (SEQ ID NO: 38) |
| FluB | Influenza B, | UGGGUGAAUUCUAYAACCAGAUGAUGGU |
| | NP² | (Y = C/T) (SEQ ID NO: 39) |
| RSV | RSV A/B, N³ | UCAAYAUUGAGAUAGAAUCUAGAAAAUC |
| | | (Y = C/T) (SEQ ID NO: 40) |

Note¹:
The FluA spacer targets the influenza A genome segment 5 encoding the nucleocapsid protein (NP) from 1454-1481 based on the FluA NP sequence GenBank NC_026436.1.
Note²:
The FluB spacer targets the influenza B genome segment 5 encoding the nucleocapsid protein (NP) from 314-341 FluB NP sequence GenBank NC_002208.1.
Note³:
The RSV spacer targets the N-protein gene from 1513-1540 in both Human orthopneumovirus Subgroup A, GenBank Sequence ID: NC_038235.1 and Human orthopneumovirus Subgroup B, GenBank Sequence ID: NC_001781.1.

TABLE 8
Direct Repeat Sequences and Spacer
Configurations for Cas 13 Proteins

| Cas13 | Sequence |
|---|---|
| LwaCas13a | GAUUUAGACUACCCCAAAAAC |
| | GAAGGGGACUAAAAC-Spacer |
| | (SEQ ID NO: 41) |
| CcaCas13b | Spacer-GUUGGAACUGCUCU |
| | CAUUUUGGAGGGUAAUCACAAC |
| | (SEQ ID NO: 42) |
| PsmCas13b | Spacer-GUUGUAGAAGCUUA |
| | UCGUUUGGAUAGGUAUGACAAC |
| | (SEQ ID NO: 43) |
| LbaCas13a | GUUGAUGAGAAGAGCCCAAGAU |
| | AGAGGGCAAUAAC-Spacer |
| | (SEQ ID NO: 44) |

TABLE 9
crRNA for Sample Multiplexing

| Target/ | |
|---|---|
| Cas13 | Sequence |
| RSV/ | UCAAYAUUGAGAUAGAAUCUAGA |
| CcaCas | AAAUCGUUGGAACUGCUCUCAUU |
| 13b | UUGGAGGGUAAUCACAAC (SEQ ID NO: 45) |
| FluA/ | GUUGAUGAGAAGAGCCCAAGAUA |
| LbaCas | GAGGGCAAUAACGRUCUUAUUUC |
| 13a | UUCGGAGACAAUGCAGA (SEQ ID NO: 46) |
| FluB/ | UGGGUGAAUUCUAYAACCAGAUG |
| PsmCas | AUGGUGUUGUAGAAGCUUAUCGU |
| 13b | UUGGAUAGGUAUGACAAC (SEQ ID NO: 47) |
| SARS- | GAUUUAGACUACCCCAAAAACGA |
| CoV-2/ | AGGGGACUAAAACCAAAGCAAGAG |
| LwaCas | CAGCAUCACCGCCAUUGC (SEQ ID NO: 48) |
| 13a | |

The four crRNAs shown in Table 9 are combined to generate a crRNA pool, and the four oligonucleotide detection reagents shown in Table 6 are combined to form an oligonucleotide detection reagent pool for a multiplexed assay.

Each 100 μL reaction of the multiplexed assay contains 45 nM of each Cas13 protein (LwaCas13a, CcaCas13b, PsmCas13b, and LbaCas13a), 90 nM of the crRNA pool, 4 to 40 nM of the oligonucleotide detection reagent pool, 0.5 μL murine RNase inhibitor (New England Biolabs), 25 ng background total human RNA (purified from HEK293FT cell culture), and varying amounts of the input target RNA in nuclease assay buffer (20 mM HEPES, 60 mM NaCl, 6 mM MgCl₂, pH 6.8). Reactions are allowed to proceed for 1 to 3 hours at 37° C.

During the Cas13 incubation, an assay plate with multiple binding domains as described above is blocked for 1 hour with a blocking solution, followed by washing and addition of 30 μL of a hybridization buffer.

Following the incubation of the samples with Cas13, 50 to 200 μg of magnetic beads (e.g., DYNABEADS™ M-270 Streptavidin (ThermoFisher)) in 15 μL of 100 mM EDTA, 1% SDS are added to bind the uncleaved oligonucleotide detection reagents. This mixture is incubated for 1 hour at room temperature with shaking.

Following the incubation of the samples with the magnetic beads, the beads are removed from the reaction mixtures or concentrated on the side of the reaction tubes by contacting the tubes with a magnet. Following the magnetic separation, 20 μL of each reaction is added to the hybridization buffer in the blocked assay plates and incubated as described in Example 5. The washed plate is read on a plate reader, as described in Example 5.

### Example 7. Detection of RNA Using Cas13 and Oligonucleotide Detection Reagent with Hairpin Loop Structure

Oligonucleotide detection reagents that include a hairpin loop structure are described in Assay Embodiment IV and depicted in FIG. 4A herein. These oligonucleotide detection reagents comprise: a targeting agent blocker, a nuclease cleavage site (e.g., Cas13 RNase site), a targeting agent complement, and a detectable label. Exemplary oligonucleotide detection reagent sequences are shown in Table 10.

TABLE 10
Oligonucleotide Detection Reagent Sequences with Hairpin Loop

| ID Name | Sequence | Notes |
|---|---|---|
| ECL RNase | ATCATGTCTGGGTTAC rUrUrUrUrUrUrUrUrUrU | Poly rU |
| HP1 | ACTGGTAACCCAGACATGATCGGT/3AmMO/ (SEQ ID | cleavage site |
| | NO: 49) | |
| ECL RNase | CATGTCTGGGTTAC rUrUrUrUrU | Targeting |
| HP2 | ACTGGTAACCCAGACATGATCGGT/3AmMO/ (SEQ ID | |
| | NO: 50) | |
| ECL RNase | TCATGTCTGGGTTA rUrUrUrUrU | agent |
| HP3 | ACTGGTAACCCAGACATGATCGGT/3AmMO/ (SEQ ID | |
| | NO: 51) | |
| ECL RNase | TCATGTCTGGGTTA rUrUrUrUrUrUrUrUrUrU | complement |
| HP4 | ACTGGTAACCCAGACATGATCGGT/3AmMO/ (SEQ ID | |
| | NO: 52) | |
| ECL RNase | ATCATGTCTGGGTTA rUrUrUrUrU | (TAC)-1 |
| HP5 | ACTGGTAACCCAGACATGATCGGT/3AmMO/ (SEQ ID | |
| | NO: 53) | |
| ECL Lwa- | ATCATGTCTGGGTTA-TArUrCGC- | LwaCas13a |
| 1HP | ACTGGTAACCCAGACATGATCGGT/3AmMO/ (SEQ ID | cleavage site |
| | NO: 54) | TAC-1 |
| ECL Cca- | AGGGCACAGGAGCT-TArArUGC | CcaCas13b |
| 3HP | CTAATAGCTCCTGTGCCCTCGTAT/3AmMO/ (SEQ ID | cleavage site |
| | NO: 55) | TAC-2 |
| ECL Psm- | TCAGGCTAGTCGAC-TArGrAGC | PsmCas13b |
| 8HP | AATCCGTCGACTAGCCTGAGAATT/3AmMO/ (SEQ ID | cleavage site |
| | NO: 56) | TAC-3 |
| ECL Lba- | TCTCCAGATTCAAT-TArArCGC | LbaCas13a |
| 10HP | CGTACCATTGAATCTGGAGACCTT/3AmMO/ (SEQ ID | cleavage site |
| | NO: 57) | TAC-4 |

The oligonucleotide detection reagents provided in Table 10 can be used for multiplexed assays using assay plates that have unique targeting complements (as described in Example 4). The oligonucleotide detection reagents are labeled with SULFO-TAG NHS Ester (Meso Scale Discovery), followed by HPLC or FPLC purification on a size exclusion chromatography column.

Assays using the oligonucleotide detection reagents containing hairpin loops are performed using the same experimental procedures outlined in Examples 5 and 6. In brief, each 100 μL reaction contains 45 nM purified LwaCas13a, 22.5 nM crRNA, 4 to 40 nM oligonucleotide detection reagent (0.2 to 2 pmoles/well), 0.5 μL murine RNase inhibitor (New England Biolabs), 25 ng background total human RNA (purified from HEK293FT cell culture), and varying amounts of the input target RNA in nuclease assay buffer (20 mM HEPES, 60 mM NaCl, 6 mM MgCl₂, pH 6.8). Reactions are allowed to proceed for 1 to 3 hours at 37° C.

During the Cas13 incubation, an assay plate with multiple binding domains as described above is blocked for 1 hour with a blocking solution, followed by washing and addition of 30 μL of a hybridization buffer.

Following the incubation of the samples with Cas13, 20 μL of each of the four reactions are added to the hybridization buffer in the blocked assay plates described above and incubated for 1 hour at 37° C. Following the plate incubation, the plate is washed and read on a plate reader, as described in Example 5.

### Example 8. Detection of RNA Using Cas13 and Double-Stranded Oligonucleotide Detection Reagent

Double-stranded oligonucleotide detection reagents that include a nuclease cleavage site on one strand are described in Assay Embodiment IV and depicted in FIG. 4B herein. These oligonucleotide detection reagents comprise a double-stranded oligonucleotide: a first strand labeled with a detectable label (e.g., ECL label) and capable of binding to a targeting agent, and a second strand that is complementary to the first strand and comprising a nuclease cleavage site, e.g., on an exposed loop.

An exemplary first strand, containing a targeting agent complement (TAC-1) and a 3′AmMO moiety for conjugating to a SULFO-TAG NHS Ester (Meso Scale Discovery), has the following sequence:

(SEQ ID NO: 58)
ECL Str1-1: ACTGGTAACCCAGACATGATCGGT/3AmMO/

Exemplary second strand sequences of the oligonucleotide detection reagents are shown in Table 11.

TABLE 11
Oligonucleotide Detection Reagent
Second Strand Sequences

| ID Name | Sequence | Notes |
|---|---|---|
| LoopSubs | ACCGATCATGTC rUrUrUrUrU | Poly rU |
| Str2-1 | TGGGTTACCAGT | cleavage |
| | (SEQ ID NO: 59) | site |
| | ACCGATCATGTC TArUrCGC | LwaCas13a |
| LoopSubs | TGGGTTACCAGT | cleavage |
| Lwa Str2-1 | (SEQ ID NO: 60) | site |
| LoopSubs | ACCGATCATGTC TArArUGC | CcaCas13b |
| Cca Str2-1 | TGGGTTACCAGT | cleavage |
| | (SEQ ID NO: 61) | site |
| LoopSubs | ACCGATCATGTC TArGrAGC | PsmCas13b |
| Psm Str2-1 | TGGGTTACCAGT | cleavage |
| | (SEQ ID NO: 62) | site |
| LoopSubs | ACCGATCATGTC TArArCGC | LbaCas13a |
| Lba Str2-1 | TGGGTTACCAGT | cleavage |
| | (SEQ ID NO: 63) | site |

The double-stranded oligonucleotide detection reagent is prepared by adding the second strands to the first strand in excess, heated to 95° C., and slowly cooled at −1° C./min to allow the two complementary sequences to hybridize. The double-stranded oligonucleotide detection reagents are purified using column chromatography.

The double-stranded oligonucleotide detection reagents are used in the same protocols as described above for Example 7, except that the reaction mixtures are incubated at 37° C. with hybridization buffer containing 100 mM to 200 mM NaCl, 10 mM Tris-HCl (pH 7.8), 1 mM EDTA, and 0.1% SDS, to optimize the selection of RNAse degraded products over the intact substrate complexes.

What is claimed is:

1. A method for detecting a nucleic acid of interest in a sample, comprising:
a. contacting the sample with an oligonucleotide binding reagent, wherein the oligonucleotide binding reagent comprises:
i. a targeting agent complement;
ii. an amplification primer;
iii. a hybridization region comprising a complementary sequence to the nucleic acid of interest; and
iv. an amplification blocker;
b. forming a binding complex comprising the nucleic acid of interest and the oligonucleotide binding reagent;
c. contacting the binding complex with a site-specific nuclease that cleaves the oligonucleotide binding reagent to remove the amplification blocker therefrom, thereby generating a first cleaved oligonucleotide comprising the targeting agent complement and the amplification primer, wherein the first cleaved oligonucleotide is not bound to the nucleic acid of interest;
d. immobilizing the first cleaved oligonucleotide to a detection surface comprising a targeting agent, wherein the targeting agent is a binding partner of the targeting agent complement;
e. extending the first cleaved oligonucleotide on the detection surface to form an extended oligonucleotide; and
f. detecting the extended oligonucleotide, thereby detecting the nucleic acid of interest in the sample.

2. The method of claim 1, wherein:
the oligonucleotide binding reagent comprises, in 5′ to 3′ order: the targeting agent complement, the amplication primer, the hybridization region, and the amplication blocker;
(b) the amplification primer comprises a primer for polymerase chain reaction (PCR), ligase chain reaction (LCR), strand displacement amplification (SDA), self-sustained synthetic reaction (3SR), or an isothermal amplification method;
(c) the amplification blocker blocks amplification of the amplification primer;
(d) the oligonucleotide binding reagent further comprises a nuclease binding site for the site-specific nuclease:
(e) the binding complex comprises a double-stranded duplex formed by the nucleic acid of interest and the oligonucleotide binding reagent;
(f) the site-specific nuclease is a nickase, optionally wherein the nickase is a Cas9 nickase or a Cas12a nickase;
(g) the oligonucleotide binding reagent further comprises a secondary targeting agent complement, optionally wherein the targeting agent complement is at a 5′ end of the oligonucleotide binding reagent, and the secondary targeting agent complement is at a 3′ end of the oligonucleotide binding reagent:
(h) the oligonucleotide binding reagent comprises a single-stranded oligonucleotide:
(i) the targeting agent complement and the targeting agent comprise complementary oligonucleotides;
(j) the nucleic acid of interest is a single-stranded oligonucleotide or a double-stranded oligonucleotide;
(k) the detection surface further comprises an anchoring reagent immobilized thereon,
wherein the extended oligonucleotide binds the anchoring reagent, and wherein the detecting comprises measuring the amount of extended oligonucleotide bound to the detection surface:
(l) the detection surface comprises a particle or a well of a multi-well plate, optionally wherein the detection surface comprises an electrode; or
(m) combinations thereof.

3-5. (canceled)

6. The method of claim 2, wherein the nuclease binding site is positioned between the hybridization region and the amplification blocker; and/or
wherein the nuclease binding site comprises at least a portion of the hybridization region.

7. (canceled)

8 (canceled)

9 The method of any one of claims 5 te 8 claim 2, wherein the site-specific nuclease is complexed with a guide polynucleotide comprising a guide sequence,
wherein the guide sequence is capable of hybridizing to a complement of the nuclease binding site.

10. The method of claim 9, wherein the site-specific nuclease selectively generates a single-stranded cleavage in the nuclease binding site of the oligonucleotide binding reagent, thereby removing the amplification blocker to generate the first cleaved oligonucleotide.

11-14. (canceled)

15. The method of claim 2, wherein the oligonucleotide binding reagent comprises, in 5′ to 3′ order: the targeting agent complement, the amplification primer, the hybridization region, the amplification blocker, and the secondary targeting agent complement.

16. (canceled)

17. The method of claim 2, wherein;
i) the secondary targeting reagent complement is a binding partner of a secondary targeting agent on a binding surface;
ii) each of the targeting agent complement and the targeting agent is substantially unreactive with the secondary targeting agent and secondary targeting agent complement;
iii) the secondary targeting agent complement is positioned adjacent to the amplification blocker on the oligonucleotide binding reagent, such that cleavage of the oligonucleotide binding reagent by the site-specific nuclease forms a second cleaved oligonucleotide comprising the amplification blocker and the secondary targeting agent complement;
or
iv) combinations thereof.

18-20. (canceled)

21. The method of claim 17, wherein the method comprises iii) and further comprises, prior to the extending, removing the second cleaved oligonucleotide, uncleaved oligonucleotide binding reagent, or both.

22. The method of claim 21, wherein the method comprises i), and
wherein the removing comprises contacting the second cleaved oligonucleotide, uncleaved oligonucleotide binding reagent, or both, with the binding surface.

23. The method of any one of claim 1, wherein the extending comprises polymerase chain reaction (PCR), ligase chain reaction (LCR), strand displacement amplification (SDA), self-sustained synthetic reaction (3SR), or an isothermal amplification method.

24. (canceled)

25. The method of claim 23, wherein the amplification primer comprises a primer for an isothermal amplification method, wherein the extending comprises an isothermal amplification method, and wherein the isothermal amplification method comprises helicase-dependent amplification, rolling circle amplification (RCA), or both.

26-28. (canceled)

29. The method of claim 1, wherein the detecting comprises:
contacting the extended oligonucleotide with a labeled probe comprising a detectable label, wherein the labeled probe binds to the extended oligonucleotide; and
measuring the amount of labeled probe bound to the extended oligonucleotide.

30. The method of claim 29, wherein the detectable label is detectable by light scattering, optical absorbance, fluorescence, chemiluminescence, electrochemiluminescence (ECL), bioluminescence, phosphorescence, radioactivity, magnetic field, or combinations thereof.

31. (canceled)

32. The method of claim 1, wherein following step (c), the nucleic acid of interest binds to an additional copy of the oligonucleotide binding reagent.

33. The method of claim 32, further comprising, following step (c) and prior to step (d), repeating steps (a) to (c) to generate a plurality of first cleaved oligonucleotides,
wherein step (d) comprises immobilizing the plurality of first cleaved oligonucleotides to the detection surface, wherein step (e) comprises extending the plurality of first cleaved oligonucleotides on the detection surface to form a plurality of extended oligonucleotides, and wherein step (f) comprises detecting the plurality of extended oligonucleotides.

34-39 (canceled)

40. A method for detecting a nucleic acid of interest in a sample, comprising:
a. contacting the sample with a site-specific nuclease comprising collateral cleavage activity and an oligonucleotide detection reagent, wherein the oligonucleotide detection reagent comprises:
i. a targeting agent complement;
ii. an amplification primer; and
iii. an amplification blocker,
wherein the site-specific nuclease binds to the nucleic acid of interest and collaterally cleaves the oligonucleotide detection reagent to remove the amplification blocker therefrom, thereby generating a first cleaved oligonucleotide comprising the targeting agent complement and the amplification primer;
b. immobilizing the first cleaved oligonucleotide to a detection surface comprising a targeting agent, wherein the targeting agent is a binding partner of the targeting agent complement;
c. extending the first cleaved oligonucleotide to form an extended oligonucleotide; and
d. detecting the extended oligonucleotide, thereby detecting the nucleic acid of interest in the sample.

41-83. (canceled)

84. A method for detecting a nucleic acid of interest in a sample, comprising:
a. contacting the sample with a site-specific nuclease comprising collateral cleavage activity and an oligonucleotide detection reagent, wherein the oligonucleotide detection reagent comprises:
i. a primary targeting agent complement;
ii. a secondary targeting agent complement; and
iii. a detectable label;
wherein the site-specific nuclease binds to the nucleic acid of interest and collaterally cleaves the oligonucleotide detection reagent, thereby generating (i) a cleaved secondary targeting agent complement and (ii) a first cleaved oligonucleotide comprising the primary targeting agent complement and the detectable label;
b. binding the cleaved secondary targeting agent complement, uncleaved oligonucleotide detection reagent, or both, to a binding surface comprising a secondary targeting agent that is a binding partner of the secondary targeting agent complement;
c. immobilizing the first cleaved oligonucleotide to a detection surface comprising a primary targeting agent, wherein the primary targeting agent is a binding partner of the primary targeting agent complement; and
d. detecting the first cleaved oligonucleotide immobilized on the detection surface, wherein the cleaved secondary targeting agent complement and the uncleaved oligonucleotide detection reagent on the binding surface are substantially undetected, thereby detecting the nucleic acid of interest in the sample.

85-120. (canceled)

121. A method for detecting a nucleic acid of interest in a sample, comprising:
a. contacting the sample with a site-specific nuclease comprising collateral cleavage activity, and an oligonucleotide detection reagent, wherein the oligonucleotide detection reagent comprises:
i. a targeting agent complement;
ii. a targeting agent blocker that is complementary to at least a portion of the targeting agent complement;
iii. a nuclease cleavage site; and
iv. a detectable label;
wherein the targeting agent complement and the targeting agent blocker are hybridized,
wherein the site-specific nuclease binds to the nucleic acid of interest and collaterally cleaves the oligonucleotide detection reagent at the nuclease cleavage sequence, thereby (i) destabilizing hybridization of the targeting agent complement and the targeting agent blocker and (ii) generating an unblocked oligonucleotide comprising the targeting agent complement and the detectable label;
b. immobilizing the unblocked oligonucleotide to a detection surface comprising a targeting agent, wherein the targeting agent is a binding partner of the targeting agent complement, wherein uncleaved oligonucleotide detection reagent does not substantially bind to the detection surface; and
c. detecting the unblocked oligonucleotide immobilized on the detection surface, thereby detecting the nucleic acid of interest in the sample.

122-154. (canceled)

155. An oligonucleotide binding reagent comprising:
a) (i) a targeting agent complement (TAC); (ii) an amplification primer; and (iii) an amplification blocker; or
b) (i) a targeting agent complement (TAC); (ii) an amplification primer; and (iii) an amplification blocker; or
c) (i) a primary targeting agent complement (primary TAC); (ii) a secondary targeting agent complement (secondary TAC); and (iii) a detectable label; or
d) (i) a targeting agent complement (TAC); (ii) a targeting agent blocker that is complementary to at least a portion of the TAC; (iii) a nuclease cleavage site; and (iv) a detectable label.

156. A composition comprising the oligonucleotide reagent of claim 155 and one or both of a site-specific nuclease and a nucleic acid of interest.

157-160. (canceled)


SEQUENCE LISTING (71 sequences)

SEQ ID NO: 1 - RNA, 221 nt, Artificial Sequence
  source: /note=“Description of Artificial Sequence Synthetic polynucleotide”
acaauuaacaccccugacccuaauaguggccaucauuuugcucguggcgcacuacauguacuugaucccagggcugcaggcagcagcugcgcgugcugcccagaagagaacggcagcuggcaucaugaagaacccuguuguggauggaauaguggugacugacauugacacaaugacaauugacccccaaguggagaaaaagaugggacaggugcuacuca

SEQ ID NO: 2 - DNA, 45 bp, Artificial Sequence
  source: /note=“Description of Artificial Sequence Synthetic primer”
gaaattaatacgactcactatagggcgtggcgcactacatgtact

SEQ ID NO: 3 - DNA, 30 bp, Artificial Sequence
  source: /note=“Description of Artificial Sequence Synthetic primer”
tgtcaatgtcagtcaccactattccatcca

SEQ ID NO: 4 - RNA, 64 nt, Artificial Sequence
  source: /note=“Description of Artificial Sequence Synthetic oligonucleotide”
gauuuagacuaccccaaaaacgaaggggacuaaaaccauguagugcgccacgagcaaaaugaug

SEQ ID NO: 5 - RNA, 64 nt, Artificial Sequence
  source: /note=“Description of Artificial Sequence Synthetic oligonucleotide”
gauuuagacuaccccaaaaacgaaggggacuaaaacugcugccugcagcccugggaucaaguac

SEQ ID NO: 6 - DNA, 26 bp, Artificial Sequence
  source: /note=“Description of Artificial Sequence Synthetic oligonucleotide”
  source: /note=“Description of Combined DNA/RNA Molecule Synthetic oligonucleotide”
gacagaactagacacuuuuugcuuuu

SEQ ID NO: 7 - DNA, 33 bp, Artificial Sequence
  source: /note=“Description of Artificial Sequence Synthetic oligonucleotide”
  source: /note=“Description of Combined DNA/RNA Molecule Synthetic oligonucleotide”
gacagaactagacacuuuuuuuuuuuuuuuuuu

SEQ ID NO: 8 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
gacagaactagacacaugcuuuu

SEQ ID NO: 9 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gacagaactagacacaugct

SEQ ID NO: 10 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
gacagaactagacacaugcuuu

SEQ ID NO: 11 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
gacagaactagacacuuuuugcuuu

SEQ ID NO: 12 - DNA, 46 bp, Artificial Sequence
  source: /note=“Description of Artificial Sequence Synthetic oligonucleotide”
  source: /note=“Description of Combined DNA/RNA Molecule Synthetic oligonucleotide”
actggtaacccagacatgatcggtgacagaactagacacaugcuuu

SEQ ID NO: 13 - DNA, 49 bp, Artificial Sequence
  source: /note=“Description of Artificial Sequence Synthetic oligonucleotide”
  source: /note=“Description of Combined DNA/RNA Molecule Synthetic oligonucleotide”
actggtaacccagacatgatcggtgacagaactagacacuuuuugcuuu

SEQ ID NO: 14 - DNA, 46 bp, Artificial Sequence
  source: /note=“Description of Artificial Sequence Synthetic oligonucleotide”
  source: /note=“Description of Combined DNA/RNA Molecule Synthetic oligonucleotide”
ctaatagctcctgtgccctcgtatgacagaactagacacaugcuuu

SEQ ID NO: 15 - DNA, 49 bp, Artificial Sequence
  source: /note=“Description of Artificial Sequence Synthetic oligonucleotide”
  source: /note=“Description of Combined DNA/RNA Molecule Synthetic oligonucleotide”
ctaatagctcctgtgccctcgtatgacagaactagacacuuuuugcuuu

SEQ ID NO: 16 - DNA, 46 bp, Artificial Sequence
  source: /note=“Description of Artificial Sequence Synthetic oligonucleotide”
  source: /note=“Description of Combined DNA/RNA Molecule Synthetic oligonucleotide”
aatccgtcgactagcctgagaattgacagaactagacacaugcuuu

SEQ ID NO: 17 - DNA, 49 bp, Artificial Sequence
  source: /note=“Description of Artificial Sequence Synthetic oligonucleotide”
  source: /note=“Description of Combined DNA/RNA Molecule Synthetic oligonucleotide”
aatccgtcgactagcctgagaattgacagaactagacacuuuuugcuuu

SEQ ID NO: 18 - DNA, 46 bp, Artificial Sequence
  source: /note=“Description of Artificial Sequence Synthetic oligonucleotide”
  source: /note=“Description of Combined DNA/RNA Molecule Synthetic oligonucleotide”
cgtaccattgaatctggagaccttgacagaactagacacaugcuuu

SEQ ID NO: 19 - DNA, 49 bp, Artificial Sequence
  source: /note=“Description of Artificial Sequence Synthetic oligonucleotide”
  source: /note=“Description of Combined DNA/RNA Molecule Synthetic oligonucleotide”
cgtaccattgaatctggagaccttgacagaactagacacuuuuugcuuu

SEQ ID NO: 20 - DNA, 61 bp, Artificial Sequence
  source: /note=“Description of Artificial Sequence Synthetic oligonucleotide”
gttctgtcatatttcagtgaatgcgagtccgtctaagagagtagtacagcaagagtgtcta

SEQ ID NO: 21 - DNA, 24 bp, Artificial Sequence
  source: /note=“Description of Artificial Sequence Synthetic probe”
cagtgaatgcgagtccgtctaagt

SEQ ID NO: 22 - DNA, 25 bp, Artificial Sequence
  source: /note=“Description of Artificial Sequence Synthetic oligonucleotide”
aagagagtagtacagcagccgtcaa

SEQ ID NO: 23 - DNA, 30 bp, Artificial Sequence
  source: /note=“Description of Artificial Sequence Synthetic oligonucleotide”
  source: /note=“Description of Combined DNA/RNA Molecule Synthetic oligonucleotide”
taaugcactggtaacccagacatgatcggt

SEQ ID NO: 24 - DNA, 33 bp, Artificial Sequence
  source: /note=“Description of Artificial Sequence Synthetic oligonucleotide”
  source: /note=“Description of Combined DNA/RNA Molecule Synthetic oligonucleotide”
tauuuuugcactggtaacccagacatgatcggt

SEQ ID NO: 25 - DNA, 30 bp, Artificial Sequence
  source: /note=“Description of Artificial Sequence Synthetic oligonucleotide”
  source: /note=“Description of Combined DNA/RNA Molecule Synthetic oligonucleotide”
taaugcctaatagctcctgtgccctcgtat

SEQ ID NO: 26 - DNA, 33 bp, Artificial Sequence
  source: /note=“Description of Artificial Sequence Synthetic oligonucleotide”
  source: /note=“Description of Combined DNA/RNA Molecule Synthetic oligonucleotide”
tauuuuugcctaatagctcctgtgccctcgtat

SEQ ID NO: 27 - DNA, 30 bp, Artificial Sequence
  source: /note=“Description of Artificial Sequence Synthetic oligonucleotide”
  source: /note=“Description of Combined DNA/RNA Molecule Synthetic oligonucleotide”
taaugcaatccgtcgactagcctgagaatt

SEQ ID NO: 28 - DNA, 33 bp, Artificial Sequence
  source: /note=“Description of Artificial Sequence Synthetic oligonucleotide”
  source: /note=“Description of Combined DNA/RNA Molecule Synthetic oligonucleotide”
tauuuuugcaatccgtcgactagcctgagaatt

SEQ ID NO: 29 - DNA, 30 bp, Artificial Sequence
  source: /note=“Description of Artificial Sequence Synthetic oligonucleotide”
  source: /note=“Description of Combined DNA/RNA Molecule Synthetic oligonucleotide”
taaugccgtaccattgaatctggagacctt

SEQ ID NO: 30 - DNA, 33 bp, Artificial Sequence
  source: /note=“Description of Artificial Sequence Synthetic oligonucleotide”
  source: /note=“Description of Combined DNA/RNA Molecule Synthetic oligonucleotide”
tauuuuugccgtaccattgaatctggagacctt

SEQ ID NO: 31 - DNA, 30 bp, Artificial Sequence
  source: /note=“Description of Artificial Sequence Synthetic oligonucleotide”
  source: /note=“Description of Combined DNA/RNA Molecule Synthetic oligonucleotide”
taucgcactggtaacccagacatgatcggt

SEQ ID NO: 32 - DNA, 30 bp, Artificial Sequence; identical to SEQ ID NO: 25
  source: /note=“Description of Artificial Sequence Synthetic oligonucleotide”
  source: /note=“Description of Combined DNA/RNA Molecule Synthetic oligonucleotide”

SEQ ID NO: 33 - DNA, 30 bp, Artificial Sequence
  source: /note=“Description of Artificial Sequence Synthetic oligonucleotide”
  source: /note=“Description of Combined DNA/RNA Molecule Synthetic oligonucleotide”
tagagcaatccgtcgactagcctgagaatt

SEQ ID NO: 34 - DNA, 30 bp, Artificial Sequence
  source: /note=“Description of Artificial Sequence Synthetic oligonucleotide”
  source: /note=“Description of Combined DNA/RNA Molecule Synthetic oligonucleotide”
taacgccgtaccattgaatctggagacctt

SEQ ID NO: 35 - RNA, 29 nt, Artificial Sequence
  source: /note=“Description of Artificial Sequence Synthetic oligonucleotide”
caaagcaagagcagcaucaccgccauugc

SEQ ID NO: 36 - RNA, 28 nt, Artificial Sequence
  source: /note=“Description of Artificial Sequence Synthetic oligonucleotide”
ccaaccucuucuguaauuuuuaaacuau

SEQ ID NO: 37 - RNA, 28 nt, Artificial Sequence
  source: /note=“Description of Artificial Sequence Synthetic oligonucleotide”
ggaacuccacuaccuggcgugguuugua

SEQ ID NO: 38 - RNA, 28 nt, Artificial Sequence
  source: /note=“Description of Artificial Sequence Synthetic oligonucleotide”
grucuuauuucuucggagacaaugcaga

SEQ ID NO: 39 - RNA, 28 nt, Artificial Sequence
  source: /note=“Description of Artificial Sequence Synthetic oligonucleotide”
ugggugaauucuayaaccagaugauggu

SEQ ID NO: 40 - RNA, 28 nt, Artificial Sequence
  source: /note=“Description of Artificial Sequence Synthetic oligonucleotide”
ucaayauugagauagaaucuagaaaauc

SEQ ID NO: 41 - RNA, 36 nt, Artificial Sequence
  source: /note=“Description of Artificial Sequence Synthetic oligonucleotide”
gauuuagacuaccccaaaaacgaaggggacuaaaac

SEQ ID NO: 42 - RNA, 36 nt, Artificial Sequence
  source: /note=“Description of Artificial Sequence Synthetic oligonucleotide”
guuggaacugcucucauuuuggaggguaaucacaac

SEQ ID NO: 43 - RNA, 36 nt, Artificial Sequence
  source: /note=“Description of Artificial Sequence Synthetic oligonucleotide”
guuguagaagcuuaucguuuggauagguaugacaac

SEQ ID NO: 44 - RNA, 35 nt, Artificial Sequence
  source: /note=“Description of Artificial Sequence Synthetic oligonucleotide”
guugaugagaagagcccaagauagagggcaauaac

SEQ ID NO: 45 - RNA, 64 nt, Artificial Sequence
  source: /note=“Description of Artificial Sequence Synthetic oligonucleotide”
ucaayauugagauagaaucuagaaaaucguuggaacugcucucauuuuggaggguaaucacaac

SEQ ID NO: 46 - RNA, 63 nt, Artificial Sequence
  source: /note=“Description of Artificial Sequence Synthetic oligonucleotide”
guugaugagaagagcccaagauagagggcaauaacgrucuuauuucuucggagacaaugcaga

SEQ ID NO: 47 - RNA, 64 nt, Artificial Sequence
  source: /note=“Description of Artificial Sequence Synthetic oligonucleotide”
ugggugaauucuayaaccagaugaugguguuguagaagcuuaucguuuggauagguaugacaac

SEQ ID NO: 48 - RNA, 65 nt, Artificial Sequence
  source: /note=“Description of Artificial Sequence Synthetic oligonucleotide”
gauuuagacuaccccaaaaacgaaggggacuaaaaccaaagcaagagcagcaucaccgccauugc

SEQ ID NO: 49 - DNA, 50 bp, Artificial Sequence
  source: /note=“Description of Artificial Sequence Synthetic oligonucleotide”
  source: /note=“Description of Combined DNA/RNA Molecule Synthetic oligonucleotide”
atcatgtctgggttacuuuuuuuuuuactggtaacccagacatgatcggt

SEQ ID NO: 50 - DNA, 43 bp, Artificial Sequence
  source: /note=“Description of Artificial Sequence Synthetic oligonucleotide”
  source: /note=“Description of Combined DNA/RNA Molecule Synthetic oligonucleotide”
catgtctgggttacuuuuuactggtaacccagacatgatcggt

SEQ ID NO: 51 - DNA, 43 bp, Artificial Sequence
  source: /note=“Description of Artificial Sequence Synthetic oligonucleotide”
  source: /note=“Description of Combined DNA/RNA Molecule Synthetic oligonucleotide”
tcatgtctgggttauuuuuactggtaacccagacatgatcggt

SEQ ID NO: 52 - DNA, 48 bp, Artificial Sequence
  source: /note=“Description of Artificial Sequence Synthetic oligonucleotide”
  source: /note=“Description of Combined DNA/RNA Molecule Synthetic oligonucleotide”
tcatgtctgggttauuuuuuuuuuactggtaacccagacatgatcggt

SEQ ID NO: 53 - DNA, 44 bp, Artificial Sequence
  source: /note=“Description of Artificial Sequence Synthetic oligonucleotide”
  source: /note=“Description of Combined DNA/RNA Molecule Synthetic oligonucleotide”
atcatgtctgggttauuuuuactggtaacccagacatgatcggt

SEQ ID NO: 54 - DNA, 45 bp, Artificial Sequence
  source: /note=“Description of Artificial Sequence Synthetic oligonucleotide”
  source: /note=“Description of Combined DNA/RNA Molecule Synthetic oligonucleotide”
atcatgtctgggttataucgcactggtaacccagacatgatcggt

SEQ ID NO: 55 - DNA, 44 bp, Artificial Sequence
  source: /note=“Description of Artificial Sequence Synthetic oligonucleotide”
  source: /note=“Description of Combined DNA/RNA Molecule Synthetic oligonucleotide”
agggcacaggagcttaaugcctaatagctcctgtgccctcgtat

SEQ ID NO: 56 - DNA, 44 bp, Artificial Sequence
  source: /note=“Description of Artificial Sequence Synthetic oligonucleotide”
  source: /note=“Description of Combined DNA/RNA Molecule Synthetic oligonucleotide”
tcaggctagtcgactagagcaatccgtcgactagcctgagaatt

SEQ ID NO: 57 - DNA, 44 bp, Artificial Sequence
  source: /note=“Description of Artificial Sequence Synthetic oligonucleotide”
  source: /note=“Description of Combined DNA/RNA Molecule Synthetic oligonucleotide”
tctccagattcaattaacgccgtaccattgaatctggagacctt

SEQ ID NO: 58 - DNA, 24 bp, Artificial Sequence
  source: /note=“Description of Artificial Sequence Synthetic oligonucleotide”
actggtaacccagacatgatcggt

SEQ ID NO: 59 - DNA, 29 bp, Artificial Sequence
  source: /note=“Description of Artificial Sequence Synthetic oligonucleotide”
  source: /note=“Description of Combined DNA/RNA Molecule Synthetic oligonucleotide”
accgatcatgtcuuuuutgggttaccagt

SEQ ID NO: 60 - DNA, 30 bp, Artificial Sequence
  source: /note=“Description of Artificial Sequence Synthetic oligonucleotide”
  source: /note=“Description of Combined DNA/RNA Molecule Synthetic oligonucleotide”
accgatcatgtctaucgctgggttaccagt

SEQ ID NO: 61 - DNA, 30 bp, Artificial Sequence
  source: /note=“Description of Artificial Sequence Synthetic oligonucleotide”
  source: /note=“Description of Combined DNA/RNA Molecule Synthetic oligonucleotide”
accgatcatgtctaaugctgggttaccagt

SEQ ID NO: 62 - DNA, 30 bp, Artificial Sequence
  source: /note=“Description of Artificial Sequence Synthetic oligonucleotide”
  source: /note=“Description of Combined DNA/RNA Molecule Synthetic oligonucleotide”
accgatcatgtctagagctgggttaccagt

SEQ ID NO: 63 - DNA, 30 bp, Artificial Sequence
  source: /note=“Description of Artificial Sequence Synthetic oligonucleotide”
  source: /note=“Description of Combined DNA/RNA Molecule Synthetic oligonucleotide”
accgatcatgtctaacgctgggttaccagt

SEQ ID NO: 64 - DNA, 15 bp, Artificial Sequence
  source: /note=“Description of Artificial Sequence Synthetic primer”
gacagaactagacac

SEQ ID NO: 65 - DNA, 61 bp, Artificial Sequence; identical to SEQ ID NO: 20
  source: /note=“Description of Artificial Sequence Synthetic oligonucleotide”

SEQ ID NO: 66 - DNA, 25 bp, Artificial Sequence
  source: /note=“Description of Artificial Sequence Synthetic oligonucleotide”
aagagagtagtacagcagccgtcaa

SEQ ID NO: 67 - DNA, 23 bp, Artificial Sequence
  source: /note=“Description of Artificial Sequence Synthetic probe”
cagtgaatgcgagtccgtctaag

SEQ ID NO: 68 - DNA, 24 bp, Artificial Sequence
  source: /note=“Description of Artificial Sequence Synthetic oligonucleotide”
actggtaacccagacatgatcggt

SEQ ID NO: 69 - DNA, 24 bp, Artificial Sequence
  source: /note=“Description of Artificial Sequence Synthetic oligonucleotide”
ctaatagctcctgtgccctcgtat

SEQ ID NO: 70 - DNA, 24 bp, Artificial Sequence
  source: /note=“Description of Artificial Sequence Synthetic oligonucleotide”
aatccgtcgactagcctgagaatt

SEQ ID NO: 71 - DNA, 24 bp, Artificial Sequence
  source: /note=“Description of Artificial Sequence Synthetic oligonucleotide”
cgtaccattgaatctggagacctt